(19) United States
(12) Patent Application Publication
(10) Pub. No.: US 2024/0101634 A1
(43) Pub. Date: Mar. 28, 2024

(54) hAM15-52 ANALOGUES WITH IMPROVED AMYLIN RECEPTOR (hAMY3R) POTENCY

(71) Applicant: GUBRA A/S, Hørsholm (DK)

(72) Inventors: Jens Christian Frøslev NIELSEN, Hørsholm (DK); Kristoffer Tobias, Gustav RIGBOLT, Hørsholm (DK); Esben Matzen BECH, Hørsholm (DK); Morten LUNDH, Hørsholm (DK); Paola MAGOTTI, Hørsholm (DK); Borja BALLARiN-GONZÁLEZ, Hørsholm (DK); Søren Ljungberg PEDERSEN, Borup (DK); Nieis VRANG, Hørsholm (DK)

(73) Assignee: GUBRA A/S, Hørsholm (DK)

(21) Appl. No.: 18/024,879

(22) PCT Filed: Sep. 23, 2021
(86) PCT No.: PCT/EP2021/076250

§ 371 Date: Mar. 6, 2023

(30) Foreign Application Priority Data
Sep. 24, 2020 (EP) 20198117.2

(51) Int. Cl. C07K 14/575

(52) U.S. Cl. CPC C07K 14/575

(57) ABSTRACT

The present invention relates to hAM₁₅₋₅₂ analogues with improved amylin receptor (hAMY3R) potency (hAMY3R-EC₅₀≤250 pM) and which are largely based on the sequence of the human adrenomedullin fragment hAM₁₅₋₅₂. The invention further relates to hAM₁₅₋₅₂ analogues that are selective amylin receptor (hAMY3R) agonists (hAMY3R-EC₅₀≤250 pM and an hAM1R-EC₅₀≥25 nM) and which are largely based on the sequence of the human adrenomedullin fragment hAM₁₅₋₅₂. The hAM₁₅₋₅₂ analogues according to the invention maintain the good physical stability of hAM₁₅₋₅₂. The invention further relates to pharmaceutical compositions comprising such polypeptides and their use in the treatment of a medical condition such as obesity, NASH and/or diabetes.

### FIELD OF THE INVENTION

The present invention relates to hAM₁₅₋₅₂ analogues with improved amylin receptor (hAMY3R) potency (hAMY3R-EC₅₀≤250 pM) and which are largely based on the sequence of the human adrenomedullin fragment hAM₁₅₋₅₂. The invention further relates to hAM₁₅₋₅₂ analogues that are selective amylin receptor (hAMY3R) agonists (hAMY3R-EC₅₀≤250 pM and an hAM1R-EC₅₀≥25 nM). In particularly, the present invention is based on the realization that the human adrenomedullin fragment hAM₁₅-52 may be converted into highly selective amylin receptor agonists (hAMY3R-EC₅₀≤250 pM and an hAM1R-EC₅₀≥25 nM) by replacing the amino acid in position X₁₁ in hAM₁₅₋₅₂ together with one or more of the amino acids in the positions X₄, X₃₇ and/or X₃₈ in hAM₁₅₋₅₂. Thus, replacing at least two amino acids on hAM₁₅₋₅₂ resulted in hAM₁₅₋₅₂ analogues with AMY3R and AM1R potencies comparable to hAMY₁₋₃₇ (amylin). The invention further relates to pharmaceutical compositions comprising such hAM₁₅₋₅₂ analogues and their medical use in the treatment of a medical condition, such as obesity and/or diabetes.

### BACKGROUND OF THE INVENTION

Obesity is a medical condition in which excess body fat has accumulated to the extent that it has a negative impact on health. It is affecting a huge number of individuals worldwide and increasing rapidly in certain parts of the world. The World Health Organisation (WHO) estimated that in 2016, approximately 650 million people were obese worldwide. Obesity is defined as a body mass index (BMI) above 30. Obesity is considered a major risk factor for developing a variety of medical conditions, such as cardiovascular diseases (e.g. hypertension, atherosclerosis, heart attacks or stroke), NASH musculoskeletal disorders, certain kinds of cancers, depression and diabetes type II, and hence is detrimental to human health. Cardiovascular diseases and diabetes are two main diseases associated with obesity. A large amount of research has been conducted in the obesity field in search for new treatments for obesity or obesity-related diseases and disorders.

Diabetes is a group of metabolic disorders characterized by a high blood sugar level. As of 2019, the International Diabetes Federation estimated that 463 million people are suffering from diabetes worldwide, approximately half of the individuals being diagnosed. Diabetes is divided into two types, namely type I and type II diabetes. Type I diabetes results from the pancreas's failure to produce enough insulin due to loss of beta cells caused by an autoimmune response. On the other hand, type II diabetes is a condition that begins with insulin resistance in which cells fail to respond to insulin properly and as the disease progresses may also result in a lack of insulin.

Human amylin (hAMY₁₋₃₇ or amylin) is a 37-residues peptide hormone that is co-secreted with insulin from the pancreatic β-cells with the amino acid sequence Lys-Cys-Asn-Thr-Ala-Thr-Cys-Ala-Thr-Gln-Arg-Leu-Ala-Asn-Phe-Leu-Val-His-Ser-Ser-Asn-Asn-Phe-Gly-Ala-Ile-Leu-Ser-Ser-Thr-Asn-Val-Gly-Ser-Asn-Thr-Tyr with a disulphide bridge between residues 2 and 7. Amylin supresses glucagon release and inhibits gastric emptying and hence plays an important role in maintaining glucose homeostasis by decreasing the blood sugar concentration. Therefore, amylin is a potential candidate for treating diabetes. Furthermore, amylin has been shown to reduce food intake and plays an important role in satiety, also making it a potential candidate for treating obesity. However, amylin possesses some drawbacks such as a high tendency of fibrillation, a short in vivo half-life, and chemical instability at pH 7. Thus, native amylin is suboptimal for use as a pharmaceutically active ingredient.

A large number of amylin derivatives are known in the prior art, such as the ones disclosed in WO2016/146739. These amylin analogues attempt to solve some of the known drawbacks that human amylin possesses. One successful example is the amylin analogue Pramlintide, which has been approved by the FDA for use in type I and type II diabetes. However, Pramlintide is formulated at pH 4 as it fibrillates at pH 7. Thus, there is still a need for new amylin analogues with increased chemical stability, increased metabolic stability, and/or a reduced tendency for fibrillation. In particular, amylin analogues that are stable over a broader pH range are desirable. Furthermore, there is a need for new amylin analogues with increased effectiveness through e.g. increased potency, higher efficacy, and/or longer half-lives to allow less frequent dosing and increased patient compliance. The present invention sets out to solve one or more of the problems known from the prior art by applying a new chemical strategy.

### SUMMARY OF THE INVENTION

In a first aspect, the present invention relates to hAM₁₅₋₅₂ analogues with improved amylin receptor (hAMY3R) potency (hAMY3R-EC₅₀≤250 pM). Thus, in a first aspect the invention relates to hAM₁₅₋₅₂ analogues or a pharmaceutically acceptable salt thereof comprising 38 amino acids (X₁-X₃₈), wherein the amino acid in position X₁₁ is selected from R, W or Cit and wherein the hAM₁₅₋₅₂ analogue has at least 50% homology to hAM₁₅₋₅₂ (SEQ ID NO: 1), such as at least 60% homology to hAM₁₅₋₅₂ (SEQ ID NO: 1). The inventors surprisingly found that the amino acid position X₁₁ was highly important for increasing amylin receptor potency of hAM₁₅₋₅₂. Thus, a single substitution in X₁₁ from K to R, W or Cit was enough to improve the amylin receptor potency (hAMY3R-EC₅₀≤250 pM) of hAM₁₅₋₅₂ significantly while retaining adrenomedullin potency (hAM1R).

In a second aspect, the present invention provides selective hAM₁₅₋₅₂ analogues (hAMY3R-EC₅₀≤250 pM and an hAM1R-EC₅₀≥25 nM), which act as agonists on the amylin receptor hAMY3R but which, unlike in the prior art (e.g. WO2016/146739), are derived from the backbone of the adrenomedullin fragment hAM₁₅₋₅₂. The inventors surprisingly found that each of the positions X₄, X₃₇ and X₃₈ were highly important for the hAM1R potency of hAM₁₅₋₅₂ and that hAM1R potency of hAM₁₅₋₅₂ could be abolished or reduced (hAM1R-EC₅₀≥25 nM) by certain amino acid substitutions in either of the positions X₄, X₃₇ and/or X₃₈. Thus, in a second aspect, the invention relates to hAM₁₅₋₅₂ analogues comprising 38 amino acids (X₁-X₃₈) or a pharmaceutically acceptable salt thereof, wherein the amino acid in position X₁₁ is selected from R, W or Cit and wherein the hAM₁₅₋₅₂ analogue has at least 50% homology to hAM₁₅₋₅₂ (SEQ ID NO: 1), such as at least 60% homology to hAM₁₅₋₅₂ (SEQ ID NO: 1) and further wherein X₄ is selected as F, Y, W, T, M, I, A or C; X₃₇ is selected as G, Y, S, W, T, Q, P, M, I, H, F, E, A, R, C or K; X₃₈ is selected as Hyp, Y, W, T, Q, P, M, I, H, F, E, A, R, or K, with the proviso that at least one of the positions X₄, X₃₇ or X₃₈ is not the amino acid present in hAM₁₅₋₅₂ (SEQ ID NO: 1) in said position. The inventors surprisingly found that the human adrenomedullin fragment hAM₁₅₋₅₂ may be converted into highly selective amylin receptor agonists (hAMY3R-EC₅₀ value≤250 pM and an hAM1R-EC₅₀≥25 nM) by replacing the amino acid in position X₁₁ in hAM₁₅₋₅₂ together with one or more of the amino acids in the positions X₄, X₃₇ and/or X₃₈ in hAM₁₅₋₅₂. Thus, replacing at least two amino acids on hAM₁₅₋₅₂ resulted in hAM₁₅₋₅₂ analogues with hAMY3R and hAM1R potencies comparable to amylin rather than hAM₁₅₋₅₂. One benefit of this approach is that unlike human amylin, hAM₁₅₋₅₂ is not prone to fibrillation, and by using this new chemical strategy, the inventors further envisaged that the good fibrillation properties of hAM₁₅₋₅₂ could be maintained in the new hAM₁₅₋₅₂ analogues. Therefore, the hAM₁₅₋₅₂ analogues according to the invention comprise a significant part of the backbone present in hAM₁₅₋₅₂ as an important part of the sequence (i.e. at least least 50% homology to hAM₁₅₋₅₂ (SEQ ID NO: 1)). Thus, in the second aspect of the invention, the first problem solved is the provision of new potent hAMY3R agonists with high selectivity over hAM1R, which are obtained by tweaking the polypharmacology of the hAM₁₅₋₅₂ fragment into hAMY3R agonism. The second problem solved by the second aspect is the provision of new hAMY3R agonists which are less prone to fibrillation, thereby overcoming a problem inherent to human amylin. Unlike the amylin derivatives known from the prior art, the hAM₁₅₋₅₂ analogues of the present invention possess a structurally distinct sequence belonging to the hAM₁₅₋₅₂ fragment. Thus, the hAM₁₅₋₅₂ analogues of the invention possess different physical-chemical properties compared to the amylin derivatives in the prior art, such as different solubility, chemical, physical-, and/or metabolic stability.

In a third aspect, the invention relates to hAM₁₅₋₅₂ analogues according to the first and/or second aspect for use as a medicament. More particularly, the third aspect of the invention relates to hAM₁₅₋₅₂ analogues according to the first and/or second aspect for use in treating, preventing or ameliorating a variety of diseases, disorders or conditions, such as but not limited to excess food intake, excess body weight, obesity, Binge eating disorder, Prader-Willi syndrome, dyslipidemia, metabolic diseases/disorders, diabetes I or II, impaired glucose tolerance, insulin resistance syndrome and/or NASH.

In a fourth aspect, the invention relates to pharmaceutical compositions comprising one or more of the hAM₁₅₋₅₂ analogues according to the first and/or second aspect and their medical use(s) in treating, preventing, or ameliorating a variety of diseases, disorders or conditions according to the third aspect. The pharmaceutical compositions may comprise a pharmaceutically acceptable carrier (vehicle) and/or one or more excipient(s).

In a fifth aspect, the invention relates to a method of treating a human or animal subject with one or more hAM₁₅₋₅₂ analogue(s) according to the first and/or second aspect, wherein the human or animal subject is diagnosed with or suffering from one or more of the diseases, disorders or conditions, such as but not limited to excess food intake, excess body weight, obesity, Binge eating disorder, Prader-Willi syndrome, dyslipidemia, metabolic diseases/disorders, diabetes I or II, impaired glucose tolerance, insulin resistance syndrome and/or NASH, preferably obesity, diabetes I or II and/or NASH.

The invention will now be explained in more detail from the alignment of the amylin (hAMY₁₋₃₇) and the adrenomedullin fragment (hAM₁₅₋₅₂) as shown in Table 1 below.

### Definitions

In the present context, when the disclosure refers to positions of amino acids in the hAM₁₅₋₅₂ analogues (i.e. X₁-X₃₈) that are derived/selected from hAMY₁₋₃₇ or retained/selected from the hAM₁₅₋₅₂ fragment, the following alignment shown in Table 1 applies. Thus, position X₁ in the hAM₁₅₋₅₂ analogues according to the invention corresponds to the first amino acid in human amylin (hAMY₁₋₃₇) and amino acid number 15 (hAM₁₅) in the hAM₁₅₋₅₂ fragment.

TABLE 1

| | Position X₁₋₃₈ | | | | | | | | | | | | | | | | | | |
|---|---|---|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 | 17 | 18 | 19 |
| hAM₁₅₋₅₂ | G | C | R | F | G | T | C | T | V | Q | K | L | A | H | Q | I | Y | Q | F |
| hAMY₁₋₃₇ | K | C | N | T | A | T | C | A | T | Q | R | L | A | N | F | L | V | H | S |
| | Position X₁₋₃₈ | | | | | | | | | | | | | | | | | | |
| | 20 | 21 | 22 | 23 | 24 | 25 | 26 | 27 | 28 | 29 | 30 | 31 | 32 | 33 | 34 | 35 | 36 | 37 | 38 |
| hAM₁₅₋₅₂ | T | D | K | D | K | D | N | V | A | P | R | S | K | I | S | P | Q | G | Y |
| hAMY₁₋₃₇ | S | N | N | F | - | G | A | I | L | S | S | T | N | V | G | S | N | T | Y |

According to the present invention, the hAM₁₅₋₅₂ analogues are generally amidated at the C-terminal (—CONH₂), like the native peptides; amylin and adrenomedullin. However, the hAM₁₅₋₅₂ analogues of the present invention may also have either a free carboxylic acid (—COOH) or another post-translational modification such as a methyl ester (—COOMe). In a highly preferred embodiment of the invention, the hAM₁₅₋₅₂ analogues are amidated at the C-terminal. The hAM₁₅₋₅₂ analogues according to the present invention may have a free amine (—NH₂), be N-acylated (—NHCOR), N-methylated (—NHCH₃ or —N(CH₃)₂) or deaminated at the N-terminal. The hAM₁₅₋₅₂ analogues may also be lipidated, e.g. at the N-terminal as exemplified herein, depending on the desired half-life of the polypeptides.

According to the present invention, lipidation has the usual meaning in the art. Thus, lipidation in the present context refers to the covalent attachment of a lipid optionally through a linker to hAM₁₅₋₅₂ analogues of the invention. Lipidation is typically performed to improve the pharmacokinetic profile of a polypeptide by e.g. improving metabolic stability, reducing enzymatic degradation, lowering excretion and metabolism, all in all resulting in a prolonged in vivo half-life (t_{1/2}) of the hAM₁₅₋₅₂ analogues. The hAM₁₅₋₅₂ analogues according to the invention may be lipidated or non-lipidated. The lipidated hAM₁₅₋₅₂ analogues exemplified herein are lipidated with a saturated C₂₀-diacid through various linkers as shown in table 2.

According to the present invention, the abbreviations Hyp, Cit, Aib, Aad, (NMe)G/Sar, (NMe)I have the usual meaning in the art. Thus, Hyp refers to L-hydroxyproline, Cit refers to L-citrulline, Aib refers to 2-aminoisobutyric acid, Aad refers to L-homoglutamic acid, (NMe)G refers to N-methylglycine also known as Sar or sarcosine and (NMe)I refer to N-methyl-L-isoleucine.

According to the present invention, EC₅₀ values are used as a measure of agonist potency at a given receptor. An EC₅₀ value is a measure of the concentration of a compound required to achieve half of that compound's maximal activity in a particular assay. Thus, an hAM₁₅₋₅₂ analogue according to the invention having an hAMY3R-EC₅₀ lower than the hAMY3R-EC₅₀ of hAM₁₅₋₅₂ in the given assay is considered to have higher potency or activity at the hAMY3R receptor than the adrenomedullin fragment hAM₁₅₋₅₂. Likewise, an hAM₁₅₋₅₂ analogue according to the invention having an hAM1R-EC₅₀ higher than the hAM1R-EC₅₀ of hAM₁₅₋₅₂, in the given assay may be considered to have lower potency or activity at the hAM1R receptor than the adrenomedullin fragment hAM₁₅₋₅₂.

According to the present invention, an hAM₁₅₋₅₂ analogue or derivative thereof may be in the form of a pharmaceutically acceptable salt. Thus, pharmaceutically acceptable salts are intended to include any salts that are commonly used in formulations of peptides. Such salts include both acid addition salts and basic salts, and examples may be found e.g. in Remington's Pharmaceutical Sciences, 17^{th} edition. Likewise, various solvates of the hAM₁₅₋₅₂ analogues or pharmaceutically acceptable salts thereof are also within the scope of the invention.

According to the present invention, hAM₁₅₋₅₂ analogues are short chains of amino acids that have been linked by amide bonds. In the most preferred embodiment of the invention, the hAM₁₅₋₅₂ analogues are 38 amino acids in length, not including any amino acids that function as spacers between an optional lipid and the hybrid polypeptides.

In the present context, unless otherwise stated, the amino acids are all L-amino acids (L-stereoisomer, natural amino acids). Thus, the absolute configuration of the amino acids is the (S)-configuration with the exception of L-cysteine and L-selenocysteine having the (R)-configuration.

In the present context, it should be understood that the amino acid Cys (X₂) and Cys (X₇) in the hAM₁₅₋₅₂ analogues are covalently connected by a bridge, preferably a disulfide bridge (—S—S—) such as present in hAMY₁₋₃₇ and hAM₁₅₋₅₂. The cyclization of the disulfide bridge may be performed after purification of the hybrid polypeptides or prior to cleavage from the resin in the solid-phase peptide synthesis, optionally in the presence of suitable protecting groups. The disulfide bridge may be formed spontaneously by stirring the hybrid polypeptide in the presence of oxygen or may be formed by treating the hybrid polypeptides with another suitable oxidant such as iodine (I₂), optionally in the presence of a base.

In the present context, the hAM₁₅₋₅₂ analogues or derivatives thereof according to the first or second aspect have an hAMY3R potency (EC₅₀)≤250 pM, such as ≤200 pM, preferably an hAMY3R-EC₅₀≤150 pM, such as ≤125 pM, more preferably an hAMY3R-EC₅₀≤100 pM, such as ≤75 pM, yet more preferably an hAMY3R-EC₅₀≤50 pM, such as ≤25 pM, most preferably an hAMY3R-EC₅₀≤15 pM, such as ≤10 pM. Thus, the most hAMY3R potent hAM₁₅₋₅₂ analogues may be more or less equipotent with native human amylin hAMY₁₋₃₇. For example, SEQ ID NOs: 3-44 have an hAMY3R-EC₅₀ between 6.0-10 pM and are thus more or equipotent compared to hAMY₁₋₃₇ (hAMY3R-EC₅₀=10 pM) when measured under the same assay conditions.

In the present context, the hAM₁₅₋₅₂ analogues or derivatives thereof according to the second aspect have an abolished or reduced EC₅₀ when the EC₅₀ value on hAM1R≥25 nM, such as EC₅₀ value on hAM1R≥50 nM, such as an hAM1R-EC₅₀≥100 nM, preferably an hAM1R-EC₅₀≥150 nM, such as ≥200 nM, more preferably hAM1R-EC₅₀≥250 nM, such as ≥300 nM, even more preferably an hAM1R-EC₅₀≥350 nM, such as ≥400 nM, yet more preferably an hAM1R-EC₅₀≥450 nM, such as ≥500 nM, yet more preferably an hAM1R-EC₅₀≥600 nM, such as ≥700 nM, yet more preferably an hAM1R-EC₅₀≥800 nM, such as ≥900 nM, yet more preferably an hAM1R-EC₅₀≥1000 nM, most preferably an hAM1R-EC₅₀≥5000 nM. Thus, in the present context, selectively in terms of potency towards the amylin receptor hAMY3R over the adrenomedullin receptor hAM1R (i.e. a selective amylin receptor agonist) should be understood as an hAM₁₅₋₅₂ analogue or a derivative thereof according to the second aspect having an hAMY3R-EC₅₀ value≤250 pM and an hAM1R-EC₅₀≥25 nM. Thus, a selective hAM₁₅₋₅₂ analogue or a derivative thereof has a selectivity ratio (hAM1R EC₅₀/hAMY3R EC₅₀) of at least 100. Preferably, the hAM₁₅₋₅₂ analogues or derivatives thereof have an hAMY3R-EC₅₀ value≤250 pM and an hAM1R-EC₅₀≥50 nM and thus a selectivity ratio (hAM1R EC₅₀/hAMY3R EC₅₀)≥200. More preferably, the hAM₁₅₋₅₂ analogues or derivatives thereof have an hAMY3R-EC₅₀≤200 pM, an hAM1R-EC₅₀≥100 nM and thus a selectivity ratio (hAM1R EC₅₀/hAMY3R EC₅₀)≥500. More preferably, an hAM₁₅₋₅₂ analogue or a derivative thereof has an hAMY3R-EC₅₀≤125 pM, an hAM1R-EC₅₀≥200 nM and thus a selectivity ratio (hAM1R EC₅₀/hAMY3R EC₅₀)≥1600. Even more preferably, the hAM₁₅₋₅₂ analogue or a derivative thereof has an hAMY3R-EC₅₀≤100 pM, an hAM1R-EC₅₀≥500 nM and thus a selectivity ratio (hAM1R EC₅₀/hAMY3R EC₅₀)≥5000. Most preferably, the hAM₁₅₋₅₂ analogue or a derivative thereof has an hAMY3R-EC₅₀≤50 pM, an hAM1R-EC₅₀≥1000 nM and thus a selectivity ratio (hAM1R EC₅₀/hAMY3R EC₅₀)≥20000. For example, any of SEQ ID NOs: 3-42 have a selectivity ratio of at least 6000 and most of them much higher.

A person skilled in the art is well aware that EC₅₀ values depend on the assay type and assay conditions. Thus, in the present context when the disclosure refers to an EC₅₀ value, it should be understood as an EC₅₀ value obtained when measured according to the procedure described in the examples under general protocols for cAMP assays for measuring in vitro receptor activation. However, variation may also be present within the same assay under apparently identical assay conditions due to variation in e.g. receptor expression in the cells (i.e. receptor density). Thus, in order to compare EC₅₀ values, hAMY₁₋₃₇ (SEQ ID NO: 2) and hAM₁₅₋₅₂ (SEQ ID NO: 1) have been tested as internal standards for comparison between different assay runs or even different assays. The hAMY3R-EC₅₀ and the hAM1R-EC₅₀ of hAMY₁₋₃₇ (SEQ ID NO: 2) was determined to 10 pM and 5000 nM, respectively, using the same assay conditions and cell line as the hAM₁₅₋₅₂ analogues (SEQ ID NO: 3-392). Likewise, the hAMY3R-EC₅₀ and the hAM1R-EC₅₀ of the hAM₁₅₋₅₂ (SEQ ID NO: 1) was determined to 1.3 nM and 1.1 nM, respectively, using the same assay conditions and cell line as the hAM₁₅₋₅₂ analogues (SEQ ID NO: 3-392). Thus, it follows that the hAM₁₅₋₅₂ analogues or derivatives thereof according to the first or second aspect have a relative hAMY3R-EC₅₀ ratio (hAMY3R-EC₅₀)/(hAMY₁₋₃₇-hAMY3R-EC₅₀)≤(250 pM)/(10 pM)≤25. Preferably, the relative hAMY3R-EC₅₀ ratio (hAMY3R-EC₅₀)/(hAMY₁₋₃₇-hAMY3R-EC₅₀)≤20. More preferably, the relative hAMY3R-EC₅₀ ratio (hAMY3R-EC₅₀)/(hAM₁₋₃₇-hAMY3R-EC₅₀)≤12.5. Even more preferably, the relative hAMY3R-EC₅₀ ratio (hAMY3R-EC₅₀)/(hAMY₁₋₃₇-hAMY3R-EC₅₀)≤10. Even more preferably, the relative hAMY3R-EC₅₀ ratio (hAMY3R-EC₅₀)/(hAMY₁₋₃₇-hAMY3R-EC₅₀)≤5. Yet more preferably, the relative hAMY3R-EC₅₀ ratio (hAMY3R-EC₅₀)/(hAMY₁₋₃₇-hAMY3R-EC₅₀)≤2. Most preferably, the relative hAMY3R-EC₅₀ ratio (hAMY3R-EC₅₀)/(hAMY₁₋₃₇-AMY3R-EC₅₀)≤1 such that the hAM₁₅₋₅₂ analogues or derivatives thereof an equipotent or more potent than hAMY₁₋₃₇. Likewise, the selective hAM₁₅₋₅₂ analogues or derivatives thereof according to the second aspect have a relative hAMY3R-EC₅₀ ratio (hAMY3R-EC₅₀)/(hAM₁₅₋₅₂-AMY3R-EC₅₀)≤(250 pM)/(1300 pM)≤0.19 and a relative hAM1R-EC₅₀ ratio (hAM1R-EC₅₀)/(hAM₁₅₋₅₂-hAM1R-EC₅₀)≥(25 nM)/(1.1 nM)≥22.7. Preferably, the relative hAMY3R-EC₅₀ ratio (hAMY3R-EC₅₀)/(hAM₁₅₋₅₂-hAMY3R-EC₅₀)≤(250 pM)/(1300 pM)≤0.19 and a relative hAM1R-EC₅₀ ratio (hAM1R-EC₅₀)/(hAM₁₅₋₅₂-hAM1R-EC₅₀)≥(50 nM)/(1.1 nM)≥45.5. More preferably, the relative hAMY3R-EC₅₀ ratio (hAMY3R-EC₅₀)/(hAM₁₅₋₅₂-hAMY3R-EC₅₀)≤0.15 and a relative hAM1R-EC₅₀ ratio (hAM1R-EC₅₀)/(hAM₁₅₋₅₂-hAM1R-EC₅₀)≥76.9. Yet more preferably, the relative hAMY3R-EC₅₀ ratio (hAMY3R-EC₅₀)/(hAM₁₅₋₅₂-hAMY3R-EC₅₀)≤0.096 and a relative hAM1R-EC₅₀ ratio (hAM1R-EC₅₀)/(hAM₁₅₋₅₂-hAM1R-EC₅₀)≥154. Even more preferably, the relative hAMY3R-EC₅₀ ratio (hAMY3R-EC₅₀)/(hAM₁₅₋₅₂-hAMY3R-EC₅₀)≤0.077 and a relative hAM1R-EC₅₀ ratio (hAM1R-EC₅₀)/(hAM₁₅₋₅₂-hAM1R-EC₅₀)≥384. Yet more preferably, the relative hAMY3R-EC₅₀ ratio (hAMY3R-EC₅₀)/(hAM₁₅₋₅₂-AMY3R-EC₅₀)≤0.038 and a relative hAM1R-EC₅₀ ratio (hAM1R-EC₅₀)/(hAM₁₅₋₅₂-hAM1R-EC₅₀)≥769. Most preferably, the relative hAM1R-EC₅₀ ratio (hAM1R-EC₅₀)/(hAM₁₅₋₅₂-hAM1R-EC₅₀)≥2000.

According to the present invention, the term “treatment” should be understood in the broadest sense as prevention, amelioration, or treatment. Thus, treatment is also intended to include prophylactic treatment.

According to the present invention, from the aspect(s)/embodiment(s) which refer to “wherein hAM₁₅₋₅₂ analogue has (e.g.) at least 50% homology to hAM₁₅₋₅₂ (SEQ ID NO: 1)” or “a derivative thereof with (e.g.) at least 50% homology to hAM₁₅₋₅₂”, it should be understood that the hAM₁₅₋₅₂ analogue or derivative thereof has at least 50% sequence identity with hAM₁₅₋₅₂ (SEQ ID NO: 1) when aligned. As an example, SEQ ID NO: 169 illustrated below differs in two amino acids (X₁₁ and X₃₈) from hAM₁₅₋₅₂ (SEQ ID NO: 1) and thus has (38-2)/38=94.7% sequence identity to hAM₁₅₋₅₂ (SEQ ID NO: 1).

| | |
|---|---|
| SEQ ID NO: 1 | G C R F G T C T V Q K L A H Q I Y Q F T D K D K D N V A P R S K I S P Q G Y |
| Derivative X: | G C R F G T C T V Q R L A H Q I Y Q F T D K D K D N V A P R S K I S P Q G Hyp |

### DETAILED DESCRIPTION OF THE INVENTION

The Calcitonin Peptide Family

The calcitonin family of peptides consists of the hormone peptides calcitonin (CT), calcitonin gene-related peptide (CGRP), islet amyloid polypeptide (IAPP, amylin or hAMY₁₋₃₇), and adrenomedullin (hAM) as well as their precursors. hAMY₁₋₃₇ is a 37-residues peptide hormone that is co-secreted with insulin from the pancreatic β-cells with the amino acid sequence Lys-Cys-Asn-Thr-Ala-Thr-Cys-Ala-Thr-Gln-Arg-Leu-Ala-Asn-Phe-Leu-Val-His-Ser-Ser-Asn-Asn-Phe-Gly-Ala-Ile-Leu-Ser-Ser-Thr-Asn-Val-Gly-Ser-Asn-Thr-Tyr. Amylin supresses glucagon release and inhibits gastric emptying and hence plays an important role in maintaining glucose homeostasis by decreasing the blood sugar concentration. Furthermore, amylin has shown to reduce food intake and plays an important role in satiety, making it a potential candidate for treating e.g. obesity and/or diabetes. hAM is a 52-residues peptide hormone expressed in all tissues with the amino acid sequence Tyr-Arg-Gln-Ser-Met-Asn-Asn-Phe-Gln-Gly-Leu-Arg-Ser-Phe-Gly-Cys-Arg-Phe-Gly-Thr-Cys-Th r-Val-Gln-Lys-Leu-Ala-His-Gin-Ile-Tyr-GI n-Phe-Th r-Asp-Lys-Asp-Lys-Asp-Asn-Val-Ala-Pro-Arg-Ser-Lys-Ile-Ser-Pro-Gln-Gly-Tyr. It is a potent vasodilator and has shown positive influence in cardiovascular diseases, such as myocardial infarction, limb ischemia and hypertension.

The biological activity of the calcitonin protein family is generally mediated via binding to the calcitonin receptor (CTR) and the calcitonin receptor like receptor (CRLR), both belonging to family 2 of the G-protein-coupled receptors (GPCR). These receptors may be co-expressed in combination with different receptor modifying proteins (RAMP1-3) generating functional receptors for the individual peptides in the calcitonin protein family. Co-expression of CTR with RAMP1 leads to formation of a receptor for amylin and CGRP (AMY1R), co-expression of CTR with RAMP2 leads to the amylin receptor 2 (AMY2R) and co-expression of CTR with RAMP3 leads to the amylin receptor (AMY3R). Co-expression of CRLR with RAMP1 leads to a formation of a receptor for CGRP (CGRP1R), co-expression of CRLR with RAMP2 leads to a formation of a receptor for adrenomedullin (AM1R) and co-expression of CRLR with RAM P3 leads to a formation of a receptor for adrenomedullin and CGRP (AM2R).

Several of the native peptides in the calcitonin protein family show considerable overlap in pharmacology between receptors. For example, adrenomedullin is approximately 100 times less potent on AMY3R compared to hAMY₁₋₃₇. The adrenomedullin fragment (hAM₁₅₋₅₂) is almost equipotent on AMY3R and AM1R with an EC₅₀ of 1.3 nM on AMY3R and an EC₅₀ value of 1.1 nM on AM1R (said EC₅₀ value being measured according to the examples herein). hAMY₁₋₃₇ on the other hand has an EC₅₀ value of 10 pM on AMY3R while being inactive on AM1R.

Determination of Important Amino Acid Positions in hAM₁₅₋₅₂

The inventors initially hypothesized that since hAM₁₅₋₅₂ and hAMY₁₋₃₇ belong to the same family of hormones, the Calcitonin/CGRP/IAPP family, the AMY3R potency of hAM₁₅₋₅₂ could be improved or even converted into selective AMY3R agonism over hAM1R agonism, by substituting certain amino acids in hAM₁₅₋₅₂ with amino acids derived from hAMY₁₋₃₇, thereby overcoming some of the drawbacks that are inherent to hAMY₁₋₃₇. All 37 hAMY₁₋₃₇ amino acids were systematically substituted into the hAM₁₅₋₅₂. Furthermore, X₃₈ was selected as Hyp (Hydroxyproline), which is neither present in hAMY₁₋₃₇ nor in hAM₁₅₋₅₂. The inventors constructed random forest models (Breiman, L. (2001), Random Forests, Machine Learning 45(1), 5-32.) describing the relationship between peptides sequence and hAMY3R and the hAM1R potency, respectively, and systematically synthesised and screened peptide sequences. For each hAM₁₅₋₅₂ analogue, the amino acid in each sequence position were used as features, and receptor EC₅₀ values were used as response to construct the models. The “randomForest” package implemented in the statistical programming language R was used to calculate the importance of each position and the most important positions were identified to be critical for hAMY3R and/or hAM1R activity (A. Liaw and M. Wiener (2002). Classification and Regression by randomForest. R News 2(3), 18-22.). Based on this systematic analysis the inventors identified X₄, X₃₇ and X₃₈ to be important for hAM1R activity, while of less importance for hAMY3R activity. The inventors identified X₁₁ to be critical for hAMY3R activity, while of less importance for hAM1R activity. Finally, the inventors identified X₁₇ to be of some importance for hAMY3R activity, while of less importance for hAM1R activity. Thus, the inventors identified X₁₁ as a key position for improving hAMY3R potency in hAM₁₅₋₅₂ and X₄, X₃₇ and X₃₈ as key positions for eliminating hAM1R potency in hAM₁₅₋₅₂. Since X₁₇ was only of some importance for hAMY3R activity, this position was not considered a key position but only a preferred position to further improve the AMY3R potency of the hAM₁₅₋₅₂ analogues.

The systematic investigation showed that the hAMY3R potency of hAM₁₅₋₅₂ could be improved by substituting the amino acid K present in hAM₁₅₋₅₂ in position X₁₁ with the amino acid R present in hAMY₁₋₃₇. Furthermore, the systematic investigation showed that the hAM1R potency of hAM₁₅₋₅₂ could be decreased by substituting the amino acids present in position X₄, X₃₇ and X₃₈ in hAM₁₅₋₅₂ with the corresponding amino acids found in present in hAMY₁₋₃₇.

Deeper investigation of amino adds in the position X₄, X₁₁, X₁₇, X₃₇ and X₃₈.

The inventors systematically substituted representative amino acids covering representative amino acids for all the known amino acids into the 5 identified positions, at one position at the time (Table 6). As shown in example 1, all (SEQ ID: 1-360 had lost their activity on the hAM1R, with varying degree of potency on AMY3R. In order to score the individual amino acids on the 5 positions, the average EC₅₀ value for peptides containing a given amino acid in a given position was compared to the average EC₅₀ value hAM₁₅₋₅₂ analogues containing the reference amino acid in that position. Given a cut-off of 4-fold changes in potency, each amino acid in each position was scored as giving increased (I), decreased (D) or unchanged (U) potency, relative to the reference amino acid, on hAMY3R. The results are summarized in Table 6. From this data, it was shown that any of the amino acids R, W or Cit were capable of maintaining the AMY3R potency when present in position X₁₁ when compared to the reference whereas other representative amino acids decreased the potency. It was further shown that any of the amino acids Y, W, T, M, I, F, A, or C in X₄, any of the amino acids Y, W, T, Q, P, M, I, H, F, E, A, R, C or K in X₃₇ and any of the amino acids Hyp, Y, W, T, Q, P, M, I, H, F, E, A, R, or K in X₃₈ were able to maintain or improve AMY3R potency while remaining inactive on hAM1R.

Aspect 1. hAM₁₅₋₂₅ Analogues with Improved Amylin Potency

Thus, in a first aspect, the invention relates to an hAM₁₅₋₅₂ analogue or a pharmaceutically acceptable salt thereof comprising 38 amino acids (X₁-X₃₈) with an hAMY3R-EC₅₀≤250 pM, wherein the amino acid in position X₁₁ is selected from R, W or Cit and wherein the hAM₁₅₋₅₂ analogue has at least 50% homology to hAM₁₅₋₅₂, such as at least 60% homology to hAM₁₅₋₅₂, such as at least 65% homology to hAM₁₅₋₅₂, preferably at least 70% homology to hAM₁₅₋₅₂, such as at least 75% homology to hAM₁₅₋₅₂, more preferably at least 80 homology to hAM₁₅₋₅₂, such as at least 85% homology to hAM₁₅₋₅₂, (SEQ ID NO: 1). From example 1 (Table 2), it can be seen that almost all of the synthesized hAM₁₅₋₅₂ analogues have at least 50% homology to hAM₁₅₋₅₂, that a vast majority of the synthesized hAM₁₅₋₅₂ analogues have at least 60% of homology to hAM₁₅₋₅₂, and that a large portion of the synthesized hAM₁₅₋₅₂ analogues have at least 80% homology to hAM₁₅₋₅₂ (SEQ ID NO: 1). All of these hAM₁₅₋₅₂ analogues maintain the good fibrillation properties of hAM₁₅₋₅₂ (SEQ ID NO: 1) compared to hAMY₁₋₃₇ (SEQ ID NO: 2) (see example 2, Table 3). E.g., SEQ ID NO: 32 shows that the good physical stability (i.e. low fibrillation) of hAM₁₅₋₅₂ can be maintained as long as the hAM₁₅₋₅₂ analogues have at least 50% homology to hAM₁₅₋₅₂.

Thus, in a first aspect, the invention relates to a way to highly improve the hAMY3R potency of the hAM₁₅₋₅₂ fragment by a single substitution to R, W or Cit in position X₁₁, while maintaining the good physical stability (i.e. good fibrillation properties of hAM₁₅₋₅₂). These hAM₁₅₋₅₂ analogues will therefore be highly potent agonists on both hAMY3R and hAM1R (see SEQ ID NO: 361-392).

In the most preferred embodiment of the invention, X₁₁ is selected as R. In another embodiment, X₁₁ is selected as W. In yet an embodiment, X₁₁ is selected as Cit. The inventors found that this single amino acid substitution in X₁₁ provided increased hAMY3R potency to the hAM₁₅₋₅₂ analogue(s) while retaining hAM1R potency. The inventors further found that the hAMY3R potency could be maintained when X₁₇ was Y, T, Q, M, I, F, A, R or K, or even further improved by selecting X₁₇ as W or H. Thus, in an embodiment X₁₇ is selected as Y, W, T, Q, M, I, H, F, A, R or K. In a preferred embodiment, X₁₇ is selected as Y, W or H, more preferably W or H.

In an embodiment, the invention relates to an hAM₁₅₋₅₂ analogue comprising 38 amino acids (X₁-X₃₈₎ or a pharmaceutically acceptable salt thereof, wherein the amino acid in position X₁₁ is selected from R, W or Cit; X₁₇ is selected as Y, W, T, Q, M, I, H, F, A, R or K, preferably X₁₇ is selected as W or H and wherein the hAM₁₅₋₅₂ analogue has at least 50% homology to hAM₁₅₋₅₂, such as at least 60% homology to hAM₁₅₋₅₂, such as at least 65% homology to hAM₁₅₋₅₂, preferably at least 70% homology to hAM₁₅₋₅₂, such as at least 75 homology to hAM₁₅₋₅₂, more preferably at least 80% homology to hAM₁₅₋₅₂, such as at least 85% homology to hAM₁₅₋₅₂ (SEQ ID NO: 1).

In yet an embodiment, the invention relates to an hAM₁₅₋₅₂ analogue comprising 38 amino acids (X₁-X₃₈₎ or a pharmaceutically acceptable salt thereof, wherein the amino acid in position X₁₁ is R; X₁₇ is selected as Y, W, T, Q, M, I, H, F, A, R or K, preferably X₁₇ is selected as W or H and wherein the hAM₁₅₋₅₂ analogue has at least 50% homology to hAM₁₅₋₅₂, such as at least 60% homology to hAM₁₅₋₅₂, such as at least 65% homology to hAM₁₅₋₅₂, preferably at least 70% homology to hAM₁₅₋₅₂, such as at least 75% homology to hAM₁₅₋₅₂, more preferably at least 80% homology to hAM₁₅₋₅₂, such as at least 85% homology to hAM₁₅₋₅₂, (SEQ ID NO: 1).

In yet an embodiment, the invention relates to an hAM₁₅₋₅₂ analogue comprising 38 amino acids (X₁-X₃₈₎ or a pharmaceutically acceptable salt thereof, wherein the amino acid in position X₁₁ is W; X₁₇ is selected as Y, W, T, Q, M, I, H, F, A, R or K, preferably X₁₇ is selected as W or H and wherein the hAM₁₅₋₅₂ analogue has at least 50% homology to hAM₁₅₋₅₂, such as at least 60% homology to hAM₁₅₋₅₂, such as at least 65% homology to hAM₁₅₋₅₂, preferably at least 70% homology to hAM₁₅₋₅₂, such as at least 75% homology to hAM₁₅₋₅₂, more preferably at least 80% homology to hAM₁₅₋₅₂, such as at least 85% homology to hAM₁₅₋₅₂, (SEQ ID NO: 1).

In yet an embodiment, the invention relates to an hAM₁₅₋₅₂ analogue comprising 38 amino acids (X₁-X₃₈₎ or a pharmaceutically acceptable salt thereof, wherein the amino acid in position X₁₁ is Cit; X₁₇ is selected as Y, W, T, Q, M, I, H, F, A, R or K, preferably X₁₇ is selected as W or H and wherein the hAM₁₅₋₅₂ analogue has at least 50% homology to hAM₁₅₋₅₂, such as at least 60% homology to hAM₁₅₋₅₂, such as at least 65% homology to hAM₁₅₋₅₂, preferably at least 70% homology to hAM₁₅₋₅₂, such as at least 75% homology to hAM₁₅₋₅₂, more preferably at least 80% homology to hAM₁₅₋₅₂, such as at least 85% homology to hAM₁₅₋₅₂, (SEQ ID NO: 1).

In a preferred embodiment, the invention relates to an hAM₁₅₋₅₂ analogue or a pharmaceutically acceptable salt thereof comprising the amino acid sequence of formula (I):

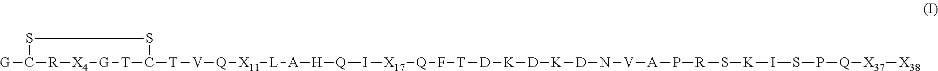

wherein
X₁₁ is selected from R, W or Cit;
X₁₇ is selected as Y, W, T, Q, M, I, H, F, A, R, or K;
X₄ is selected from F, Y, W, T, M, I, A, or C;
X₃₇ is selected from G, Y, S, W, T, Q, P, M, I, H, F, E, A, R, C, or K;
X₃₈ is selected from Hyp, Y, W, T, Q, P, M, I, H, F, E, A, R, or K;
or a derivative thereof with at least 50% homology to hAM₁₅₋₅₂, preferably at least 60% homology to hAM₁₅₋₅₂, such as at least 70% homology to hAM₁₅₋₅₂, more preferably at least 80% homology to hAM₁₅₋₅₂, even more preferably at least 85% homology to hAM₁₅₋₅₂, such as at least 90% homology to hAM₁₅₋₅₂, most preferably at least 95% homology to hAM₁₅₋₅₂, wherein X₁₁ is selected from R, W, or Cit; X₁₇ is selected as Y, W, T, Q, M, I, H, F, A, R, or K; X₄ is selected from F, Y, W, T, M, I, A or C; X₃₇ is selected from G, Y, S, W, T, Q, P, M, I, H, F, E, A, R, C, or K; X₃₈ is selected from Hyp, Y, W, T, Q, P, M, I, H, F, E, A, R, or K.

In another preferred embodiment, the invention relates to an hAM₁₅₋₅₂ analogue or a pharmaceutically acceptable salt thereof comprising the amino acid sequence of formula (I):

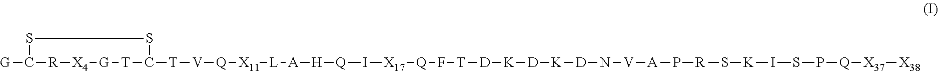

wherein
X₁₁ is selected from R;
X₁₇ is selected as Y, W, T, Q, M, I, H, F, A, R, or K;
X₄ is selected from F, Y, W, T, M, I, A, or C;
X₃₇ is selected from G, Y, S, W, T, Q, P, M, I, H, F, E, A, R, C, or K;
X₃₈ is selected from Hyp, Y, W, T, Q, P, M, I, H, F, E, A, R, or K;
or a derivative thereof with at least 50% homology to hAM₁₅₋₅₂, preferably at least 60% homology to hAM₁₅₋₅₂, such as at least 70% homology to hAM₁₅₋₅₂, more preferably at least 80% homology to hAM₁₅₋₅₂, even more preferably at least 85% homology to hAM₁₅₋₅₂, such as at least 90% homology to hAM₁₅₋₅₂, most preferably at least 95% homology to hAM₁₅₋₅₂, wherein X₁₁ is selected from R;
X₁₇ is selected as Y, W, T, Q, M, I, H, F, A, R, or K; X₄ is selected from F, Y, W, T, M, I, A, or C; X₃₇ is selected from G, Y, S, W, T, Q, P, M, I, H, F, E, A, R, C, or K; X₃₈ is selected from Hyp, Y, W, T, Q, P, M, I, H, F, E, A, R, or K.

In a more preferred embodiment, the invention relates to an hAM₁₅₋₅₂ analogue or a pharmaceutically acceptable salt thereof comprising the amino acid sequence of formula (I):

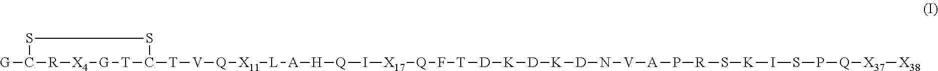

wherein
X₁₁ is selected from R;
X₁₇ is selected as Y, W, or H;
X₄ is selected from F, Y, W, T, M, I, A, or C;
X₃₇ is selected from G, Y, S, W, T, Q, P, M, I, H, F, E, A, R, C, or K;
X₃₈ is selected from Hyp, Y, W, T, Q, P, M, I, H, F, E, A, R, or K;
or a derivative thereof with at least 50% homology to hAM₁₅₋₅₂, preferably at least 60% homology to hAM₁₅₋₅₂, such as at least 70% homology to hAM₁₅₋₅₂, more preferably at least 80% homology to hAM₁₅₋₅₂, even more preferably at least 85% homology to hAM₁₅₋₅₂, such as at least 90% homology to hAM₁₅₋₅₂, most preferably at least 95% homology to hAM₁₅₋₅₂, wherein X₁₁ is selected from R; X₁₇ is selected as Y, W, or H; X₄ is selected from F, Y, W, T, M, I, A, or C; X₃₇ is selected from G, Y, S, W, T, Q, P, M, I, H, F, E, A, R, C, or K; X₃₈ is selected from Hyp, Y, W, T, Q, P, M, I, H, F, E, A, R, or K.

In yet a more preferred embodiment, the invention relates to an hAM₁₅₋₅₂ analogue or a pharmaceutically acceptable salt thereof comprising the amino acid sequence of formula (I):

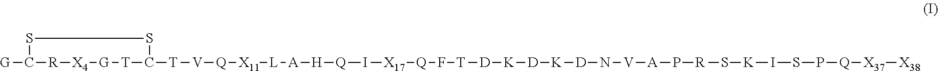

wherein
X₁₁ is selected from R;
X₁₇ is selected as Y, W, or H;
X₄ is selected from F, W, M, I, or C;
X₃₇ is selected from G, Y, S, W, T, Q, P, M, I, H, F, E, A, R, C, or K;
X₃₈ is selected from Hyp, Y, W, T, Q, P, M, I, H, F, E, A, R, or K;
or a derivative thereof with at least 50% homology to hAM₁₅₋₅₂, preferably at least 60% homology to hAM₁₅₋₅₂, such as at least 70% homology to hAM₁₅₋₅₂, more preferably at least 80% homology to hAM₁₅₋₅₂, even more preferably at least 85% homology to hAM₁₅₋₅₂, such as at least 90% homology to hAM₁₅₋₅₂, most preferably at least 95% homology to hAM₁₅₋₅₂, wherein X₁₁ is selected from R; X₁₇ is selected as Y, W, or H; X₄ is selected from F, W, M, I, or C; X₃₇ is selected from G, Y, S, W, T, Q, P, M, I, H, F, E, A, R, C, or K; X₃₈ is selected from Hyp, Y, W, T, Q, P, M, I, H, F, E, A, R, or K.

In yet a more preferred embodiment, the invention relates to an hAM₁₅₋₅₂ analogue or a pharmaceutically acceptable salt thereof comprising the amino acid sequence of formula (I):

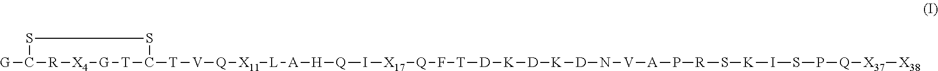

wherein
X₁₁ is selected from R;
X₁₇ is selected as Y, W, or H;
X₄ is selected from F, Y, W, T, M, I, A, or C;
X₃₇ is selected from G, Y, W, P, H, or F;
X₃₈ is selected from Hyp, Y, W, T, Q, P, M, I, H, F, E, A, R, or K;
or a derivative thereof with at least 50% homology to hAM₁₅₋₅₂, preferably at least 60% homology to hAM₁₅₋₅₂, such as at least 70% homology to hAM₁₅₋₅₂, more preferably at least 80% homology to hAM₁₅₋₅₂, even more preferably at least 85% homology to hAM₁₅₋₅₂, such as at least 90% homology to hAM₁₅₋₅₂, most preferably at least 95% homology to hAM₁₅₋₅₂, X₁₁ is selected from R; X₁₇ is selected as Y, W or H; X₄ is selected from F, Y, W, T, M, I, A, or C; X₃₇ is selected from G, Y, W, P, H, or F; X₃₈ is selected from Hyp, Y, W, T, Q, P, M, I, H, F, E, A, R, or K.

In yet a more preferred embodiment, the invention relates to an hAM₁₅₋₅₂ analogue or a pharmaceutically acceptable salt thereof comprising the amino acid sequence of formula (I):

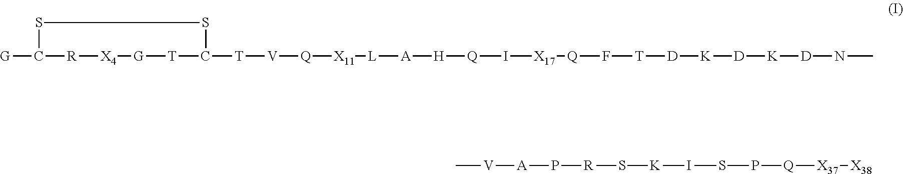

wherein
X₁₁ is selected from R;
X₁₇ is selected as Y, W, or H;
X₄ is selected from F, Y, W, T, M, I, A, or C;
X₃₇ is selected from G, Y, S, W, T, Q, P, M, I, H, F, E, A, R, C, or K;
X₃₈ is selected from Hyp, Y, W, M, or F;
or a derivative thereof with at least 50% homology to hAM₁₅₋₅₂, preferably at least 60% homology to hAM₁₅₋₅₂, such as at least 70% homology to hAM₁₅₋₅₂, more preferably at least 80% homology to hAM₁₅₋₅₂, even more preferably at least 85% homology to hAM₁₅₋₅₂, such as at least 90% homology to hAM₁₅₋₅₂, most preferably at least 95% homology to hAM₁₅₋₅₂, wherein X₁₁ is selected from R; X₁₇ is selected as Y, W, or H; X₄ is selected from F, Y, W, T, M, I, A, or C; X₃₇ is selected from G, Y, S, W, T, Q, P, M, I, H, F, E, A, R, C, or K; X₃₈ is selected from Hyp, Y, W, M, or F.

In yet a more preferred embodiment, the invention relates to an hAM₁₅₋₅₂ analogue or a pharmaceutically acceptable salt thereof comprising the amino acid sequence of formula (I):

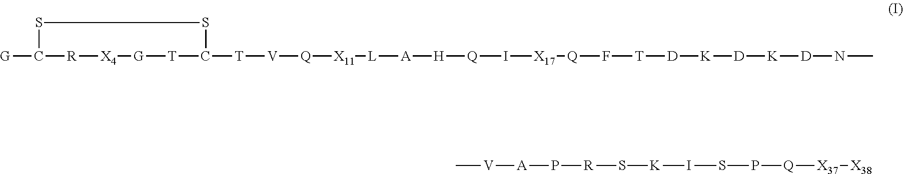

wherein
X₁₁ is selected from R;
X₁₇ is selected as Y, W, or H;
X₄ is selected from F, W, M, I, or C;
X₃₇ is selected from G, Y, W, P, H, or F;
X₃₈ is selected from Hyp, Y, W, T, Q, P, M, I, H, F, E, A, R, or K;
or a derivative thereof with at least 50% homology to hAM₁₅₋₅₂, preferably at least 60% homology to hAM₁₅₋₅₂, such as at least 70% homology to hAM₁₅₋₅₂, more preferably at least 80% homology to hAM₁₅₋₅₂, even more preferably at least 85% homology to hAM₁₅₋₅₂, such as at least 90% homology to hAM₁₅₋₅₂, most preferably at least 95% homology to hAM₁₅₋₅₂, wherein X₁₁ is selected from R; X₁₇ is selected as Y, W or H; X₄ is selected from F, W, M, I, or C; X₃₇ is selected from G, Y, W, P, H, or F; X₃₈ is selected from Hyp, Y, W, T, Q, P, M, I, H, F, E, A, R, or K.

In yet a more preferred embodiment, the invention relates to an hAM₁₅₋₅₂ analogue or a pharmaceutically acceptable salt thereof comprising the amino acid sequence of formula (I):

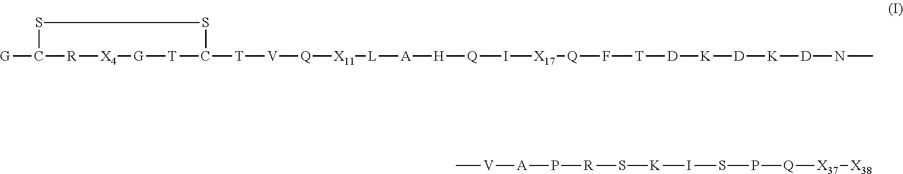

wherein
X₁₁ is selected from R;
X₁₇ is selected as Y, W, or H;
X₄ is selected from F, W, M, I, or C;
X₃₇ is selected from G, Y, S, W, T, Q, P, M, I, H, F, E, A, R, C, or K;
X₃₈ is selected from Hyp, Y, W, M, or F;
or a derivative thereof with at least 50% homology to hAM₁₅₋₅₂, preferably at least 60% homology to hAM₁₅₋₅₂, such as at least 70% homology to hAM₁₅₋₅₂, more preferably at least 80% homology to hAM₁₅₋₅₂, even more preferably at least 85% homology to hAM₁₅₋₅₂, such as at least 90% homology to hAM₁₅₋₅₂, most preferably at least 95% homology to hAM₁₅₋₅₂, wherein X₁₁ is selected from R; X₁₇ is selected as Y, W, or H; X₄ is selected from F, W, M, I, or C; X₃₇ is selected from G, Y, S, W, T, Q, P, M, I, H, F, E, A, R, C, or K; X₃₈ is selected from Hyp, Y, W, M, or F.

In yet a more preferred embodiment, the invention relates to an hAM₁₅₋₅₂ analogue or a pharmaceutically acceptable salt thereof comprising the amino acid sequence of formula (I):

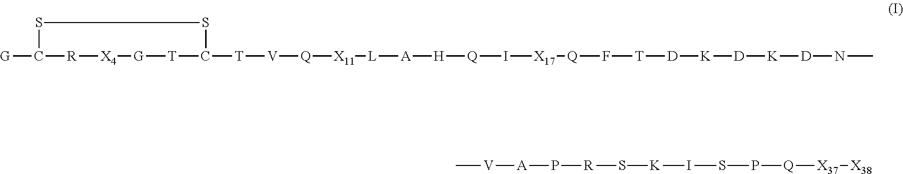

wherein
X₁₁ is selected from R;
X₁₇ is selected as Y, W, or H;
X₄ is selected from F, Y, W, T, M, I, A, or C;
X₃₇ is selected from G, Y, W, P, H, or F;
X₃₈ is selected from Hyp, Y, W, M, or F;
or a derivative thereof with at least 50% homology to hAM₁₅₋₅₂, preferably at least 60% homology to hAM₁₅₋₅₂, such as at least 70% homology to hAM₁₅₋₅₂, more preferably at least 80% homology to hAM₁₅₋₅₂, even more preferably at least 85% homology to hAM₁₅₋₅₂, such as at least 90% homology to hAM₁₅₋₅₂, most preferably at least 95% homology to hAM₁₅₋₅₂, wherein X₁₁ is selected from R; X₁₇ is selected as Y, W, or H; X₄ is selected from F, Y, W, T, M, I, A, or C; X₃₇ is selected from G, Y, W, P, H, or F; X₃₈ is selected from Hyp, Y, W, M, or F.

In yet an even more preferred embodiment, the invention relates to an hAM₁₅₋₅₂ analogue or a pharmaceutically acceptable salt thereof comprising the amino acid sequence of formula (I):

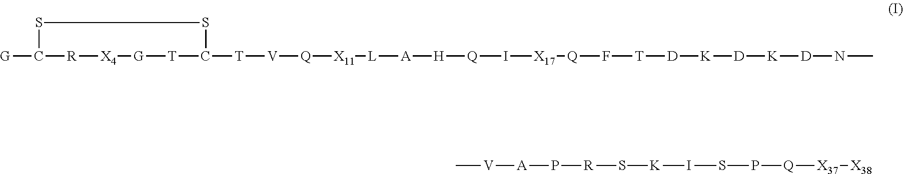

wherein
X₁₁ is selected from R;
X₁₇ is selected as Y, W, or H;
X₄ is selected from F, W, M, I, or C;
X₃₇ is selected from G, Y, W, P, H, or F;
X₃₈ is selected from Hyp, Y, W, M, or F;
or a derivative thereof with at least 50% homology to hAM₁₅₋₅₂, preferably at least 60% homology to hAM₁₅₋₅₂, such as at least 70% homology to hAM₁₅₋₅₂, more preferably at least 80% homology to hAM₁₅₋₅₂, even more preferably at least 85% homology to hAM₁₅₋₅₂, such as at least 90% homology to hAM₁₅₋₅₂, most preferably at least 95% homology to hAM₁₅₋₅₂, wherein X₁₁ is selected from R; X₁₇ is selected as Y, W or H; X₄ is selected from F, W, M, I, or C; X₃₇ is selected from G, Y, W, P, H, or F; X₃₈ is selected from Hyp, Y, W, M, or F.

In yet a more preferred embodiment, the invention relates to an hAM₁₅₋₅₂ analogue comprising the amino acid sequence of formula (I):

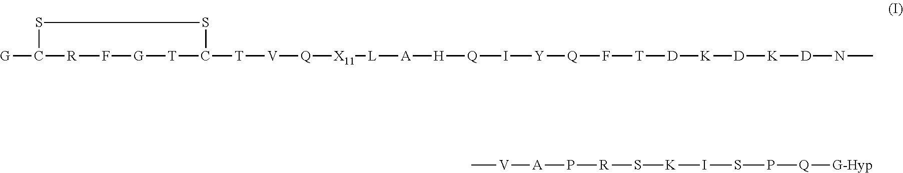

wherein, X₁₁ is selected from R, W, or Cit

or a derivative thereof with at least 50% homology to hAM₁₅₋₅₂, least 60% homology to hAM₁₅₋₅₂, such as at least 65% homology to hAM₁₅₋₅₂, preferably at least 70% homology to hAM₁₅₋₅₂, such as at least 75 homology to hAM₁₅₋₅₂, more preferably at least 80% homology to hAM₁₅₋₅₂, such as at least 85% homology to hAM₁₅₋₅₂, most preferably at least 90% homology to hAM₁₅₋₅₂ to hAM₁₅₋₅₂, such as at least 95% homology to hAM₁₅₋₅₂, wherein, X₁₁ is selected from R, W, or Cit.

In a most preferred embodiment, the invention relates to an hAM₁₅₋₅₂ analogue of SEQ ID NO: 3-392 or a derivative thereof with at least 90% homology to any one of SEQ ID NO: 3-392, preferably at least 95 homology any one of SEQ ID NO: 3-392, more preferably at least 96% homology any one of SEQ ID NO: 3-392 most preferably at least 97% homology any one of SEQ ID NO: 3-392 with the proviso that a derivative thereof has R, W, or Cit in position X₁₁, preferably R in position X₁₁.

In yet a most preferred embodiment, the invention relates to an hAM₁₅₋₅₂ analogue of SEQ ID NO: 3-392 or a derivative thereof with at least 90% homology to any one of SEQ ID NO: 3-392, preferably at least 95 homology to any one of SEQ ID NO: 3-392, more preferably at least 96% homology any one of SEQ ID NO: 3-392, most preferably at least 97% homology any one of SEQ ID NO: 3-278, with the proviso that a derivative thereof has the same amino acid in position X₁₁, as the respective SEQ ID NO: 3-392 from it is derived.

Aspect 2: Selective hAM₁₅₋₅₂ Analogues with Improved Amylin Potency

Number of Positions Needed to Abolish or Reduce AM1R Potency in hAM₁₅₋₅₂.

The inventors found that hAM1R potency could be abolished or reduced in hAM₁₅₋₅₂ by changing at least one amino acid in any of the positions X₄, X₃₇ or X₃₈ since all of them were critical for hAM1R potency.

Decreasing hAM1R Potency with Amino Adds in Position X₄, X₃₇ or X₃₈.

X₄: The inventors found that the amino acids Y, W, T, M, I, A or C in position X₄ were capable of decreasing the hAM1R potency. Thus, in an embodiment, X₄ is selected from Y, W, T, M, I, A or C. Furthermore, the amino acids W, M, I or C in position X₄ were most effective at improving hAMY3R potency (see example 5, table 6). Thus, in a highly preferred embodiment of the invention, X₄ is selected as W, M, I or C. Since the inventors found that a single position chosen among X₄, X₃₇ or X₃₈ was enough to abolish or reduce AM1R potency it follows that in some embodiments X₄ is F (i.e. the native amino acid present in hAM₁₅₋₅₂ (SEQ ID NO: 1) in said position) and the AM1R potency is abolished or reduced using position X₃₇ and/or X₃₈. Thus, in any of the above embodiments for X₄, F may be included and the AM1R potency abolished or reduced using position X₇ and/or X₃₈.

X₃₇; Likewise, the inventors found that the amino acids Y, S, W, T, Q, P, M, I, H, F, E, A, R, C in position X₃₇ were capable of decreasing the hAM1R potency. Thus, in an embodiment, X₃₇ is selected from Y, S, W, T, Q, P, M, I, H, F, E, A, R, C. Furthermore, the amino acids Y, W, P, H or F in position X₃₇ were most effective at improving hAMY3R potency (see example 5, table 6). Thus, in a highly preferred embodiment of the invention, X₃₇ is selected as Y, W, P, H or F. Since the inventors found that a single position chosen among X₄, X₃₇ or X₃₈ was enough to abolish or reduce AM1R potency it follows that in some embodiments X₃₇ is G (i.e. the native amino present in hAM₁₅₋₅₂ (SEQ ID NO: 1) in said position), and the AM1R potency is abolished or reduced using position X₄ and/or X₃₈. Thus, in any of the above embodiments for X₃₇, G may be included and the AM1R potency abolished or reduced using position X₄ and/or X₃₈.

X₃₈: Likewise, the inventors found that the amino acids Hyp, W, T, Q, P, M, I, H, F, E, A, R, or K in position X₃₈ were capable of decreasing the hAM1R potency. Thus, in an embodiment, X₃₈ is selected from Hyp, W, T, Q, P, M, I, H, F, E, A, R, or K. Furthermore, the amino acids Hyp, W, M or F were most effective at improving hAMY3R potency (see example 5, table 6). Thus, in a highly preferred embodiment of the invention, X₃₈ is selected as Hyp, W, M or F. In the most preferred embodiment of the invention, X₃₈ is selected as Hyp. Since the inventors found that a single position chosen among X₄, X₃₇ or X₃₈ was enough to abolish or reduce AM1R potency it follows that in some embodiments X₃₈ is Y (i.e. the native amino present in hAM₁₅₋₅₂ (SEQ ID NO: 1) in said position), and the AM1R potency is abolished or reduced using position X₄ and/or X₃₇. Thus, in any of the above embodiments for X₃₈, Y may be included and the AM1R potency abolished or reduced using position X₄ and/or X₃₇.

A simple illustration of aspect 2 of the invention can be seen by comparing the alignment of hAM₁₅₋₅₂ (SEQ ID NO: 1) and SEQ ID NO: 169 shown below.

SEQ ID NO: 1
G C R F G T C T V Q K L A H Q I Y Q F T D K D K D N
V A P R S K I S P Q G Y
SEQ ID NO: 169
G C R F G T C T V Q R L A H Q I Y Q F T D K D K D N
V A P R S K I S P Q G Hyp

SEQ ID NO:1 hAMY3R EC₅₀ 1.274 nM and hAM1R EC₅₀ 1.113 nM
SEQ ID NO:169 hAMY3R EC₅₀ 0.063 nM and hAM1R EC₅₀ 2981.0 nM

As can be seen, the R in position X₁₁ has improved the potency on AMY3R approximately 20-fold, and the Hyp in position X₃₈ has decreased the potency on AM1R approximately 2500-fold. Therefore, only two substitutions have converted hAM₁₅₋₅₂ into an amylin agonist with same pharmacological profile as hAMY₁₋₃₇. At the same time, SEQ ID NO: 169 has maintained the physical stability (ThT signal: 2.700%) of hAM₁₅₋₅₂ (ThT signal: 2.600%) compared to hAMY₁₋₃₇ (ThT signal: 100.0%) (see example 2, Table 3).

Thus, in a second aspect, the invention relates to a way to more or less completely abolish hAM1R potency of the hAM₁₅₋₅₂ fragment by one or more substitutions in position X₄, X₃₇ and/or X₃₈ and at the same time improve or maintain sufficient AMY3R potency. These hAM₁₅₋₅₂ analogues will therefore be highly potent and selective hAMY3R analogues. At the same time these hAM₁₅₋₅₂ analogues maintain the physical stability of hAM₁₅₋₅₂ due to the sequence homology (identity) with hAM₁₅₋₅₂. As shown in example 2, Table 3, the good physical stability of hAM₁₅₋₅₂ may be maintained or even improved in the peptides (due to at least 50 homology to hAM₁₅₋₅₂), while providing new hAMY3R agonists that are as potent and selective as natural human amylin. In other words, the second aspect of the present invention has created a functional human amylin analogue disguised in an adrenomedullin backbone.

Increasing hAMY3R Potency with Amino Acids in Position X₁₇.

The inventors further found that the hAMY3R potency could be maintained when X₁₇ as Y, T, Q, M, I, F, A, R, or K, or even further improved when X₁₇ was W or H. Thus, in an embodiment, X₁₇ is selected as Y, W, T, Q, M, I, H, F, A, R, or K. In a preferred embodiment, X₁₇ is selected as W, or H.

Thus, in a second aspect, the invention relates to hAM₁₅₋₅₂ analogues comprising 38 amino acids (X₁-X₃₈) or a pharmaceutically acceptable salt thereof, wherein the amino acid in position X₁₁ is selected from R, W or Cit and wherein the hAM₁₅₋₅₂ analogue has at least 50% homology to hAM₁₅₋₅₂, such as at least 55% homology to hAM₁₅₋₅₂, such as at least 60% homology to hAM₁₅₋₅₂, such as at least 65% homology to hAM₁₅₋₅₂, preferably at least 70% homology to hAM₁₅₋₅₂, such as at least 75% homology to hAM₁₅₋₅₂, more preferably at least 80% homology to hAM₁₅₋₅₂, such as at least 85% homology to hAM₁₅₋₅₂, most preferably at least 90% homology to hAM₁₅₋₅₂, such as at least 95% homology to hAM₁₅₋₅₂ (SEQ ID NO: 1) and further wherein X₄ is selected as F, Y, W, T, M, I, A or C; X₃₇ is selected as G, Y, S, W, T, Q, P, M, I, H, F, E, A, R, C or K; X₃₈ is selected as Hyp, Y, W, T, Q, P, M, I, H, F, E, A, R, or K, with the proviso that at least one of the positions X₄, X₃₇ or X₃₈ is not the amino acid present in hAM₁₅₋₅₂ (SEQ ID NO: 1) in said position, more preferably, at least two of the positions X₄, X₃₇ or X₃₈ is not the amino acid present in hAM₁₅₋₅₂ (SEQ ID NO: 1) in said position. In the second aspect, the hAM₁₅₋₅₂ analogues have an hAMY3R-EC₅₀≤250 pM and an hAM1R-EC₅₀≥25 nM.

In an embodiment, X₄ is selected as F, W, M, I or C; X₃₇ is selected as G, Y, S, W, T, Q, P, M, I, H, F, E, A, R, C, or K; X₃₈ is selected as Hyp, Y, W, T, Q, P, M, I, H, F, E, A, R, or K, with the proviso that at least one of the positions X₄, X₃₇, or X₃₈ is not the amino acid present in hAM₁₅₋₅₂ (SEQ ID NO: 1) in said position, more preferably, at least two of the positions X₄, X₃₇, or X₃₈ are not the amino acid present in hAM₁₅₋₅₂ (SEQ ID NO: 1) in said position.

In an embodiment, X₄ is selected as F, Y, W, T, M, I, A or C; X₃₇ is selected as G, Y, W, P, H or F; X₃₈ is selected as Hyp, Y, W, T, Q, P, M, I, H, F, E, A, R, or K, with the proviso that at least one of the positions X₄, X₃₇ or X₃₈ is not the amino acid present in hAM₁₅₋₅₂ (SEQ ID NO: 1) in said position, more preferably, at least two of the positions X₄, X₃₇ or X₃₈ are not the amino acid present in hAM₁₅₋₅₂ (SEQ ID NO: 1) in said position.

In an embodiment, X₄ is selected as F, Y, W, T, M, I, A or C; X₃₇ is selected as G, Y, S, W, T, Q, P, M, I, H, F, E, A, R, C or K; X₃₈ is selected as Hyp, Y, W, M or F, with the proviso that at least one of the positions X₄, X₃₇ or X₃₈ is not the amino acid present in hAM₁₅₋₅₂ (SEQ ID NO: 1) in said position, more preferably, at least two of the positions X₄, X₃₇, or X₃₈ is not the amino acid present in hAM₁₅₋₅₂ (SEQ ID NO: 1) in said position.

In an embodiment, X₄ is selected as F, W, M, I, or C; X₃₇ is selected as G, Y, W, P, H, or F; X₃₈ is selected as Hyp, Y, W, M or F, with the proviso that at least one of the positions X₄, X₃₇ or X₃₈ is not the amino acid present in hAM₁₅₋₅₂ (SEQ ID NO: 1) in said position, more preferably, at least two of the positions X₄, X₃₇, or X₃₈ are not the amino acid present in hAM₁₅₋₅₂ (SEQ ID NO: 1) in said position.

In a preferred embodiment, the invention relates to an hAM₁₅₋₅₂ analogue or a pharmaceutically acceptable salt thereof comprising the amino acid sequence of formula (I):

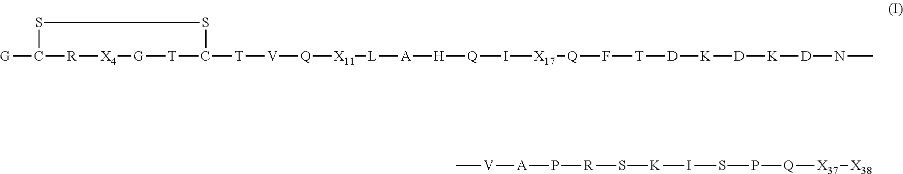

wherein
X₁₁ is selected from R, W, or Cit;
X₁₇ is selected as Y, W, T, Q, M, I, H, F, A, R, K;
X₄ is selected from F, Y, W, T, M, I, A, or C;
X₃₇ is selected from G, Y, S, W, T, Q, P, M, I, H, F, E, A, R, C, or K;
X₃₈ is selected from Hyp, Y, W, T, Q, P, M, I, H, F, E, A, R, or K;
and further wherein at least one of the positions X₄, X₃₇ or X₃₈ is not the amino acid present in hAM₁₅₋₅₂ (SEQ ID NO: 1) in said position;
or a derivative thereof with at least 50% homology to hAM₁₅₋₅₂, such as at least 55% homology to hAM₁₅₋₅₂, such as at least 60% homology to hAM₁₅₋₅₂, such as at least 65% homology to hAM₁₅₋₅₂, preferably at least 70% homology to hAM₁₅₋₅₂, such as at least 75% homology to hAM₁₅₋₅₂, more preferably at least 80 homology, even more preferably at least 85% homology, such as at least 90% homology, most preferably at least 95% homology, wherein X₁₁ is selected from R, W, or Cit; X₁₇ is selected as Y, W, T, Q, M, I, H, F, A, R, K; X₄ is selected from F, Y, W, T, M, I, A or C; X₃₇ is selected from G, Y, S, W, T, Q, P, M, I, H, F, E, A, R, C or K; X₃₈ is selected from Hyp, Y, W, T, Q, P, M, I, H, F, E, A, R, or K; and further wherein at least one of the positions X₄, X₃₇ or X₃₈ is not the amino acid present in hAM₁₅₋₅₂ (SEQ ID NO: 1) in said position.

In yet a more preferred embodiment, the invention relates to an hAM₁₅₋₅₂ analogue or a pharmaceutically acceptable salt thereof comprising the amino acid sequence of formula (I):

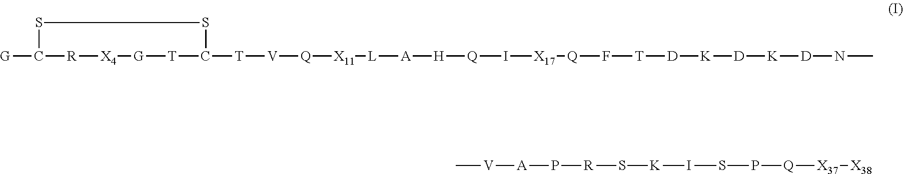

wherein
X₁₁ is selected from R, W, or Cit;
X₁₇ is selected as Y, W, T, Q, M, I, H, F, A, R or K;
X₄ is selected from F, W, M, I or C;
X₃₇ is selected from G, Y, S, W, T, Q, P, M, I, H, F, E, A, R, C or K; X₃₈ is selected from Hyp, Y, W, T, Q, P, M, I, H, F, E, A, R, or K;
and further wherein at least one of the positions X₄, X₃₇ or X₃₈ is not the amino acid present in hAM₁₅₋₅₂ (SEQ ID NO: 1) in said position;
or a derivative thereof with at least 50% homology to hAM₁₅₋₅₂, such as at least 55% homology to hAM₁₅₋₅₂, such as at least 60% homology to hAM₁₅₋₅₂, such as at least 65% homology to hAM₁₅₋₅₂, preferably at least 70% homology to hAM₁₅₋₅₂, such as at least 75% homology to hAM₁₅₋₅₂, more preferably at least 80 homology, such as at least 85% homology, more preferably at least 90% homology, most preferably at least 95% homology, wherein X₁₁ is selected from R, W or Cit; X₁₇ is selected as Y, W, T, Q, M, I, H, F, A, R or K; X₄ is selected from F, W, M, I or C; X₃₇ is selected from G, Y, S, W, T, Q, P, M, I, H, F, E, A, R, C or K; X₃₈ is selected from Hyp, Y, W, T, Q, P, M, I, H, F, E, A, R, or K; and further wherein at least one of the positions X₄, X₃₇ or X₃₈ is not the amino acid present in hAM₁₅₋₅₂ (SEQ ID NO: 1) in said position.

In another more preferred embodiment, the invention relates to an hAM₁₅₋₅₂ analogue or a pharmaceutically acceptable salt thereof comprising the amino acid sequence of formula (I):

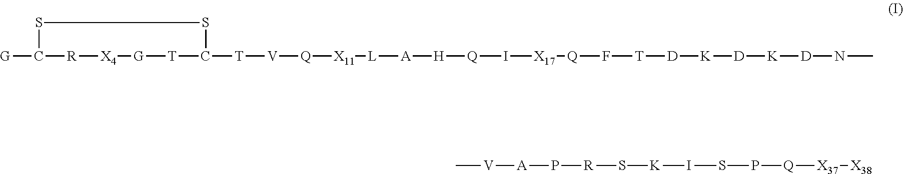

wherein amino acid present in hAM₁₅₋₅₂ (SEQ
X₁₁ is selected from R, W, or Cit; at least 55% homology to hAM₁₅₋₅₂,
X₁₇ is selected as Y, W, T, Q, M, I, H, F, A, R, or K;
X₄ is selected from F, Y, W, T, M, I, A, or C;
X₃₇ is selected from G, Y, W, P, H, or F;
X₃₈ is selected from Hyp, Y, W, T, Q, P, M, I, H, F, E, A, R, or K;
and further wherein at least one of the positions X₄, X₃₇ or X₃₈ is not the ID NO: 1) in said position;
or a derivative thereof with at least 50% homology to hAM₁₅₋₅₂, such as such as at least 60% homology to hAM₁₅₋₅₂, such as at least 65% homology to hAM₁₅₋₅₂, preferably at least 70% homology to hAM₁₅₋₅₂, such as at least 75% homology to hAM₁₅₋₅₂, more preferably at least 80 homology, such as at least 85% homology, more preferably at least 90% homology, most preferably at least 95% homology, wherein X₁₁ is selected from R, W, or Cit; X₁₇ is selected as Y, W, T, Q, M, I, H, F, A, R, or K; X₄ is selected from F, Y, W, T, M, I, A, or C; X₃₇ is selected from G, Y, W, P, H, or F; X₃₈ is selected from Hyp, Y, W, T, Q, P, M, I, H, F, E, A, R, or K; and further wherein at least one of the positions X₄, X₃₇, or X₃₈ is not the amino acid present in hAM₁₅₋₅₂ (SEQ ID NO: 1) in said position.

In yet a more preferred embodiment, the invention relates to an hAM₁₅₋₅₂ analogue or a pharmaceutically acceptable salt thereof comprising the amino acid sequence of formula (I):

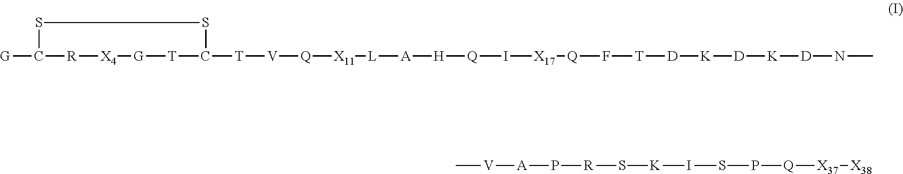

wherein
X₁₁ is selected from R, W, or Cit;
X₁₇ is selected as Y, W, T, Q, M, I, H, F, A, R, or K;
X₄ is selected from F, Y, W, T, M, I, A, or C;
X₃₇ is selected from G, Y, S, W, T, Q, P, M, I, H, F, E, A, R, C, or K;
X₃₈ is selected from Hyp, Y, W, M, or F;
and further wherein at least one of the positions X₄, X₃₇ or X₃₈ is not the amino acid present in hAM₁₅₋₅₂ (SEQ ID NO: 1) in said position;
or a derivative thereof with at least 50% homology to hAM₁₅₋₅₂, such as at least 55% homology to hAM₁₅₋₅₂, such as at least 60% homology to hAM₁₅₋₅₂, such as at least 65% homology to hAM₁₅₋₅₂, preferably at least 70% homology to hAM₁₅₋₅₂, such as at least 75% homology to hAM₁₅₋₅₂, more preferably at least 80 homology, such as at least 85% homology, more preferably at least 90% homology, most preferably at least 95% homology, wherein X₁₁ is selected from R, W, or Cit; X₁₇ is selected as Y, W, T, Q, M, I, H, F, A, R, or K; X₄ is selected from F, Y, W, T, M, I, A, or C; X₃₇ is selected from G, Y, S, W, T, Q, P, M, I, H, F, E, A, R, C, or K; X₃₈ is selected from Hyp, Y, W, M, or F; and further wherein at least one of the positions X₄, X₃₇, or X₃₈ is not the amino acid present in hAM₁₅₋₅₂ (SEQ ID NO: 1) in said position.

In yet a more preferred embodiment, the invention relates to an hAM₁₅₋₅₂ analogue or a pharmaceutically acceptable salt thereof comprising the amino acid sequence of formula (I):

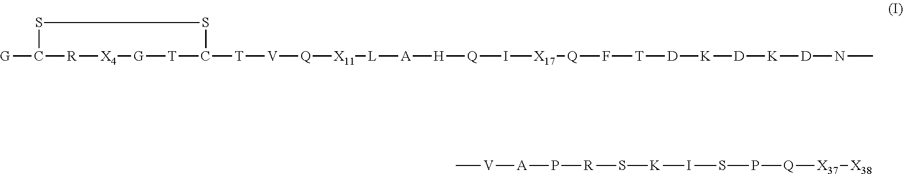

wherein
X₁₁ is selected from R, W, or Cit;
X₁₇ is selected as Y, W, T, Q, M, I, H, F, A, R, or K;
X₄ is selected from F, W, M, I, or C;
X₃₇ is selected from G, Y, W, P, H, or F;
X₃₈ is selected from Hyp, Y, W, T, Q, P, M, I, H, F, E, A, R, or K;
and further wherein at least one of the positions X₄, X₃₇ or X₃₈ is not the amino acid present in hAM₁₅₋₅₂ (SEQ ID NO: 1) in said position;
or a derivative thereof with at least 50% homology to hAM₁₅₋₅₂, such as at least 55% homology to hAM₁₅₋₅₂, such as at least 60% homology to hAM₁₅₋₅₂, such as at least 65% homology to hAM₁₅₋₅₂, preferably at least 70% homology to hAM₁₅₋₅₂, such as at least 75% homology to hAM₁₅₋₅₂, more preferably at least 80 homology to hAM₁₅₋₅₂, such as at least 85% homology to hAM₁₅₋₅₂, more preferably at least 90% homology to hAM₁₅₋₅₂, most preferably at least 95% homology to hAM₁₅₋₅₂, wherein X₁₁ is selected from R, W, or Cit; X₁₇ is selected as Y, W, T, Q, M, I, H, F, A, R, or K; X₄ is selected from F, W, M, I, or C; X₃₇ is selected from G, Y, W, P, H, or F; X₃₈ is selected from Hyp, Y, W, T, Q, P, M, I, H, F, E, A, R, or K; and further wherein at least one of the positions X₄, X₃₇ or X₃₈ is not the amino acid present in hAM₁₅₋₅₂ (SEQ ID NO: 1) in said position.

In a preferred embodiment of the invention relates to an hAM₁₅₋₅₂ analogue or a pharmaceutically acceptable salt thereof comprising the amino acid sequence of formula (I):

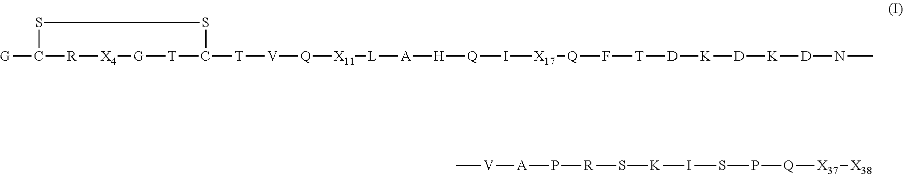

wherein
X₁₁ is selected from R, W, or Cit;
X₁₇ is selected as Y, W, T, Q, M, I, H, F, A, R, or K;
X₄ is selected from F, W, M, I, or C;
X₃₇ is selected from G, Y, S, W, T, Q, P, M, I, H, F, E, A, R, C, or K;
X₃₈ is selected from Hyp, Y, W, M, or F;
and further wherein at least one of the positions X₄, X₃₇ or X₃₈ is not the amino acid present in hAM₁₅₋₅₂ (SEQ ID NO: 1) in said position;
or a derivative thereof with at least 50% homology to hAM₁₅₋₅₂, such as at least 55% homology to hAM₁₅₋₅₂, such as at least 60% homology to hAM₁₅₋₅₂, such as at least 65% homology to hAM₁₅₋₅₂, preferably at least 70% homology to hAM₁₅₋₅₂, such as at least 75% homology to hAM₁₅₋₅₂, more preferably at least 80 homology to hAM₁₅₋₅₂, such as at least 85% homology to hAM₁₅₋₅₂, more preferably at least 90% homology to hAM₁₅₋₅₂, most preferably at least 95% homology to hAM₁₅₋₅₂, wherein X₁₁ is selected from R, W, or Cit; X₁₇ is selected as Y, W, T, Q, M, I, H, F, A, R, or K; X₄ is selected from F, W, M, I, or C; X₃₇ is selected from G, Y, S, W, T, Q, P, M, I, H, F, E, A, R, C, or K; X₃₈ is selected from Hyp, Y, W, M, or F; and further wherein at least one of the positions X₄, X₃₇ or X₃₈ is not the amino acid present in hAM₁₅₋₅₂ (SEQ ID NO: 1) in said position.

In a preferred embodiment, the invention relates to an hAM₁₅₋₅₂ analogue or a pharmaceutically acceptable salt thereof comprising the amino acid sequence of formula (I):

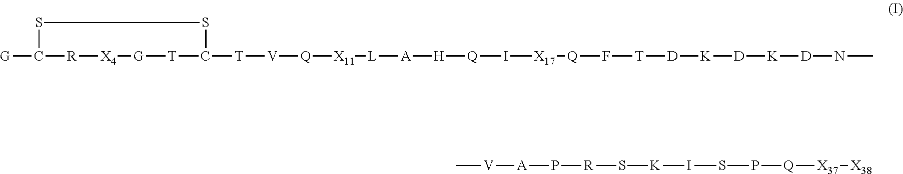

wherein
X₁₁ is selected from R, W, or Cit;
X₁₇ is selected as Y, W, T, Q, M, I, H, F, A, R, or K;
X₄ is selected from F, Y, W, T, M, I, A, or C;
X₃₇ is selected from G, Y, W, P, H, or F;
X₃₈ is selected from Hyp, Y, W, M, or F;
and further wherein at least one of the positions X₄, X₃₇ or X₃₈ is not the amino acid present in hAM₁₅₋₅₂ (SEQ ID NO: 1) in said position;
or a derivative thereof with at least 50% homology to hAM₁₅₋₅₂, such as at least 55% homology to hAM₁₅₋₅₂, such as at least 60% homology to hAM₁₅₋₅₂, such as at least 65% homology to hAM₁₅₋₅₂, preferably at least 70% homology to hAM₁₅₋₅₂, such as at least 75% homology to hAM₁₅₋₅₂, more preferably at least 80 homology, such as at least 85% homology to hAM₁₅₋₅₂, more preferably at least 90% homology to hAM₁₅₋₅₂, most preferably at least 95% homology to hAM₁₅₋₅₂, wherein X₁₁ is selected from R, W, or Cit; X₁₇ is selected as Y, W, T, Q, M, I, H, F, A, R, or K; X₄ is selected from F, Y, W, T, M, I, A or C; X₃₇ is selected from G, Y, W, P, H or F; X₃₈ is selected from Hyp, Y, W, M, or F; and further wherein at least one of the positions X₄, X₃₇, or X₃₈ is not the amino acid present in hAM₁₅₋₅₂ (SEQ ID NO: 1) in said position.

In a more preferred embodiment, the invention relates to an hAM₁₅₋₅₂ analogue or a pharmaceutically acceptable salt thereof comprising the amino acid sequence of formula (I):

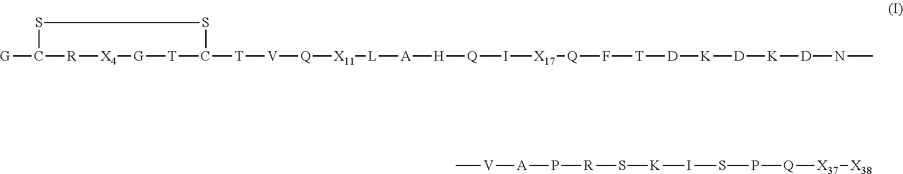

wherein
X₁₁ is selected from R, W, or Cit;
X₁₇ is selected as Y, W, T, Q, M, I, H, F, A, R, or K;
X₄ is selected from F, W, M, I, or C;
X₃₇ is selected from G, Y, W, P, H, or F;
X₃₈ is selected from Hyp, Y, W, M, or F;
and further wherein at least one of the positions X₄, X₃₇ or X₃₈ is not the amino acid present in hAM₁₅₋₅₂ (SEQ ID NO: 1) in said position;
or a derivative thereof with at least 50% homology to hAM₁₅₋₅₂, such as at least 55% homology to hAM₁₅₋₅₂, such as at least 60% homology to hAM₁₅₋₅₂, such as at least 65% homology to hAM₁₅₋₅₂, preferably at least 70% homology to hAM₁₅₋₅₂, such as at least 75% homology to hAM₁₅₋₅₂, more preferably at least 80 homology to hAM₁₅₋₅₂, such as at least 85% homology to hAM₁₅₋₅₂, more preferably at least 90% homology to hAM₁₅₋₅₂, most preferably at least 95% homology to hAM₁₅₋₅₂, wherein X₁₁ is selected from R, W, or Cit; X₁₇ is selected as Y, W, T, Q, M, I, H, F, A, R, or K; X₄ is selected from F, W, M, I, or C; X₃₇ is selected from G, Y, W, P, H, or F; X₃₈ is selected from Hyp, Y, W, M, or F; and further wherein at least one of the positions X₄, X₃₇ or X₃₈ is not the amino acid present in hAM₁₅₋₅₂ (SEQ ID NO: 1) in said position.

In any of the above-mentioned embodiments, X₁₁ is most preferably R, and/or X₁₇ is most preferably Y, W, or H. Furthermore, in any of the above-mentioned embodiments, most preferably at least two of the positions X₄, X₃₇ or X₃₈ are not the amino acid present in hAM₁₅₋₅₂ (SEQ ID NO: 1) in said position.

In a more highly preferred embodiment, the invention relates to an hAM₁₅₋₅₂ analogue with the sequence SEQ ID NOs: 3-360 or a derivative thereof with at least 50% homology to hAM₁₅₋₅₂, such as at least 55% homology to hAM₁₅₋₅₂, such as at least 60% homology to hAM₁₅₋₅₂, such as at least 65% homology to hAM₁₅₋₅₂, preferably at least 70% homology to hAM₁₅₋₅₂, such as at least 75% homology to hAM₁₅₋₅₂, more preferably at least 80 homology to hAM₁₅₋₅₂, such as at least 90% homology to hAM₁₅₋₅₂ (SEQ ID NO: 1), wherein, in the derivative, X₁₁ is selected from R, W, or Cit; X₁₇ is selected as Y, W, T, Q, M, I, H, F, A, R, or K; X₄ is selected from F, Y, W, T, M, I, A, or C; X₃₇ is selected from G, Y, S, W, T, Q, P, Mv, I, H, F, E, A, R, C, or K; X₃₈ is selected from Hyp, Y, W, T, Q, P, M, I, H, F, E, A, R, or K; and further wherein at least one of the positions X₄, X₃₇ or X₃₈ is not the amino acid present in hAM₁₅₋₅₂ (SEQ ID NO: 1) in said position.

In yet a more highly preferred embodiment, the invention relates to an hAM₁₅₋₅₂ analogue with the sequence SEQ ID NOs: 3-360 or a derivative thereof with at least 90% homology (identity) to any one of SEQ ID NO: 3-360, preferably at least 95% homology to any one of SEQ ID NO: 3-360, most preferably at least 97 homology to anyone of SEQ ID NOs: 3-360, with the proviso that in the derivative, X₁₁ is selected from R, W, or Cit; X₁₇ is selected as Y, W, T, Q, M, I, H, F, A, R, or K; X₄ is selected from F, Y, W, T, M, I, A or C; X₃₇ is selected from G, Y, S, W, T, Q, P, M, I, H, F, E, A, R, C, or K; X₃₈ is selected from Hyp, Y, W, T, Q, P, M, I, H, F, E, A, R, or K; and further wherein at least one of the positions X₄, X₃₇ or X₃₈ is not the amino acid present in hAM₁₅₋₅₂ (SEQ ID NO: 1) in said position.

In any of the above-mentioned embodiments, X₄ is preferably selected as F, W, M, I, or C; X₁₁ is preferably selected as R; X₃₇ is preferably selected as G, Y, W, P, H, or F; X₃₈ is preferably selected as Hyp, Y, W, M, or F; X₁₇ is preferably selected as W or H. Furthermore, in any of the above-mentioned embodiments, most preferably at least two of the positions X₄, X₃₇ or X₃₈ is not the amino acid present in hAM₁₅₋₅₂ (SEQ ID NO: 1) in said position.

In the most preferred embodiment, the invention relates to an hAM₁₅₋₅₂ analogue with the sequence SEQ ID NOs: 3-360 or a derivative thereof with at least 90% homology (identity) to anyone of SEQ ID NOs: 3-360, preferably at least 95% homology to anyone of SEQ ID NOs: 3-360, most preferably at least 97% homology to anyone of SEQ ID NOs: 3-360, with the proviso that a derivative thereof has the same amino acids in positions X₄, X₁₁, X₃₇, and X₃₈, preferably the same amino acids in position X₄, X₁₁, X₁₇, X₃₇, and X₃₈, as the respective hAM₁₅₋₅₂ analogue of SEQ ID NO: 3-360 from which it is derived.

Medical Use

In a third aspect, the invention relates to hAM₁₅₋₅₂ analogues according to the first and/or second aspect for use as a medicament. More particularly, the third aspect of the invention relates to hAM₁₅₋₅₂ analogues according to the first and/or second aspect for use in treating, preventing or ameliorating a variety of diseases, disorders or conditions, such as but not limited to excess food intake, excess body weight, obesity, Binge eating disorder, Prader-Willi syndrome, dyslipidemia, metabolic diseases/disorders, diabetes I or II, impaired glucose tolerance, insulin resistance syndrome and/or NASH. A preferred embodiment relates to one or more hAM₁₅₋₅₂ analogue(s) for use in the treatment of obesity, diabetes, NASH or combinations thereof.

Pharmaceutical Compositions

In a fourth aspect, the invention relates to pharmaceutical compositions comprising one or more of the hAM₁₅₋₅₂ analogues according to the first and/or second aspect and their medical use(s) in treating, preventing, or ameliorating a variety of diseases, disorders or conditions according to the third aspect. The pharmaceutical compositions may comprise a pharmaceutically acceptable carrier (vehicle) and/or one or more excipient(s) in accordance with conventional techniques in the art, such as those disclosed in ‘Remington: Essentials of Pharmaceutics’, Ed. by Linda A. Felton, Pharmaceutical press 2012.

Suitable formulations include but are not limited to tablets, pills, capsules, emulsions, suspensions, sustained release, or solutions. It should be appreciated that different routes of administration may be used depending on the choice of formulation and chemical and/or metabolic stability of the polypeptides. Such administration routes may include but are not limited to oral administration, parenteral administration (intravenous (IV), subcutaneous (SC), intradermal (ID) and intramuscular (IM)), or inhalation. In a preferred embodiment of the invention, the administration route is parental administration. In an even more preferred embodiment, the administration route is subcutaneous.

Method of Treatment

In a fifth aspect, the invention relates to a method of treating a human or animal subject with one or more hAM₁₅₋₅₂ analogue(s) according to the first and/or second aspect, wherein the human or animal subject is diagnosed with or suffering from one or more of the diseases, disorders according to the third aspect. The method involves administering one or more compounds according to a first and/or second aspect in an effective therapeutic amount to treat diseases, disorders or conditions mentioned in the third aspect. In some instances, a human or animal subject may benefit more from an hAM₁₅₋₅₂ analogue having potency on both hAMY3R/hAM1R, e.g. when the patient is in high risk of cardiovascular disease and cardioprotective effects are desired. Thus, in an embodiment, the human or animal subject is treated with one or more hAM₁₅₋₅₂ analogue(s) having mixed hAMY3R/hAM1R potency, such as those exemplified in SEQ ID: 361-392. Likewise, in some instances a human or animal subject may benefit most from a selective hAMY3R hAM₁₅₋₅₂ analogue. Thus, in an embodiment, the human or animal subject is treated with one or more selective hAMY3R hAM₁₅₋₅₂ analogue(s), such as those exemplified in SEQ ID: 3-360.

### EXAMPLES

General Protocols for Synthesis of the Hybrid Polypeptides

The peptides were synthesized using a SyroII fully automated parallel peptide synthesizer (MultiSynTech GmbH, Germany), equipped with heating block, on Tentage) S RAM with a loading of 0.23-0.25 mmol/g (Rapp polymer GmbH, Germany). N^{α}-Fmoc deprotection was performed in two stages by treating the resin with 40 piperidine/DMF (0.2 M HOBt (1-hydroxybenzotriazole)) for 3 min at 45° C. followed by 20% piperidine/DMF (0.1 M HOBt) for 7-12 min at 75° C. Except Asp, Cys and His residues which were N^{α}-Fmoc deprotections at room temperature; i.e. 40% piperidine/DMF (0.2 M HOBt) for 3 min followed by 20% piperidine/DMF (0.1 M HOBt) for 15 min. The coupling chemistry was DIC (N,N′-diisopropyl-carbodiimide)/Oxyma (ethyl cyano(hydroxyimino)acetate) in DMF using amino acid solutions of 0.5 M in DMF and a molar excess of 6-fold. Standard Fmoc protected amino acids were used. Coupling conditions was single or double couplings for 15 min at 75° C. Except His and Cys residues, which were double coupled for 15 min at 50° C. The Fmoc-amino acids were dissolved at 0.5 M in DMF containing 0.5 M Oxyma, except His which was dissolved in NMP. The resin was washed 4× with NMP after N^{α}-Fmoc deprotection and 3× after couplings.

The disulfide bridge was formed on the resin by using Trityl (Trt) as the protecting group for cysteine and treating with 1% iodine in 75% HFIP (Hexafluoroisopropanol) in DCM for 1 min. The resin was washed 3× with 75% HFIP in DCM followed by 4×DCM.

After synthesis, the resin was washed with DCM and dried, and the polypeptide was cleaved from the resin by a 35 min treatment with TFA (trifluoroacetic acid)/TES (triethylsilane)/water (95/2.5/2.5) at 42° C., followed by precipitation with 4 volumes of cold diethyl ether, further washing with diethyl ether and left to dry.

The peptides were characterized by LC-MS (Waters, Denmark) and quantified by LC-CAD (ThermoFisher scientific, Denmark). Finally, the peptides were freeze dried to give a white powder using a Telstar benchtop freeze drier.

General Protocols for cAMP Assays for Measuring In Vitro Receptor Activation hAMY₃-R:

Cells stably overexpressing the hAMY3 receptor were obtained from Ogeda (now Astellas Pharma), sub-cloned and a monoclonal cell-line with an appropriate assay-window was expanded, aliquoted and frozen. An aliquot was thawed and plated in DPBS with 0.05% casein and 0.5 mM IBMX as 2000 cells/well in a 384-well format.

The cells were then immediately stimulated for 30 min at room temperature with graded doses of test compound using human amylin (Bachem, cat no. H-7905) as a positive control. cAMP accumulation was measured using a Cisbio assay for Gs coupled receptors (cat. no. 62AM4PEC), where the assay reagents were added as per the manufacturer's instructions and time-resolved fluorescence energy transfer recorded after one hour.

hAM1-R:

Cells stably overexpressing the hAM1 receptor were obtained from Ogeda (now Astellas Pharma), sub-cloned and a monoclonal cell-line with an appropriate assay-window was expanded, aliquoted and frozen. An aliquot was thawed and plated in DPBS with 0.05% casein and 0.5 mM IBMX as 8000 cells/well in a 384-well format. The cells were then immediately stimulated for 30 min at room temperature with graded doses of test compound using human adrenomedullin 1-52 (Bachem, cat no. H-2932) as a positive control. cAMP accumulation was measured using a Cisbio assay for Gs coupled receptors (cat. no. 62AM4PEC), where the assay reagents were added as per the manufacturer's instructions and time-resolved fluorescence energy transfer recorded after one hour.

General Protocols for Determination of Physical Stability of Peptide Analogues

Peptides were dissolved in buffers (50 mM sodium acetate at pH 4 or 50 mM sodium phosphate at pH 7) and incubated for one hour. The samples were then divided into two replicates of 80 μl in a black 384 well plate (μ-clear, Greiner Bio-One) and mixed with Thioflavin T (ThT) to a final concentration of 4 pM. The plate was sealed and placed in a plate reader (CLARIOstar, BMG) where the temperature was set at 40° C. during the course of the experiment. The samples were further stressed by shaking the plate at 700 rpm (linear) for five minutes before every measurement. The fluorescence was measured every 10 min for four days by exciting the ThT at 450 nm and measuring the emission at 480 nm. For each peptide, the ThT signal over time was smoothened using Local Polynomial Regression fitting (LOESS) as implemented in the statistical programming environment R. For the smoothened data, the maximum ThT signal was normalized in percent between the maximum ThT signal for hAMY and the buffer background. Thus, high values indicate fibrillation properties similar to hAMY while low values indicate no fibrillation.

General Protocols for Acute Food Intake Study

The effect on cumulative food intake was measured in in male Sprague Dawley rats (6 weeks of age, Taconic, Denmark) following a single dose using a fully automated food intake monitoring system (HM-2; MBRose ApS, Faaborg, Denmark), allowing for advanced synchronous real-time monitoring of food intake behaviour of individual animals. The animals were housed in groups of 4 in a light-, temperature-, and humidity-controlled room (a 12/12 LD cycle, lights on at 02:00 AM; 22±2° C.; 50% relative humidity). The mice had ad libitum access to regular chow diet (Altromin 1324, Brogaarden A/S, Lynge, Denmark) and domestic quality tap water. Mice arrived at day 7, and a minimum of 5 days of habituation to the system was allowed prior to beginning of the study. During these days, the animals were handled daily to accustom them to the experimental paradigm. On the day of dosing, the animals were randomized into groups according to body weight. Animals were fasted for four hours prior to dosing. Animals were dosed SC 30 or 180 min prior to lights out, and food intake data were collected automatically for a total of 48 h post-dosing with automatic food recordings every 5 min.

General Protocols for Chronic Diet-Induced Obesity (DIO) Study

Sprague-Dawley rats (Taconic, Denmark) arrived at the age of 5 weeks. After one week of acclimation, they were evenly grouped based on their baseline body weight and offered ad libitum access a two-choice diet consisting of chow pellet (Altromin #1324, Brogaarden, Lynge, Denmark) and a high palatable high sugar/fat diet (1:1:1 mixture of Nutella (Ferrero, Alba, Italy), peanut butter (PCD, Rotterdam, Netherlands) and powdered standard chow (Altromin #1324, Brogaarden, Lynge, Denmark); 29.3% fat, 33.2% carbohydrate, and 18% protein) for up to 36 weeks. The rats were pair-housed throughout the study under controlled environmental conditions (12-hour light/dark cycle, lights off at 15:00; 22±1° C.; 50±10% relative humidity). Body weight, food and water intake were monitored daily during the entire period of the study. One day before the experiment, the rats were randomized according to body weight into experimental groups (n=9-10), which received compound or vehicle SC. QD (once daily) for 28 days.

### Example 1

EC₅₀ values at hAMY3R and hAM1R were measured according to the general protocol above. Percent homology (identity) for the hAM₁₅₋₅₂ analogues were calculated in comparison to hAM₁₅₋₅₂. The results are summarized in Table 2 below.

TABLE 2
Table 2 shows that hAM₁₅₋₅₂ analogues (SEQ ID NO: 3-392) with
improved hAMY3R potency compared to hAM₁₅₋₅₂ may be
obtained by changing the amino acid K present in
hAM₁₅₋₅₂ in position X₁₁ into R, W or Cit. Table 2
further shows that highly selective (hAMY3R-EC₅₀ ≤ 250 pM and an
hAM1R-EC₅₀ ≥ 25 nM) hAMY3R hAM₁₅₋₅₂ analogues (SEQ ID
NO: 3-360) may be obtained by abolishing or reducing the hAM1R
potency using the positions X₄, X₃₇ and/or X₃₈. All lipidations
at position 1 were performed at the N-terminal and the lipidation
in the other positions were performed at an epsilon N in Lys.

| | hAMY3R | hAM1R | identity | | Lipidation |
|---|---|---|---|---|---|
| ID | EC₅₀ (nM) | EC₅₀ (nM) | (% ADM)* | Lipidation | position |
| 1 | 1.274 | 1.113 | 100.0 | None | NA |
| 2 | 0.01 | >5000.000 | 18.4 | None | NA |
| 3 | 0.006 | 123.548 | 73.7 | None | NA |
| 4 | 0.006 | >5000.000 | 63.2 | C20DA- | 1 |
| | | | | yGlu | |
| 5 | 0.006 | >5000.000 | 63.2 | C20DA- | 1 |
| | | | | yGlu | |
| 6 | 0.006 | >5000.000 | 63.2 | C20DA- | 36 |
| | | | | yGlu-OEG- | |
| | | | | OEG | |
| 7 | 0.006 | >5000.000 | 63.2 | None | NA |
| 8 | 0.006 | >5000.000 | 44.7 | None | NA |
| 9 | 0.006 | >5000.000 | 44.7 | None | NA |
| 10 | 0.007 | 551.970 | 68.4 | None | NA |
| 11 | 0.007 | >5000.000 | 63.2 | C20DA- | 1 |
| | | | | yGlu | |
| 12 | 0.007 | >5000.000 | 60.5 | C20DA- | 27 |
| | | | | yGlu-OEG- | |
| | | | | OEG | |
| 13 | 0.007 | >5000.000 | 63.2 | None | NA |
| 14 | 0.007 | >5000.000 | 63.2 | None | NA |
| 15 | 0.007 | >5000.000 | 50.0 | None | NA |
| 16 | 0.008 | 125.467 | 78.9 | None | NA |
| 17 | 0.008 | 294.942 | 71.1 | None | NA |
| 18 | 0.008 | 817.242 | 68.4 | None | NA |
| 19 | 0.008 | >5000.000 | 28.9 | None | NA |
| 20 | 0.008 | >5000.000 | 71.1 | None | NA |
| 21 | 0.008 | >5000.000 | 63.2 | C20DA- | 1 |
| | | | | yGlu | |
| 22 | 0.008 | >5000.000 | 63.2 | C20DA- | 1 |
| | | | | yGlu | |
| 23 | 0.008 | >5000.000 | 63.2 | None | NA |
| 24 | 0.008 | >5000.000 | 63.2 | None | NA |
| 25 | 0.008 | >5000.000 | 63.2 | None | NA |
| 26 | 0.008 | >5000.000 | 63.2 | None | NA |
| 27 | 0.008 | >5000.000 | 63.2 | None | NA |
| 28 | 0.008 | >5000.000 | 65.8 | None | NA |
| 29 | 0.008 | >5000.000 | 55.3 | None | NA |
| 30 | 0.008 | >5000.000 | 63.2 | None | NA |
| 31 | 0.008 | >5000.000 | 60.5 | None | NA |
| 32 | 0.008 | >5000.000 | 50.0 | None | NA |
| 33 | 0.008 | >5000.000 | 52.6 | None | NA |
| 34 | 0.009 | 54.627 | 73.7 | None | NA |
| 35 | 0.009 | >5000.000 | 63.2 | C20DA- | 1 |
| | | | | yGlu | |
| 36 | 0.009 | >5000.000 | 63.2 | C20DA- | 3 |
| | | | | yGlu-OEG- | |
| | | | | OEG | |
| 37 | 0.010 | >5000.000 | 63.2 | C20DA- | 1 |
| | | | | yGlu | |
| 38 | 0.010 | >5000.000 | 63.2 | C20DA- | 1 |
| | | | | yGlu | |
| 39 | 0.010 | >5000.000 | 65.8 | C20DA- | 1 |
| | | | | yGlu | |
| 40 | 0.010 | >5000.000 | 60.5 | C20DA- | 25 |
| | | | | yGlu-OEG- | |
| | | | | OEG | |
| 41 | 0.010 | >5000.000 | 60.5 | C20DA- | 26 |
| | | | | yGlu-OEG- | |
| | | | | OEG | |
| 42 | 0.010 | >5000.000 | 63.2 | None | NA |
| 43 | 0.010 | >5000.000 | 63.2 | None | NA |
| 44 | 0.010 | >5000.000 | 63.2 | None | NA |
| 45 | 0.011 | 50.470 | 86.8 | None | NA |
| 46 | 0.011 | 78.480 | 86.8 | None | NA |
| 47 | 0.011 | 500.000 | 63.2 | C20DA- | 1 |
| | | | | yGlu | |
| 48 | 0.011 | 760.539 | 63.2 | C20DA- | 1 |
| | | | | yGlu | |
| 49 | 0.011 | 2981.000 | 81.6 | None | NA |
| 50 | 0.011 | >5000.000 | 65.8 | C20DA- | 1 |
| | | | | yGlu | |
| 51 | 0.011 | >5000.000 | 63.2 | C20DA- | 1 |
| | | | | yGlu | |
| 52 | 0.011 | >5000.000 | 63.2 | None | NA |
| 53 | 0.011 | >5000.000 | 63.2 | None | NA |
| 54 | 0.012 | 46.194 | 81.6 | None | NA |
| 55 | 0.012 | 63.776 | 63.2 | C20DA- | 1 |
| | | | | yGlu | |
| 56 | 0.012 | 103.250 | 73.7 | None | NA |
| 57 | 0.012 | 590.397 | 63.2 | C20DA- | 1 |
| | | | | yGlu | |
| 58 | 0.012 | >5000.000 | 21.1 | None | NA |
| 59 | 0.012 | >5000.000 | 26.3 | None | NA |
| 60 | 0.012 | >5000.000 | 68.4 | C20DA- | 1 |
| | | | | yGlu | |
| 61 | 0.012 | >5000.000 | 63.2 | C20DA- | 1 |
| | | | | yGlu | |
| 62 | 0.012 | >5000.000 | 63.2 | C20DA- | 1 |
| | | | | yGlu | |
| 63 | 0.012 | >5000.000 | 63.2 | C20DA- | 1 |
| | | | | yGlu | |
| 64 | 0.012 | >5000.000 | 63.2 | None | NA |
| 65 | 0.012 | >5000.000 | 57.9 | None | NA |
| 66 | 0.012 | >5000.000 | 60.5 | None | NA |
| 67 | 0.013 | 116.497 | 71.1 | C20DA- | 1 |
| | | | | yGlu | |
| 68 | 0.013 | 254.722 | 68.4 | C20DA- | 1 |
| | | | | yGlu | |
| 69 | 0.013 | 680.331 | 60.5 | C20DA- | 1 |
| | | | | yGlu | |
| 70 | 0.013 | 2981.000 | 84.2 | None | NA |
| 71 | 0.013 | >5000.000 | 60.5 | C20DA- | 1 |
| | | | | yGlu | |
| 72 | 0.013 | >5000.000 | 63.2 | C20DA- | 1 |
| | | | | yGlu | |
| 73 | 0.013 | >5000.000 | 63.2 | C20DA- | 1 |
| | | | | yGlu | |
| 74 | 0.013 | >5000.000 | 63.2 | C20DA- | 14 |
| | | | | yGlu-OEG- | |
| | | | | OEG | |
| 75 | 0.013 | >5000.000 | 65.8 | None | NA |
| 76 | 0.014 | >5000.000 | 13.2 | C20DA- | 1 |
| | | | | yGlu | |
| 77 | 0.014 | >5000.000 | 65.8 | C20DA- | 1 |
| | | | | yGlu | |
| 78 | 0.014 | >5000.000 | 60.5 | C20DA- | 1 |
| | | | | yGlu | |
| 79 | 0.014 | >5000.000 | 60.5 | C20DA- | 1 |
| | | | | yGlu | |
| 80 | 0.015 | 44.303 | 15.8 | C20DA- | 1 |
| | | | | yGlu | |
| 81 | 0.015 | 138.500 | 84.2 | None | NA |
| 82 | 0.015 | 612.158 | 63.2 | C20DA- | 1 |
| | | | | yGlu | |
| 83 | 0.015 | 815.473 | 63.2 | C20DA- | 1 |
| | | | | yGlu | |
| 84 | 0.015 | 2981.000 | 81.6 | None | NA |
| 85 | 0.015 | >5000.000 | 63.2 | C20DA- | 1 |
| | | | | yGlu | |
| 86 | 0.015 | >5000.000 | 60.5 | C20DA- | 1 |
| | | | | yGlu | |
| 87 | 0.015 | >5000.000 | 60.5 | C20DA- | 1 |
| | | | | yGlu | |
| 88 | 0.015 | >5000.000 | 60.5 | C20DA- | 1 |
| | | | | yGlu | |
| 89 | 0.015 | >5000.000 | 60.5 | C20DA- | 20 |
| | | | | yGlu-OEG- | |
| | | | | OEG | |
| 90 | 0.016 | 58.530 | 81.6 | None | NA |
| 91 | 0.016 | 839.591 | 63.2 | C20DA- | 1 |
| | | | | yGlu | |
| 92 | 0.016 | 932.795 | 60.5 | C20DA- | 1 |
| | | | | yGlu | |
| 93 | 0.016 | >5000.000 | 28.9 | None | NA |
| 94 | 0.016 | >5000.000 | 60.5 | C20DA- | 1 |
| | | | | yGlu | |
| 95 | 0.016 | >5000.000 | 60.5 | C20DA- | 1 |
| | | | | yGlu | |
| 96 | 0.016 | >5000.000 | 60.5 | C20DA- | 1 |
| | | | | yGlu | |
| 97 | 0.016 | >5000.000 | 63.2 | C20DA- | 1 |
| | | | | yGlu | |
| 98 | 0.017 | 66.880 | 86.8 | None | NA |
| 99 | 0.017 | 364.198 | 71.1 | C20DA- | 1 |
| | | | | yGlu | |
| 100 | 0.017 | >5000.000 | 60.5 | C20DA- | 1 |
| | | | | yGlu | |
| 101 | 0.017 | >5000.000 | 63.2 | None | NA |
| 102 | 0.018 | >5000.000 | 71.1 | None | NA |
| 103 | 0.018 | >5000.000 | 63.2 | C20DA- | 1 |
| | | | | yGlu | |
| 104 | 0.018 | >5000.000 | 63.2 | C20DA- | 1 |
| | | | | yGlu | |
| 105 | 0.018 | >5000.000 | 60.5 | C20DA- | 1 |
| | | | | yGlu | |
| 106 | 0.018 | >5000.000 | 60.5 | C20DA- | 1 |
| | | | | yGlu | |
| 107 | 0.019 | 646.950 | 60.5 | C20DA- | 1 |
| | | | | yGlu | |
| 108 | 0.019 | >5000.000 | 60.5 | C20DA- | 1 |
| | | | | yGlu | |
| 109 | 0.020 | 2981.000 | 84.2 | None | NA |
| 110 | 0.020 | >5000.000 | 63.2 | C20DA- | 1 |
| | | | | yGlu | |
| 111 | 0.021 | 2981.000 | 89.5 | None | NA |
| 112 | 0.021 | >5000.000 | 60.5 | C20DA- | 1 |
| | | | | yGlu | |
| 113 | 0.022 | 2981.000 | 89.5 | None | NA |
| 114 | 0.022 | >5000.000 | 63.2 | None | NA |
| 115 | 0.023 | 129.800 | 84.2 | None | NA |
| 116 | 0.024 | 48.170 | 89.5 | None | NA |
| 117 | 0.024 | 51.555 | 78.9 | None | NA |
| 118 | 0.024 | 2981.000 | 84.2 | None | NA |
| 119 | 0.024 | >5000.000 | 65.8 | C20DA- | 1 |
| | | | | yGlu | |
| 120 | 0.025 | >5000.000 | 57.9 | C20DA- | 1 |
| | | | | yGlu | |
| 121 | 0.026 | 2981.000 | 84.2 | None | NA |
| 122 | 0.026 | >5000.000 | 60.5 | C20DA- | 1 |
| | | | | yGlu | |
| 123 | 0.027 | 31.653 | 73.7 | None | NA |
| 124 | 0.027 | 34.840 | 86.8 | None | NA |
| 125 | 0.027 | 2981.000 | 86.8 | None | NA |
| 126 | 0.027 | >5000.000 | 63.2 | C20DA- | 1 |
| | | | | AMCHC- | |
| | | | | yGlu-OEG- | |
| | | | | OEG | |
| 127 | 0.028 | 107.400 | 92.1 | None | NA |
| 128 | 0.028 | 130.300 | 34.2 | None | NA |
| 129 | 0.029 | 366.900 | 89.5 | None | NA |
| 130 | 0.030 | >5000.000 | 57.9 | C20DA- | 1 |
| | | | | yGlu | |
| 131 | 0.031 | 33.760 | 92.1 | None | NA |
| 132 | 0.031 | 2981.000 | 89.5 | None | NA |
| 133 | 0.031 | >5000.000 | 57.9 | C20DA- | 1 |
| | | | | yGlu | |
| 134 | 0.032 | 2981.000 | 86.8 | None | NA |
| 135 | 0.033 | 68.400 | 86.8 | None | NA |
| 136 | 0.033 | >5000.000 | 60.5 | C20DA- | 17 |
| | | | | yGlu-OEG- | |
| | | | | OEG | |
| 137 | 0.034 | >5000.000 | 57.9 | C20DA- | 1 |
| | | | | yGlu | |
| 138 | 0.035 | 2981.000 | 86.8 | None | NA |
| 139 | 0.035 | 2981.000 | 84.2 | None | NA |
| 140 | 0.036 | 31.400 | 92.1 | None | NA |
| 141 | 0.036 | 258.400 | 89.5 | None | NA |
| 142 | 0.036 | 2981.000 | 84.2 | None | NA |
| 143 | 0.038 | 2981.000 | 84.2 | None | NA |
| 144 | 0.038 | >5000.000 | 57.9 | C20DA- | 1 |
| | | | | yGlu | |
| 145 | 0.039 | 2981.000 | 89.5 | None | NA |
| 146 | 0.040 | 2981.000 | 92.1 | None | NA |
| 147 | 0.040 | 3108.878 | 86.8 | None | NA |
| 148 | 0.040 | >5000.000 | 26.3 | None | NA |
| 149 | 0.040 | >5000.000 | 28.9 | None | NA |
| 150 | 0.040 | >5000.000 | 89.5 | None | NA |
| 151 | 0.041 | 44.920 | 84.2 | None | NA |
| 152 | 0.041 | 212.700 | 89.5 | None | NA |
| 153 | 0.041 | >5000.000 | 60.5 | C20DA- | 1 |
| | | | | yGlu | |
| 154 | 0.042 | 2981.000 | 97.4 | None | NA |
| 155 | 0.045 | 116.500 | 92.1 | None | NA |
| 156 | 0.047 | 2981.000 | 86.8 | None | NA |
| 157 | 0.047 | 2981.000 | 86.8 | None | NA |
| 158 | 0.048 | 2981.000 | 92.1 | None | NA |
| 159 | 0.048 | >5000.000 | 63.2 | C20DA- | 1 |
| | | | | yGlu | |
| 160 | 0.050 | 2981.000 | 86.8 | None | NA |
| 161 | 0.051 | 2981.000 | 86.8 | None | NA |
| 162 | 0.051 | >5000.000 | 31.6 | None | NA |
| 163 | 0.053 | 2981.000 | 81.6 | None | NA |
| 164 | 0.054 | 106.200 | 94.7 | None | NA |
| 165 | 0.054 | 2981.000 | 86.8 | None | NA |
| 166 | 0.056 | 2981.000 | 84.2 | None | NA |
| 167 | 0.056 | 2981.000 | 92.1 | None | NA |
| 168 | 0.062 | >5000.000 | 92.1 | None | NA |
| 169 | 0.063 | 2981.000 | 94.7 | None | NA |
| 170 | 0.064 | 2981.000 | 84.2 | None | NA |
| 171 | 0.064 | >5000.000 | 86.8 | None | NA |
| 172 | 0.066 | 2981.000 | 86.8 | None | NA |
| 173 | 0.069 | >5000.000 | 86.8 | None | NA |
| 174 | 0.071 | 133.300 | 94.7 | None | NA |
| 175 | 0.074 | 2981.000 | 89.5 | None | NA |
| 176 | 0.076 | 2981.000 | 86.8 | None | NA |
| 177 | 0.077 | 2981.000 | 84.2 | None | NA |
| 178 | 0.080 | 2981.000 | 84.2 | None | NA |
| 179 | 0.082 | 2981.000 | 89.5 | None | NA |
| 180 | 0.082 | >5000.000 | 26.3 | None | NA |
| 181 | 0.082 | >5000.000 | 63.2 | None | NA |
| 182 | 0.085 | 100.100 | 94.7 | None | NA |
| 183 | 0.088 | 2981.000 | 89.5 | None | NA |
| 184 | 0.090 | 4495.564 | 86.8 | None | NA |
| 185 | 0.090 | >5000.000 | 86.8 | None | NA |
| 186 | 0.091 | >5000.000 | 86.8 | None | NA |
| 187 | 0.093 | 2981.000 | 86.8 | None | NA |
| 188 | 0.094 | 47.640 | 86.8 | None | NA |
| 189 | 0.096 | 2981.000 | 89.5 | None | NA |
| 190 | 0.097 | 2981.000 | 89.5 | None | NA |
| 191 | 0.098 | 2981.000 | 89.5 | None | NA |
| 192 | 0.098 | >5000.000 | 86.8 | None | NA |
| 193 | 0.099 | >5000.000 | 84.2 | None | NA |
| 194 | 0.112 | 3526.547 | 86.8 | None | NA |
| 195 | 0.112 | >5000.000 | 60.5 | None | NA |
| 196 | 0.114 | >5000.000 | 84.2 | None | NA |
| 197 | 0.120 | 2981.000 | 86.8 | None | NA |
| 198 | 0.122 | 3962.089 | 86.8 | None | NA |
| 199 | 0.124 | 2981.000 | 89.5 | None | NA |
| 200 | 0.126 | 38.156 | 81.6 | C20DA- | 1 |
| | | | | yGlu | |
| 201 | 0.126 | 4881.061 | 86.8 | None | NA |
| 202 | 0.126 | >5000.000 | 86.8 | None | NA |
| 203 | 0.127 | >5000.000 | 86.8 | None | NA |
| 204 | 0.129 | 2981.000 | 86.8 | None | NA |
| 205 | 0.130 | 3228.702 | 89.5 | None | NA |
| 206 | 0.130 | >5000.000 | 86.8 | None | NA |
| 207 | 0.131 | >5000.000 | 28.9 | None | NA |
| 208 | 0.133 | 242.450 | 73.7 | C20DA- | 1 |
| | | | | yGlu | |
| 209 | 0.136 | 1020.000 | 89.5 | None | NA |
| 210 | 0.137 | >5000.000 | 63.2 | None | NA |
| 211 | 0.151 | 2756.647 | 86.8 | None | NA |
| 212 | 0.153 | >5000.000 | 89.5 | None | NA |
| 213 | 0.153 | >5000.000 | 84.2 | None | NA |
| 214 | 0.157 | 2981.000 | 86.8 | None | NA |
| 215 | 0.161 | >5000.000 | 86.8 | None | NA |
| 216 | 0.164 | 2981.000 | 86.8 | None | NA |
| 217 | 0.167 | >5000.000 | 89.5 | None | NA |
| 218 | 0.169 | >5000.000 | 86.8 | None | NA |
| 219 | 0.171 | 4246.488 | 86.8 | None | NA |
| 220 | 0.182 | >5000.000 | 86.8 | None | NA |
| 221 | 0.184 | >5000.000 | 63.2 | C20DA- | 1 |
| | | | | AMCHC- | |
| | | | | gGlu-OEG- | |
| | | | | OEG- | |
| | | | | K(C20DA- | |
| | | | | AMCHC- | |
| | | | | gGlu-OEG- | |
| | | | | OEG) | |
| 222 | 0.184 | >5000.000 | 86.8 | None | NA |
| 223 | 0.186 | 47.652 | 78.9 | C20DA- | 1 |
| | | | | yGlu | |
| 224 | 0.186 | 2981.000 | 86.8 | None | NA |
| 225 | 0.186 | >5000.000 | 65.8 | C20DA- | 1 |
| | | | | yGlu | |
| 226 | 0.186 | >5000.000 | 86.8 | None | NA |
| 227 | 0.187 | 4570.819 | 84.2 | None | NA |
| 228 | 0.205 | >5000.000 | 86.8 | None | NA |
| 229 | 0.207 | 2981.000 | 89.5 | None | NA |
| 230 | 0.208 | >5000.000 | 89.5 | None | NA |
| 231 | 0.209 | >5000.000 | 65.8 | C20DA- | 1 |
| | | | | yGlu | |
| 232 | 0.213 | >5000.000 | 86.8 | None | NA |
| 233 | 0.220 | >5000.000 | 63.2 | (C20DA- | 1 |
| | | | | OEG-yGlu- | |
| | | | | OEG)₂-K- | |
| 234 | 0.227 | >5000.000 | 71.1 | None | NA |
| 235 | 0.233 | 1416.600 | 86.8 | None | NA |
| 236 | 0.234 | 2239.417 | 86.8 | None | NA |
| 237 | 0.235 | 4245.930 | 86.8 | None | NA |
| 238 | 0.235 | >5000.000 | 86.8 | None | NA |
| 239 | 0.236 | >5000.000 | 86.8 | None | NA |
| 240 | 0.240 | >5000.000 | 89.5 | None | NA |
| 241 | 0.242 | 2291.534 | 86.8 | None | NA |
| 242 | 0.248 | >5000.000 | 86.8 | None | NA |
| 243 | 0.018 | 3336.098 | 63.2 | None | NA |
| 244 | 0.006 | >5000.000 | 44.7 | None | NA |
| 245 | 0.017 | >5000.000 | 44.7 | None | NA |
| 246 | 0.010 | >5000.000 | 63.2 | None | NA |
| 247 | 0.004 | >5000.000 | 50.0 | None | NA |
| 248 | 0.027 | 3587.646 | 71.1 | None | NA |
| 249 | 0.006 | >5000.000 | 63.2 | None | NA |
| 250 | 0.011 | >5000.000 | 63.2 | None | NA |
| 251 | 0.006 | >5000.000 | 63.2 | None | NA |
| 252 | 0.006 | >5000.000 | 65.8 | None | NA |
| 253 | 0.004 | >5000.000 | 60.5 | None | NA |
| 254 | 0.006 | >5000.000 | 63.2 | None | NA |
| 255 | 0.007 | >5000.000 | 65.8 | None | NA |
| 256 | 0.007 | >5000.000 | 50.0 | None | NA |
| 257 | 0.016 | 2037.788 | 73.7 | None | NA |
| 258 | 0.010 | >5000.000 | 63.2 | None | NA |
| 259 | 0.013 | 4324.040 | 86.8 | None | NA |
| 260 | 0.011 | 4163.023 | 86.8 | None | NA |
| 261 | 0.018 | >5000.000 | 81.6 | None | NA |
| 262 | 0.014 | >5000.000 | 63.2 | None | NA |
| 263 | 0.005 | 1283.849 | 81.6 | None | NA |
| 264 | 0.006 | 3469.936 | 73.7 | None | NA |
| 265 | 0.030 | >5000.000 | 63.2 | None | NA |
| 266 | 0.053 | >5000.000 | 60.5 | None | NA |
| 267 | 0.007 | 2490.994 | 84.2 | None | NA |
| 268 | 0.014 | >5000.000 | 65.8 | None | NA |
| 269 | 0.005 | >5000.000 | 81.6 | None | NA |
| 270 | 0.003 | 1877.713 | 81.6 | None | NA |
| 271 | 0.010 | 96.618 | 86.8 | None | NA |
| 272 | 0.009 | >5000.000 | 71.1 | None | NA |
| 273 | 0.021 | >5000.000 | 84.2 | None | NA |
| 274 | 0.019 | 877.430 | 89.5 | None | NA |
| 275 | 0.020 | >5000.000 | 89.5 | None | NA |
| 276 | 0.056 | >5000.000 | 63.2 | None | NA |
| 277 | 0.058 | >5000.000 | 84.2 | None | NA |
| 278 | 0.011 | 154.831 | 89.5 | None | NA |
| 279 | 0.013 | 241.240 | 78.9 | None | NA |
| 280 | 0.024 | >5000.000 | 84.2 | None | NA |
| 281 | 0.034 | >5000.000 | 84.2 | None | NA |
| 282 | 0.143 | 3880.504 | 73.7 | None | NA |
| 283 | 0.035 | >5000.000 | 86.8 | None | NA |
| 284 | 0.028 | 590.750 | 92.1 | None | NA |
| 285 | 0.157 | >5000.000 | 89.5 | None | NA |
| 286 | 0.025 | 138.873 | 92.1 | None | NA |
| 287 | 0.055 | >5000.000 | 89.5 | None | NA |
| 288 | 0.030 | >5000.000 | 86.8 | None | NA |
| 289 | 0.005 | 3502.991 | 86.8 | None | NA |
| 290 | 0.047 | >5000.000 | 84.2 | None | NA |
| 291 | 0.064 | >5000.000 | 89.5 | None | NA |
| 292 | 0.070 | >5000.000 | 84.2 | None | NA |
| 293 | 0.063 | >5000.000 | 89.5 | None | NA |
| 294 | 0.045 | >5000.000 | 92.1 | None | NA |
| 295 | 0.194 | >5000.000 | 86.8 | None | NA |
| 296 | 0.392 | >5000.000 | 89.5 | None | NA |
| 297 | 0.026 | >5000.000 | 84.2 | None | NA |
| 298 | 0.057 | >5000.000 | 86.8 | None | NA |
| 299 | 0.110 | >5000.000 | 86.8 | None | NA |
| 300 | 0.076 | >5000.000 | 92.1 | None | NA |
| 301 | 0.138 | >5000.000 | 86.8 | None | NA |
| 302 | 0.071 | 4332.084 | 81.6 | None | NA |
| 303 | 0.103 | >5000.000 | 86.8 | None | NA |
| 304 | 0.139 | >5000.000 | 84.2 | None | NA |
| 305 | 0.085 | >5000.000 | 92.1 | None | NA |
| 306 | 0.003 | >5000.000 | 44.7 | None | NA |
| 307 | 0.010 | >5000.000 | 44.7 | None | NA |
| 308 | 0.014 | >5000.000 | 63.2 | None | NA |
| 309 | 0.025 | >5000.000 | 63.2 | None | NA |
| 310 | 0.003 | >5000.000 | 50.0 | None | NA |
| 311 | 0.060 | >5000.000 | 78.9 | None | NA |
| 312 | 0.012 | >5000.000 | 71.1 | None | NA |
| 313 | 0.004 | >5000.000 | 63.2 | None | NA |
| 314 | 0.008 | >5000.000 | 63.2 | None | NA |
| 315 | 0.012 | >5000.000 | 63.2 | None | NA |
| 316 | 0.012 | >5000.000 | 65.8 | None | NA |
| 317 | 0.004 | >5000.000 | 60.5 | None | NA |
| 318 | 0.009 | >5000.000 | 63.2 | None | NA |
| 319 | 0.018 | >5000.000 | 65.8 | None | NA |
| 320 | 0.009 | >5000.000 | 63.2 | None | NA |
| 321 | 0.317 | >5000.000 | 63.2 | None | NA |
| 322 | 0.014 | 1390.178 | 86.8 | None | NA |
| 323 | 0.014 | >5000.000 | 81.6 | None | NA |
| 324 | 0.028 | >5000.000 | 63.2 | None | NA |
| 325 | 0.025 | >5000.000 | 63.2 | None | NA |
| 326 | 0.053 | >5000.000 | 81.6 | None | NA |
| 327 | 0.090 | >5000.000 | 73.7 | None | NA |
| 328 | 0.010 | >5000.000 | 63.2 | None | NA |
| 329 | 0.032 | >5000.000 | 63.2 | None | NA |
| 330 | 0.008 | >5000.000 | 65.8 | None | NA |
| 331 | 0.006 | 3566.754 | 84.2 | None | NA |
| 332 | 0.007 | >5000.000 | 81.6 | None | NA |
| 333 | 0.236 | 107.471 | 63.2 | None | NA |
| 334 | 0.013 | >5000.000 | 71.1 | None | NA |
| 335 | 0.015 | >5000.000 | 84.2 | None | NA |
| 336 | 0.029 | 738.478 | 89.5 | None | NA |
| 337 | 0.019 | 1583.677 | 89.5 | None | NA |
| 338 | 0.089 | >5000.000 | 63.2 | None | NA |
| 339 | 0.047 | >5000.000 | 84.2 | None | NA |
| 340 | 0.022 | 671.058 | 89.5 | None | NA |
| 341 | 0.029 | >5000.000 | 84.2 | None | NA |
| 342 | 0.054 | >5000.000 | 84.2 | None | NA |
| 343 | 0.017 | 117.363 | 86.8 | None | NA |
| 344 | 0.121 | >5000.000 | 89.5 | None | NA |
| 345 | 0.051 | 113.116 | 92.1 | None | NA |
| 346 | 0.085 | >5000.000 | 86.8 | None | NA |
| 347 | 0.068 | >5000.000 | 84.2 | None | NA |
| 348 | 0.041 | >5000.000 | 84.2 | None | NA |
| 349 | 0.046 | >5000.000 | 84.2 | None | NA |
| 350 | 0.044 | 1180.470 | 92.1 | None | NA |
| 351 | 0.943 | >5000.000 | 86.8 | None | NA |
| 352 | 0.040 | 1116.41 | 84.2 | None | NA |
| 353 | 0.027 | 2918.91 | 89.5 | None | NA |
| 354 | 0.091 | >5000.000 | 86.8 | None | NA |
| 355 | 0.140 | >5000.000 | 86.8 | None | NA |
| 356 | 0.079 | >5000.000 | 86.8 | None | NA |
| 357 | 0.115 | >5000.000 | 86.8 | None | NA |
| 358 | 0.071 | >5000.000 | 81.6 | None | NA |
| 359 | 0.145 | >5000.000 | 86.8 | None | NA |
| 360 | 0.165 | >5000.000 | 842 | None | NA |
| 361 | 0.006 | 0.913 | 76.3 | None | NA |
| 362 | 0.007 | 18.150 | 86.8 | None | NA |
| 363 | 0.010 | 1.771 | 71.1 | None | NA |
| 364 | 0.010 | 13.585 | 84.2 | None | NA |
| 365 | 0.012 | 1.211 | 86.8 | None | NA |
| 366 | 0.013 | 12.090 | 89.5 | None | NA |
| 367 | 0.014 | 0.563 | 89.5 | None | NA |
| 368 | 0.015 | 0.997 | 89.5 | None | NA |
| 369 | 0.017 | 2.477 | 84.2 | None | NA |
| 370 | 0.017 | 10.304 | 73.7 | C20DA- | 1 |
| | | | | yGlu | |
| 371 | 0.019 | 3.029 | 92.1 | None | NA |
| 372 | 0.020 | 3.941 | 84.2 | C20DA- | 1 |
| | | | | yGlu | |
| 373 | 0.020 | 7.712 | 86.8 | None | NA |
| 374 | 0.021 | 12.000 | 89.5 | None | NA |
| 375 | 0.022 | 0.403 | 92.1 | None | NA |
| 376 | 0.022 | 12.022 | 81.6 | None | NA |
| 377 | 0.024 | 0.243 | 81.6 | None | NA |
| 378 | 0.027 | 0.893 | 76.3 | C20DA- | 1 |
| | | | | yGlu | |
| 379 | 0.029 | 0.458 | 89.5 | None | NA |
| 380 | 0.031 | 0.162 | 86.8 | C20DA- | 1 |
| | | | | yGlu | |
| 381 | 0.032 | 7.608 | 86.8 | None | NA |
| 382 | 0.034 | 5.124 | 92.1 | None | NA |
| 383 | 0.034 | 5.935 | 92.1 | None | NA |
| 384 | 0.036 | 1.090 | 92.1 | None | NA |
| 385 | 0.036 | 7.003 | 89.5 | None | NA |
| 386 | 0.039 | 7.354 | 78.9 | C20DA- | 1 |
| | | | | yGlu | |
| 387 | 0.040 | 0.992 | 94.7 | None | NA |
| 388 | 0.043 | 4.006 | 84.2 | C20DA- | 1 |
| | | | | yGlu | |
| 389 | 0.045 | 1.039 | 71.1 | C20DA- | 1 |
| | | | | yGlu | |
| 390 | 0.048 | 0.889 | 86.8 | C20DA- | 1 |
| | | | | yGlu | |
| 391 | 0.068 | 0.665 | 92.1 | None | NA |
| 392 | 0.113 | 5.034 | 84.2 | C20DA- | 1 |
| | | | | yGlu | |

AMCHC refers to trans-4-(aminomethyl)cyclohexanecarboxylic acid.
OEG refers to 8-amino-3,6-dioxaoctanoic acid or 8Ado.

### Example 2

Fibrillation was determined according to the general protocol for determination of physical stability of peptide analogues. The data for selected hAM₁₅₋₅₂ analogues are summarized in Table 3 below.

TABLE 3
Table 3 shows that the hAM₁₅₋₅₂ analogues according to the
invention maintain the good fibrillation properties from hAM₁₅₋₅₂.
Most of the fibrillation data are comparable to and some even
better than hAM₁₅₋₅₂ (SEQ ID NO: 1) at pH 7 (ThT signal of
2.6%). All of the fibrillation data are far superior compared to the
reference hAMY₁₋₃₇ (SEQ ID NO: 2) (ThT signal on 100%).

| ID | Max ThT signal (%) | identity (% ADM)* |
|---|---|---|
| 1 | 2.600 | 100.0 |
| 2 | 100 | 18.4 |
| 7 | 2.800 | 63.2 |
| 13 | 5.000 | 63.2 |
| 14 | 3.100 | 63.2 |
| 20 | 9.100 | 71.1 |
| 23 | 4.200 | 63.2 |
| 24 | 6.800 | 63.2 |
| 25 | 33.400 | 63.2 |
| 26 | 0.000 | 63.2 |
| 27 | 21.80 | 63.2 |
| 28 | 8.200 | 65.8 |
| 29 | 9.300 | 55.3 |
| 32 | 5.300 | 50.0 |
| 42 | 6.300 | 63.2 |
| 43 | 1.200 | 63.2 |
| 45 | 3.000 | 86.8 |
| 46 | 5.800 | 86.8 |
| 49 | 3.200 | 81.6 |
| 52 | 7.600 | 63.2 |
| 64 | 2.700 | 63.2 |
| 65 | 1.000 | 57.9 |
| 70 | 8.800 | 84.2 |
| 81 | 4.000 | 84.2 |
| 84 | 14.200 | 81.6 |
| 90 | 2.000 | 81.6 |
| 98 | 12.600 | 86.8 |
| 109 | 9.300 | 84.2 |
| 113 | 14.700 | 89.5 |
| 114 | 2.200 | 63.2 |
| 115 | 2.500 | 84.2 |
| 116 | 11.700 | 89.5 |
| 118 | 7.400 | 84.2 |
| 121 | 1.800 | 84.2 |
| 124 | 4.800 | 86.8 |
| 125 | 7.200 | 86.8 |
| 129 | 1.400 | 89.5 |
| 131 | 6.800 | 92.1 |
| 132 | 5.200 | 89.5 |
| 134 | 17.400 | 86.8 |
| 135 | 3.300 | 86.8 |
| 138 | 6.800 | 86.8 |
| 139 | 4.800 | 84.2 |
| 140 | 3.100 | 92.1 |
| 141 | 3.200 | 89.5 |
| 143 | 2.200 | 84.2 |
| 145 | 2.000 | 89.5 |
| 146 | 3.600 | 92.1 |
| 147 | 5.100 | 89.2 |
| 150 | 18.200 | 91.9 |
| 152 | 1.900 | 89.5 |
| 156 | 15.300 | 86.8 |
| 158 | 6.300 | 92.1 |
| 160 | 21.500 | 86.8 |
| 161 | 14.300 | 86.8 |
| 163 | 2.000 | 81.6 |
| 164 | 24.300 | 94.7 |
| 165 | 4.800 | 86.8 |
| 166 | 2.000 | 84.2 |
| 167 | 6.600 | 92.1 |
| 168 | 12.500 | 94.6 |
| 169 | 2.700 | 94.7 |
| 170 | 1.800 | 84.2 |
| 171 | 5.000 | 89.2 |
| 172 | 13.900 | 86.8 |
| 173 | 4.400 | 89.2 |
| 175 | 3.200 | 89.5 |
| 176 | 6.800 | 86.8 |
| 181 | 8.800 | 63.2 |
| 182 | 15.600 | 94.7 |
| 184 | 0.800 | 89.2 |
| 185 | 2.800 | 89.2 |
| 186 | 0.600 | 89.2 |
| 187 | 1.600 | 86.8 |
| 189 | 6.000 | 89.5 |
| 190 | 12.900 | 89.5 |
| 191 | 6.300 | 89.5 |
| 192 | 4.100 | 89.2 |
| 193 | 2.700 | 86.5 |
| 194 | 6.000 | 89.2 |
| 195 | 6.100 | 60.5 |
| 196 | 0.500 | 86.5 |
| 197 | 5.600 | 86.8 |
| 198 | 4.200 | 89.2 |
| 199 | 8.900 | 89.5 |
| 201 | 1.000 | 89.2 |
| 202 | 12.600 | 89.2 |
| 203 | 1.200 | 89.2 |
| 204 | 2.200 | 86.8 |
| 205 | 7.800 | 91.9 |
| 206 | 1.500 | 89.2 |
| 210 | 1.400 | 63.2 |
| 211 | 5.200 | 89.2 |
| 212 | 1.900 | 91.9 |
| 213 | 6.500 | 86.5 |
| 214 | 2.700 | 86.8 |
| 215 | 0.600 | 89.2 |
| 217 | 13.800 | 91.9 |
| 218 | 4.500 | 89.2 |
| 219 | 0.700 | 89.2 |
| 220 | 1.100 | 89.2 |
| 222 | 11.700 | 89.2 |
| 224 | 2.000 | 86.8 |
| 226 | 27.000 | 89.2 |
| 227 | 8.500 | 86.5 |
| 228 | 19.600 | 89.2 |
| 230 | 0.600 | 91.9 |
| 232 | 1.200 | 89.2 |
| 235 | 0.800 | 89.2 |
| 236 | 0.600 | 89.2 |
| 237 | 0.300 | 89.2 |
| 238 | 0.600 | 89.2 |
| 239 | 0.200 | 89.2 |
| 240 | 3.700 | 91.9 |
| 241 | 0.900 | 89.2 |
| 242 | 0.800 | 89.2 |
| 243 | 8.550 | 63.2 |
| 244 | 4.100 | 44.7 |
| 245 | 3.350 | 44.7 |
| 246 | 4.200 | 63.2 |
| 247 | 1.350 | 50.0 |
| 248 | 13.600 | 71.1 |
| 249 | 15.600 | 63.2 |
| 250 | 6.400 | 63.2 |
| 251 | 10.600 | 63.2 |
| 252 | 27.550 | 63.2 |
| 253 | 9.600 | 60.5 |
| 254 | 12.050 | 63.2 |
| 255 | 25.000 | 65.8 |
| 256 | 18.650 | 50.0 |
| 257 | 20.950 | 73.7 |
| 258 | 32.700 | 63.2 |
| 259 | 13.450 | 86.8 |
| 260 | 34.100 | 86.8 |
| 261 | 22.650 | 81.6 |
| 262 | 9.550 | 63.2 |
| 263 | 1.050 | 81.6 |
| 264 | 1.350 | 73.7 |
| 265 | 26.550 | 63.2 |
| 266 | 20.900 | 60.5 |
| 267 | 30.900 | 84.2 |
| 268 | 8.850 | 65.8 |
| 269 | 34.500 | 81.6 |
| 270 | 25.450 | 81.6 |
| 271 | 10.850 | 86.8 |
| 272 | 16.750 | 71.1 |
| 273 | 29.000 | 84.2 |
| 274 | 26.800 | 89.5 |
| 275 | 19.850 | 89.5 |
| 276 | 8.950 | 63.2 |
| 277 | 21.600 | 84.2 |
| 278 | 13.350 | 89.5 |
| 279 | 13.450 | 78.9 |
| 280 | 26.450 | 84.2 |
| 281 | 22.650 | 84.2 |
| 282 | 4.700 | 73.7 |
| 283 | 17.100 | 86.8 |
| 284 | 21.300 | 92.1 |
| 285 | 31.200 | 89.5 |
| 286 | 2.900 | 92.1 |
| 287 | 10.650 | 89.5 |
| 288 | 17.450 | 86.8 |
| 289 | 21.150 | 86.8 |
| 290 | 17.300 | 84.2 |
| 291 | 19.200 | 89.5 |
| 292 | 19.700 | 84.2 |
| 293 | 24.200 | 89.5 |
| 294 | 16.850 | 92.1 |
| 295 | 19.450 | 86.8 |
| 296 | 26.050 | 89.5 |
| 297 | 27.500 | 84.2 |
| 298 | 31.250 | 86.8 |
| 299 | 26.550 | 86.8 |
| 300 | 17.850 | 92.1 |
| 301 | 16.950 | 86.8 |
| 302 | 24.250 | 81.6 |
| 303 | 18.150 | 86.8 |
| 304 | 22.350 | 84.2 |
| 305 | 24.600 | 92.1 |
| 306 | 3.000 | 44.7 |
| 307 | 11.200 | 44.7 |
| 308 | 14.500 | 63.2 |
| 309 | 4.250 | 63.2 |
| 310 | 3.500 | 50.0 |
| 311 | 6.450 | 78.9 |
| 312 | 17.000 | 71.1 |
| 313 | 15.050 | 63.2 |
| 314 | 11.900 | 63.2 |
| 315 | 17.150 | 86.8 |
| 316 | 20.450 | 89.5 |
| 317 | 5.550 | 86.8 |
| 318 | 14.900 | 89.5 |
| 319 | 3.900 | 92.1 |
| 320 | 11.100 | 89.5 |
| 321 | 21.700 | 94.7 |
| 322 | 18.400 | 92.1 |
| 323 | 27.500 | 81.6 |
| 324 | 11.850 | 63.2 |
| 325 | 10.100 | 63.2 |
| 326 | 2.350 | 81.6 |
| 327 | 3.600 | 73.7 |
| 328 | 33.250 | 63.2 |
| 329 | 6.350 | 63.2 |
| 330 | 14.600 | 65.8 |
| 331 | 21.150 | 84.2 |
| 332 | 2.100 | 81.6 |
| 333 | 25.700 | 63.2 |
| 334 | 10.150 | 71.1 |
| 335 | 1.600 | 84.2 |
| 336 | 18.700 | 89.5 |
| 337 | 16.800 | 89.5 |
| 338 | 2.950 | 63.2 |
| 339 | 20.050 | 84.2 |
| 340 | 1.700 | 89.5 |
| 341 | 20.500 | 84.2 |
| 342 | 19.800 | 84.2 |
| 343 | 24.850 | 86.8 |
| 344 | 13.350 | 89.5 |
| 345 | 3.250 | 92.1 |
| 346 | 1.200 | 86.8 |
| 347 | 15.500 | 84.2 |
| 348 | 2.750 | 84.2 |
| 349 | 15.250 | 84.2 |
| 350 | 15.700 | 92.1 |
| 351 | 7.200 | 86.8 |
| 352 | 25.800 | 84.2 |
| 353 | 30.300 | 89.5 |
| 354 | 17.400 | 86.8 |
| 355 | 25.350 | 86.8 |
| 356 | 1.250 | 86.8 |
| 357 | 19.750 | 86.8 |
| 358 | 24.350 | 81.6 |
| 359 | 20.350 | 86.8 |
| 360 | 26.350 | 84.2 |
| 362 | 8.900 | 86.8 |
| 366 | 3.300 | 89.5 |
| 373 | 18.000 | 86.8 |
| 374 | 4.800 | 89.5 |
| 383 | 9.600 | 92.1 |
| 385 | 8.800 | 89.5 |
| 387 | 14.300 | 94.7 |
| 391 | 3.700 | 92.1 |

### Example 3

The effect of selected hAM₁₅₋₅₂ analogues on acute food intake in lean rats were determined. The results are summarized in Table 4 below.

TABLE 4
Table 4 shows that hAM₁₅₋₅₂ analogues have potent anorectic effects
for up to 48 hours following a single SC injection in
lean healthy rats (n = 7-8), thereby providing proof-of-principle.

| | | Average cumulative | % to | Significance |
|---|---|---|---|---|
| ID | Dose | food intake (g) | vehicle | level to Veh |
| NA | Vehicle | 44.4 | 100 | NA |
| 4 | 10 | 26.3 | 59.23 | *** |
| | nmol/kg | | | |
| 4 | 30 | 13.7 | 30.86 | *** |
| | nmol/kg | | | |
| 46 | 10 | 23.3 | 52.48 | *** |
| | nmol/kg | | | |
| 46 | 30 | 10.7 | 24.10 | *** |
| | nmol/kg | | | |
| 60 | 10 | 30.9 | 69.59 | *** |
| | nmol/kg | | | |
| 60 | 30 | 20.3 | 45.72 | *** |
| | nmol/kg | | | |
| NA | Vehicle | 40.7 | 100 | NA |
| 78 | 10 | 13.7 | 33.66 | *** |
| | nmol/kg | | | |
| 69 | 10 | 7.13 | 17.52 | *** |
| | nmol/kg | | | |
| 70 | 10 | 24.9 | 61.18 | *** |
| | nmol/kg | | | |
| 85 | 10 | 18.6 | 45.70 | *** |
| | nmol/kg | | | |
| 124 | 10 | 24.2 | 59.46 | *** |
| | nmol/kg | | | |
| 93 | 10 | 19.1 | 46.93 | *** |
| | nmol/kg | | | |
| NA | Vehicle | 41.9 | 100 | NA |
| 36 | 10 | 23.3 | 55.61 | *** |
| | nmol/kg | | | |
| 6 | 10 | 27.5 | 65.63 | *** |
| | nmol/kg | | | |

Dunnett's test one-factor linear model***: P < 0.001 compared to Vehicle (three independent runs were conducted).

### Example 4

The Effect of chronic treatment of selected hAM₁₅₋₅₂ analogues according to the invention in diet-induced obese (DIO) rats were determined. The results are summarized in Table 5 below.

TABLE 5
Table 5 shows that daily SC injections of SEQ ID NO: 46 or 78 cause a
significant reduction in body weight in diet-induced obese rats (n = 10)
following a 28-day treatment period, thereby providing proof-of-concept.

| | | Average body | % to | Significance |
|---|---|---|---|---|
| ID | Dose | weight day 28 (gram) | vehicle | level to Veh |
| NA | Vehicle | 716 | 100 | NA |
| 46 | 1 | 674 | 94.13 | *** |
| | nmol/kg | | | |
| 46 | 3 | 620 | 86.59 | *** |
| | nmol/kg | | | |
| 46 | 10 | 618 | 86.31 | *** |
| | nmol/kg | | | |
| NA | Vehicle | 776 | 100 | NA |
| 78 | 10 | 687 | 88.53 | *** |
| | nmol/kg | | | |

Dunnett's test one-factor linear model***: P < 0.001 compared to Vehicle (two independent runs were conducted).

### Example 5

Deeper investigation of representative amino acids in the position X₄, X₁₁, X₁₇, X₃₇ and X₃₈ and the corresponding change in potency. The results are summarized in Table 6 below.

TABLE 6
Table 6 shows whether the amino acid change causes increase
(I) in hAMY3R potency, unchanged potency (U), or decreased
potency (D) compared to the reference (R) amino acid.

| | Position | Position | Position | Position | Position |
|---|---|---|---|---|---|
| Amino acid | 4 | 11 | 17 | 37 | 38 |
| Y | U | D | R | I | I |
| W | I | U | I | I | I |
| T | R | D | U | R | U |
| Q | D | D | U | U | U |
| P | D | D | D | I | U |
| M | I | D | U | U | I |
| I | I | D | U | U | U |
| H | D | D | I | I | U |
| F | I | D | U | I | I |
| E | D | D | D | U | U |
| A | U | D | U | U | U |
| R | D | R | U | U | U |
| C | I | D | D | U | N/A |
| K | D | D | U | U | U |
| Hyp | N/A | N/A | N/A | N/A | R |
| Cit | N/A | U | N/A | N/A | N/A |

N/A Not available.

TABLE 7
SEQUENCE LISTING

| ID | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| 1 | G | C | R | F | G | T | C | T | V | Q | K | L | A | H | Q | I | Y | Q | F | T |
| 2 | K | C | N | T | A | T | C | A | T | Q | R | L | A | N | F | L | V | H | S | S |
| 3 | K | C | N | F | A | T | C | T | V | Q | R | L | A | H | Q | I | Y | Q | F | T |
| 4 | K | C | N | T | A | T | C | T | V | D | R | L | A | H | Q | I | Y | Q | F | T |
| 5 | K | C | N | T | A | T | C | T | V | Q | R | L | A | D | Q | I | Y | Q | F | T |
| 6 | K | C | N | T | A | T | C | T | V | Q | R | L | A | E | Q | I | Y | Q | F | T |
| 7 | K | C | N | T | A | T | C | T | V | Q | R | L | A | A | Q | I | Y | Q | F | T |
| 8 | K | C | N | T | S | T | C | T | V | A | R | L | A | D | Q | I | T | Q | F | S |
| 9 | R | C | N | A | S | T | C | T | V | N | R | L | A | D | Q | I | T | Q | F | S |
| 10 | K | C | N | F | A | T | C | T | V | Q | R | L | A | H | Q | I | Y | Q | F | T |
| 11 | K | C | N | T | A | T | C | T | V | Q | R | L | A | H | Q | I | N | Q | F | T |
| 12 | K | C | N | T | A | T | C | T | V | Q | R | L | A | E | Q | I | Y | Q | F | T |
| 13 | K | C | N | T | A | T | C | T | V | Q | R | L | A | H | A | I | Y | Q | F | T |
| 14 | K | C | N | T | A | T | C | T | V | Q | R | L | A | H | Q | I | A | Q | F | T |
| 15 | K | C | N | T | S | T | C | T | V | A | R | L | A | D | Q | I | T | Q | F | S |
| 16 | G | C | R | F | G | T | C | T | V | Q | R | L | A | N | F | L | Y | Q | F | T |
| 17 | K | C | N | F | A | T | C | T | V | Q | R | L | A | H | Q | I | Y | Q | F | T |
| 18 | K | C | N | F | A | T | C | T | V | Q | R | L | A | H | Q | I | Y | Q | F | T |
| 19 | K | C | N | T | A | T | C | A | T | Q | R | L | A | N | F | L | V | H | S | S |
| 20 | K | C | N | T | A | T | C | T | V | Q | R | L | A | H | Q | I | Y | Q | F | T |
| 21 | K | C | N | T | A | T | C | T | V | Q | R | L | A | N | Q | I | Y | Q | F | T |
| 22 | K | C | N | T | A | T | C | T | V | Q | R | L | A | H | Q | I | T | Q | F | T |
| 23 | K | C | N | T | A | T | C | T | V | Q | R | L | A | H | Q | I | Y | Q | F | A |
| 24 | K | C | N | T | A | T | C | T | V | Q | R | L | A | H | Q | I | Y | Q | F | T |
| 25 | K | C | N | T | A | T | C | T | V | Q | R | L | A | H | Q | I | Y | Q | F | T |
| 26 | K | C | N | T | A | T | C | T | V | Q | R | L | A | H | Q | I | Y | Q | F | T |
| 27 | K | C | N | T | A | T | C | T | V | Q | R | L | A | H | Q | I | Y | Q | F | T |
| 28 | K | C | N | T | A | T | C | T | V | Q | R | L | A | H | Q | I | Y | Q | F | T |
| 29 | K | C | N | T | A | T | C | T | V | Q | R | L | A | H | Q | I | Y | Q | Q | F |
| 30 | K | C | N | T | A | T | C | T | V | Q | R | L | A | H | Q | I | Y | Q | F | T |
| 31 | K | C | N | T | A | T | C | T | V | Q | R | L | A | H | Q | I | Y | Q | F | T |
| 32 | R | C | Q | T | S | T | C | T | V | A | R | L | A | E | Q | I | A | Q | Y | T |
| 33 | R | C | Q | A | S | T | C | T | V | A | Cit | L | A | E | Q | I | A | Q | Y | T |
| 34 | K | C | N | T | A | T | C | T | V | Q | R | L | A | H | Q | I | Y | Q | F | T |
| 35 | K | C | N | T | A | T | C | T | V | Q | R | L | A | H | Q | I | Q | Q | F | T |
| 36 | K | C | K | T | A | T | C | T | V | Q | R | L | A | E | Q | I | Y | Q | F | T |
| 37 | K | C | N | T | A | T | C | T | V | A | R | L | A | H | Q | I | Y | Q | F | T |
| 38 | K | C | N | T | A | T | C | T | V | N | R | L | A | H | Q | I | Y | Q | F | T |
| 39 | R | C | N | T | A | T | C | T | V | Q | R | L | A | H | Q | I | Y | Q | F | T |
| 40 | K | C | N | T | A | T | C | T | V | Q | R | L | A | E | Q | I | Y | Q | F | T |
| 41 | K | C | N | T | A | T | C | T | V | Q | R | L | A | E | Q | I | Y | Q | F | T |
| 42 | K | C | N | T | A | T | C | T | V | Q | R | L | A | H | Q | I | Y | A | F | T |
| 43 | K | C | N | T | A | T | C | T | V | Q | R | L | A | H | Q | I | Y | Q | F | T |
| 44 | K | C | N | T | A | T | C | T | V | Q | R | L | A | E | Q | I | Y | Q | F | T |
| 45 | G | C | R | F | G | T | C | T | V | Q | R | L | A | H | Q | I | Y | Q | F | T |
| 46 | G | C | R | F | G | T | C | T | V | Q | R | L | A | H | Q | I | Y | Q | F | T |
| 47 | K | C | N | T | A | T | C | T | V | Q | R | L | A | Y | Q | I | Y | Q | F | T |
| 48 | K | C | N | T | A | T | C | T | V | Q | R | L | A | H | Q | I | Y | Q | Y | T |
| 49 | G | C | R | T | G | T | C | T | V | Q | R | L | A | H | Q | I | Y | Q | F | T |
| 50 | K | C | N | T | S | T | C | T | V | Q | R | L | A | H | Q | I | Y | Q | F | T |
| 51 | K | C | N | T | A | T | C | T | V | Q | R | L | A | E | Q | I | Y | Q | F | T |
| 52 | K | C | N | T | A | T | C | T | V | Q | R | L | A | H | Q | I | Y | Q | F | T |
| 53 | K | C | N | T | A | T | C | T | V | Q | R | L | A | H | Q | I | Y | Q | F | T |
| 54 | G | C | R | F | G | T | C | T | V | Q | R | L | A | H | F | L | Y | Q | F | T |
| 55 | K | C | N | T | A | T | C | T | V | E | R | L | A | H | Q | I | Y | Q | F | T |
| 56 | G | C | R | F | G | T | C | T | V | Q | R | L | A | H | F | L | Y | Q | F | T |
| 57 | K | C | N | T | A | T | C | T | V | Q | R | L | A | H | Q | I | Y | Q | F | S |
| 58 | K | C | N | T | A | T | C | A | T | Q | R | L | A | N | F | L | V | H | S | S |
| 59 | K | C | N | T | A | T | C | A | T | Q | R | L | A | N | F | L | V | H | S | S |
| 60 | K | C | N | T | A | T | C | T | V | Q | R | L | A | H | Q | I | Y | Q | F | T |
| 61 | K | C | N | T | A | T | C | T | V | Q | R | L | A | H | Q | I | A | Q | F | T |
| 62 | K | C | N | T | S | T | C | T | V | Q | R | L | A | H | Q | I | A | Q | F | T |
| 63 | K | C | N | T | A | T | C | T | V | Q | R | L | A | E | Q | I | Y | Q | F | T |
| 64 | K | C | N | T | A | T | C | T | V | Q | R | L | A | H | Q | I | Y | Q | F | T |
| 65 | K | C | N | T | A | T | C | T | V | Q | R | L | A | H | Q | I | Y | — | Q | F |
| 66 | K | C | N | T | A | T | C | T | V | Q | R | L | A | E | Q | I | Y | Q | F | T |
| 67 | K | C | N | F | A | T | C | T | V | Q | R | L | A | H | Q | I | Y | Q | F | T |
| 68 | K | C | N | F | A | T | C | T | V | Q | R | L | A | H | Q | I | Y | Q | F | T |
| 69 | K | C | N | T | A | T | C | T | V | Q | R | L | A | H | Q | I | A | Q | F | T |
| 70 | G | C | N | F | G | T | C | T | V | Q | R | L | A | H | Q | I | Y | Q | F | T |
| 71 | K | C | N | T | A | T | C | T | V | Q | R | L | A | H | Q | I | A | Q | Y | T |
| 72 | K | C | Q | T | A | T | C | T | V | Q | R | L | A | E | Q | I | Y | Q | F | T |
| 73 | K | C | N | T | A | T | C | T | V | Q | R | L | A | E | Q | I | Y | Q | F | T |
| 74 | K | C | N | T | A | T | C | T | V | Q | R | L | A | K | Q | I | Y | Q | F | T |
| 75 | K | C | N | T | A | T | C | T | V | Q | R | L | A | H | Q | I | Y | Q | F | T |
| 76 | R | C | G | T | A | T | C | A | T | E | R | L | A | AAD | F | L | Q | R | S | — |
| 77 | K | C | N | T | A | T | C | T | V | Q | R | L | A | H | Q | I | Y | O | F | T |
| 78 | K | C | N | T | A | T | C | T | V | Q | R | L | A | E | Q | I | A | Q | F | T |
| 79 | K | C | N | T | A | T | C | T | V | Q | R | L | A | H | Q | I | A | Q | F | S |
| 80 | R | C | Q | T | A | T | C | A | T | E | R | L | A | H | F | L | Q | R | S | — |
| 81 | G | C | R | F | G | T | C | T | V | Q | R | L | A | H | Q | I | Y | Q | F | T |
| 82 | K | C | N | T | A | T | C | T | V | Q | R | L | A | H | Q | I | Y | Q | F | T |
| 83 | K | C | N | T | A | T | C | T | V | Q | R | L | A | H | Q | I | Y | Q | F | A |
| 84 | G | C | R | T | G | T | C | T | V | Q | R | L | A | H | Q | I | Y | Q | F | T |
| 85 | K | C | N | T | A | T | C | T | V | Q | R | L | A | H | Q | I | V | Q | F | T |
| 86 | K | C | N | T | A | T | C | T | V | A | R | L | A | H | Q | I | N | Q | F | T |
| 87 | K | C | N | T | A | T | C | T | V | Q | R | L | A | H | Q | I | A | Q | F | T |
| 88 | K | C | N | T | S | T | C | T | V | Q | R | L | A | H | Q | I | A | Q | F | T |
| 89 | K | C | N | T | A | T | C | T | V | Q | R | L | A | E | Q | I | Y | Q | F | K |
| 90 | G | C | R | F | G | T | C | T | V | Q | R | L | A | H | Q | I | Y | Q | F | T |
| 91 | K | C | N | T | A | T | C | T | V | Q | R | L | A | H | Q | I | Y | Q | F | T |
| 92 | K | C | N | T | A | T | C | T | V | Q | R | L | A | H | Q | I | A | Q | F | T |
| 93 | K | C | N | T | A | T | C | A | T | Q | R | L | A | N | F | L | V | H | S | S |
| 94 | K | C | N | T | A | T | C | T | V | Q | R | L | A | H | Q | I | A | Q | F | Y |
| 95 | K | C | N | T | A | T | C | T | V | Q | R | L | A | H | Q | I | A | Q | F | T |
| 96 | K | C | N | T | A | T | C | T | V | Q | R | L | A | E | Q | I | Y | Q | F | T |
| 97 | K | C | A | T | A | T | C | T | V | Q | R | L | A | E | Q | I | Y | Q | F | T |
| 98 | G | C | R | F | G | T | C | T | V | Q | R | L | A | H | Q | I | Y | Q | F | T |
| 99 | K | C | N | T | A | T | C | T | V | Q | R | L | A | H | Q | I | Y | Q | F | T |
| 100 | K | C | N | T | A | T | C | T | V | Q | R | L | A | E | Q | I | Y | Q | F | T |
| 101 | K | C | N | A | A | T | C | T | V | Q | R | L | A | E | Q | I | Y | Q | F | T |
| 102 | K | C | N | T | A | T | C | T | V | Q | R | L | A | H | Q | I | Y | Q | F | T |
| 103 | K | C | N | T | A | T | C | T | V | V | R | L | A | H | Q | I | Y | Q | F | T |
| 104 | K | C | N | T | A | T | C | T | V | Q | R | L | A | H | Q | I | Y | Q | F | Y |
| 105 | K | C | N | T | A | T | C | T | V | Q | R | L | A | H | Q | I | A | Q | F | A |
| 106 | K | C | N | T | A | T | C | T | V | Q | R | L | A | E | Q | I | Y | Q | F | T |
| 107 | K | C | N | T | A | T | C | T | V | A | R | L | A | H | Q | I | A | Q | F | T |
| 108 | K | C | N | T | A | T | C | T | V | Q | R | L | A | E | Q | I | Y | Q | F | T |
| 109 | G | C | R | T | G | T | C | T | V | Q | R | L | A | H | Q | I | Y | Q | F | T |
| 110 | K | C | N | T | A | T | C | T | V | Q | R | L | A | H | Q | I | E | Q | F | T |
| 111 | G | C | R | F | G | T | C | T | V | Q | R | L | A | H | Q | I | Y | Q | F | T |
| 112 | K | C | N | T | A | T | C | T | V | D | R | L | A | H | Q | I | A | Q | F | T |
| 113 | G | C | R | F | G | T | C | T | V | Q | R | L | A | H | Q | I | Y | Q | F | T |
| 114 | K | C | N | T | A | T | C | T | V | Q | R | L | A | H | Q | I | Y | Q | A | T |
| 115 | G | C | R | T | G | T | C | T | V | Q | R | L | A | H | Q | I | Y | Q | F | T |
| 116 | G | C | R | F | G | T | C | T | V | Q | R | L | A | H | Q | I | Y | Q | F | T |
| 117 | G | C | R | F | G | T | C | T | V | Q | R | L | A | H | F | L | Y | Q | F | T |
| 118 | G | C | R | T | G | T | C | T | V | Q | R | L | A | H | Q | I | Y | Q | F | T |
| 119 | K | C | N | T | Aib | T | C | T | V | Q | R | L | A | H | Q | I | Y | Q | F | T |
| 120 | K | C | N | T | A | T | C | T | V | Q | R | L | A | H | Q | I | A | Q | F | T |
| 121 | G | C | R | T | G | T | C | T | V | Q | R | L | A | H | Q | I | Y | Q | F | T |
| 122 | K | C | N | T | A | T | C | T | V | Q | R | L | A | E | Q | I | Y | Q | F | T |
| 123 | G | C | R | F | G | T | C | T | V | Q | R | L | A | H | F | L | Y | Q | F | T |
| 124 | G | C | R | F | G | T | C | T | V | Q | R | L | A | H | Q | I | Y | Q | F | T |
| 125 | G | C | R | F | G | T | C | T | V | Q | R | L | A | H | Q | I | Y | Q | F | T |
| 126 | K | C | N | T | A | T | C | T | V | Q | R | L | A | E | Q | I | Y | Q | F | T |
| 127 | G | C | R | F | G | T | C | T | V | Q | R | L | A | H | Q | I | Y | Q | F | T |
| 128 | K | C | N | T | A | T | C | A | T | Q | R | L | A | N | F | L | V | H | S | S |
| 129 | G | C | R | T | G | T | C | T | V | Q | R | L | A | H | Q | I | Y | Q | F | T |
| 130 | K | C | N | T | A | T | C | T | V | D | R | L | A | H | Q | I | A | Q | F | T |
| 131 | G | C | R | F | G | T | C | T | V | Q | R | L | A | H | Q | I | Y | O | F | T |
| 132 | G | C | R | F | G | T | C | T | V | Q | R | L | A | H | Q | I | Y | Q | F | T |
| 133 | K | C | N | T | A | T | C | T | V | A | R | L | A | E | Q | I | Y | Q | F | T |
| 134 | G | C | R | F | G | T | C | T | V | Q | R | L | A | H | Q | I | Y | Q | F | T |
| 135 | G | C | R | F | G | T | C | T | V | Q | R | L | A | H | Q | I | Y | Q | F | T |
| 136 | K | C | N | T | A | T | C | T | V | Q | R | L | A | E | Q | I | K | Q | F | T |
| 137 | K | C | N | T | A | T | C | T | V | A | R | L | A | H | Q | I | N | Q | F | T |
| 138 | G | C | R | T | G | T | C | T | V | Q | R | L | A | H | Q | I | Y | Q | F | T |
| 139 | G | C | R | T | G | T | C | T | V | Q | R | L | A | H | Q | I | Y | Q | F | T |
| 140 | G | C | R | F | G | T | C | T | V | Q | R | L | A | H | Q | I | Y | Q | F | T |
| 141 | G | C | R | F | G | T | C | T | V | Q | R | L | A | H | Q | I | Y | Q | F | T |
| 142 | G | C | R | T | G | T | C | T | V | Q | R | L | A | H | Q | I | Y | Q | F | T |
| 143 | G | C | R | T | G | T | C | T | V | Q | R | L | A | H | Q | I | Y | Q | F | T |
| 144 | K | C | N | T | A | T | C | T | V | Q | R | L | A | E | Q | I | A | Q | F | T |
| 145 | G | C | R | F | G | T | C | T | V | Q | R | L | A | H | Q | I | Y | Q | F | T |
| 146 | G | C | R | F | G | T | C | T | V | Q | R | L | A | H | Q | I | Y | Q | F | T |
| 147 | G | C | R | I | G | T | C | T | V | Q | R | L | A | H | Q | I | Y | Q | F | T |
| 148 | K | C | N | T | A | T | C | A | T | Q | R | L | A | N | F | L | V | H | S | S |
| 149 | K | C | N | T | A | T | C | A | T | Q | R | L | A | N | F | L | V | H | S | S |
| 150 | G | C | R | W | G | T | C | T | V | Q | R | L | A | H | Q | I | Y | Q | F | T |
| 151 | G | C | R | T | G | T | C | T | V | Q | R | L | A | H | Q | I | Y | Q | F | T |
| 152 | G | C | R | F | G | T | C | T | V | Q | R | L | A | H | Q | I | Y | Q | F | T |
| 153 | K | C | N | T | A | T | C | T | V | Q | Cit | L | A | E | Q | I | A | Q | F | T |
| 154 | G | C | R | F | G | T | C | T | V | Q | R | L | A | H | Q | I | Y | Q | F | T |
| 155 | G | C | R | F | G | T | C | T | V | Q | R | L | A | H | Q | I | Y | Q | F | T |
| 156 | G | C | R | T | G | T | C | T | V | Q | R | L | A | H | Q | I | Y | Q | F | T |
| 157 | G | C | R | T | G | T | C | T | V | Q | R | L | A | H | Q | I | Y | Q | F | T |
| 158 | G | C | R | F | G | T | C | T | V | Q | R | L | A | H | Q | I | Y | Q | F | T |
| 159 | K | C | N | T | A | T | C | T | V | I | R | L | A | H | Q | I | Y | Q | F | T |
| 160 | G | C | R | T | G | T | C | T | V | Q | R | L | A | H | Q | I | Y | Q | I | T |
| 161 | G | C | R | T | G | T | C | T | V | Q | R | L | A | H | Q | I | Y | Q | F | T |
| 162 | K | C | N | T | A | T | C | A | T | Q | R | L | A | N | F | L | V | H | S | S |
| 163 | G | C | R | T | G | T | C | T | V | Q | R | L | A | H | Q | I | Y | Q | F | T |
| 164 | G | C | R | F | G | T | C | T | V | Q | R | L | A | H | Q | I | Y | Q | F | T |
| 165 | G | C | R | T | G | T | C | T | V | Q | R | L | A | H | Q | I | Y | Q | F | T |
| 166 | G | C | R | T | G | T | C | T | V | Q | R | L | A | H | Q | I | Y | Q | F | T |
| 167 | G | C | R | F | G | T | C | T | V | Q | R | L | A | H | Q | I | Y | Q | F | T |
| 168 | G | C | R | F | G | T | C | T | V | Q | R | L | A | H | Q | I | Y | Q | F | T |
| 169 | G | C | R | F | G | T | C | T | V | Q | R | L | A | H | Q | I | Y | Q | F | T |
| 170 | G | C | R | T | G | T | C | T | V | Q | R | L | A | H | Q | I | Y | Q | F | T |
| 171 | G | C | R | W | G | T | C | T | V | Q | R | L | A | H | Q | I | Y | Q | F | T |
| 172 | G | C | R | T | G | T | C | T | V | Q | R | L | A | H | Q | I | Y | Q | F | T |
| 173 | G | C | R | M | G | T | C | T | V | Q | R | L | A | H | Q | I | Y | Q | F | T |
| 174 | G | C | R | F | G | T | C | T | V | Q | R | L | A | H | Q | I | Y | Q | F | T |
| 175 | G | C | R | F | G | T | C | T | V | Q | R | L | A | H | Q | I | Y | Q | F | T |
| 176 | G | C | R | T | G | T | C | T | V | Q | R | L | A | H | Q | I | Y | Q | F | T |
| 177 | G | C | R | T | G | T | C | T | V | Q | R | L | A | H | Q | I | Y | Q | F | T |
| 178 | G | C | R | T | G | T | C | T | V | Q | R | L | A | H | Q | I | Y | Q | F | T |
| 179 | G | C | R | T | G | T | C | T | V | Q | R | L | A | H | Q | I | Y | Q | F | T |
| 180 | K | C | N | T | A | T | C | A | T | Q | R | L | A | N | F | L | V | H | S | S |
| 181 | K | C | N | T | A | T | C | T | V | Q | R | L | A | H | Q | I | Y | — | F | T |
| 182 | G | C | R | F | G | T | C | T | V | Q | R | L | A | H | Q | I | Y | Q | F | T |
| 183 | G | C | R | T | G | T | C | T | V | Q | R | L | A | H | Q | I | Y | Q | F | T |
| 184 | G | C | R | T | G | T | C | T | V | Q | R | L | A | H | Q | I | Y | Q | F | T |
| 185 | G | C | R | T | G | T | C | T | V | Q | R | L | A | H | Q | I | Y | Q | F | T |
| 186 | G | C | R | T | G | T | C | T | V | Q | R | L | A | H | Q | I | Y | Q | F | T |
| 187 | G | C | R | T | G | T | C | T | V | Q | R | L | A | H | Q | I | Y | Q | F | T |
| 188 | G | C | R | T | G | T | C | T | V | Q | R | L | A | H | Q | I | Y | Q | F | T |
| 189 | G | C | R | T | G | T | C | T | V | Q | R | L | A | H | Q | I | Y | Q | F | T |
| 190 | G | C | R | T | G | T | C | T | V | Q | R | L | A | H | Q | I | Y | Q | F | T |
| 191 | G | C | R | T | G | T | C | T | V | Q | R | L | A | H | Q | I | Y | Q | F | T |
| 192 | G | C | R | M | G | T | C | T | V | Q | R | L | A | H | Q | I | Y | Q | F | T |
| 193 | G | C | R | T | G | T | C | T | V | Q | R | L | A | H | Q | I | H | Q | F | T |
| 194 | K | C | R | M | G | T | C | T | V | Q | R | L | A | H | Q | I | Y | Q | F | T |
| 195 | K | C | N | T | A | T | C | T | V | Q | R | L | A | H | Q | I | Y | — | Q | T |
| 196 | G | C | R | T | G | T | C | T | V | Q | R | L | A | H | Q | I | A | Q | F | T |
| 197 | G | C | R | T | G | T | C | T | V | Q | R | L | A | H | Q | I | Y | Q | F | T |
| 198 | G | C | R | I | G | T | C | T | V | Q | R | L | A | H | Q | I | Y | Q | F | T |
| 199 | G | C | R | T | G | T | C | T | V | Q | R | L | A | H | Q | I | Y | Q | F | T |
| 200 | G | C | R | F | G | T | C | T | V | Q | R | L | A | H | F | L | Y | Q | F | T |
| 201 | G | C | R | T | G | T | C | T | V | Q | R | L | A | H | Q | I | Y | Q | F | T |
| 202 | G | C | R | W | G | T | C | T | V | Q | R | L | A | H | Q | I | Y | Q | F | T |
| 203 | G | C | R | T | G | T | C | T | V | Q | R | L | A | H | Q | I | Y | Q | F | T |
| 204 | G | C | R | T | G | T | C | T | V | Q | R | L | A | H | Q | I | Y | Q | F | T |
| 205 | K | C | R | F | G | T | C | T | V | Q | R | L | A | H | Q | I | Y | Q | F | T |
| 206 | G | C | R | T | G | T | C | T | V | Q | R | L | A | H | Q | I | Y | Q | F | T |
| 207 | K | C | N | T | A | T | C | A | T | Q | R | L | A | N | F | L | V | H | S | S |
| 208 | G | C | R | F | G | T | C | T | V | Q | R | L | A | H | F | L | Y | Q | F | T |
| 209 | G | C | R | T | G | T | C | T | V | Q | R | L | A | H | Q | I | Y | Q | F | T |
| 210 | K | C | N | T | A | T | C | T | V | Q | R | L | A | H | Q | A | Y | Q | F | T |
| 211 | G | C | R | T | G | T | C | T | V | Q | R | L | A | H | Q | I | Y | Q | F | T |
| 212 | G | C | R | F | G | T | C | T | V | Q | R | L | A | H | Q | I | Y | Q | F | T |
| 213 | G | C | R | T | G | T | C | T | V | Q | R | L | A | H | Q | I | W | O | F | T |
| 214 | G | C | R | T | G | T | C | T | V | Q | R | L | A | H | Q | I | Y | Q | F | T |
| 215 | G | C | R | T | G | T | C | T | V | Q | R | L | A | H | Q | I | Y | Q | F | T |
| 216 | G | C | R | T | G | T | C | T | V | Q | R | L | A | H | Q | I | Y | Q | F | T |
| 217 | G | C | R | F | G | T | C | T | V | Q | R | L | A | H | Q | L | Y | Q | F | T |
| 218 | G | C | R | T | G | T | C | T | V | Q | R | L | A | H | Q | I | Y | Q | F | T |
| 219 | G | C | R | T | G | T | C | T | V | Q | R | L | A | H | Q | I | Y | Q | F | T |
| 220 | G | C | R | T | G | T | C | T | V | Q | R | L | A | H | Q | I | Y | Q | F | T |
| 221 | K | C | N | T | A | T | C | T | V | Q | R | L | A | E | Q | I | Y | Q | F | T |
| 222 | G | C | R | M | G | T | C | T | V | Q | R | L | A | H | Q | I | Y | Q | F | T |
| 223 | G | C | R | F | G | T | C | T | V | Q | R | L | A | H | F | L | Y | Q | F | T |
| 224 | G | C | R | T | G | T | C | T | V | O | R | L | A | H | Q | I | Y | Q | F | T |
| 225 | K | C | Z | T | L | T | C | T | V | Q | R | L | A | H | Q | I | Y | Q | F | T |
| 226 | G | C | R | T | G | T | C | T | V | Q | R | L | A | H | Q | I | Y | Q | F | T |
| 227 | G | C | R | T | G | T | C | T | V | Q | R | L | A | H | Q | I | H | Q | F | T |
| 228 | G | C | R | T | G | T | C | T | V | Q | R | L | A | H | Q | I | H | Q | F | T |
| 229 | G | C | R | F | G | T | C | T | V | Q | R | L | A | H | Q | I | Y | Q | F | T |
| 230 | G | C | R | T | G | T | C | T | V | Q | R | L | A | H | Q | I | Y | Q | F | T |
| 231 | K | C | N | T | Y | T | C | T | V | Q | R | L | A | H | Q | I | Y | Q | F | T |
| 232 | G | C | R | T | G | T | C | T | V | Q | R | L | A | H | Q | I | Y | Q | F | T |
| 233 | K | C | N | T | A | T | C | T | V | Q | R | L | A | E | Q | I | Y | Q | F | T |
| 234 | K | C | N | T | A | T | C | A | T | Q | R | L | A | N | F | L | V | Q | F | T |
| 235 | G | C | R | T | G | T | C | T | V | Q | R | L | A | H | Q | I | Y | Q | F | T |
| 236 | G | C | R | T | G | T | C | T | V | Q | R | L | A | H | Q | I | Y | Q | F | T |
| 237 | G | C | R | T | G | T | C | T | V | Q | R | L | A | H | Q | I | Y | Q | F | T |
| 238 | G | C | R | T | G | T | C | T | V | Q | R | L | A | H | Q | I | Y | Q | F | T |
| 239 | G | C | R | T | G | T | C | T | V | Q | R | L | A | H | Q | I | Y | Q | F | T |
| 240 | G | C | R | F | G | T | C | T | V | Q | R | L | A | H | Q | I | Y | Q | F | T |
| 241 | G | C | R | T | G | T | C | T | V | O | R | L | A | H | Q | I | Y | Q | F | T |
| 242 | G | C | R | T | G | T | C | T | V | Q | R | L | A | H | Q | I | Y | Q | F | T |
| 243 | K | C | N | T | A | T | C | T | V | Q | Cit | L | A | A | Q | I | Y | Q | F | T |
| 244 | K | C | N | T | S | T | C | T | V | A | Cit | L | A | D | Q | I | T | Q | F | S |
| 245 | R | C | N | A | S | T | C | T | V | N | Cit | L | A | D | Q | I | T | Q | F | S |
| 246 | K | C | N | T | A | T | C | T | V | Q | Cit | L | A | H | Q | I | A | Q | F | T |
| 247 | K | C | N | T | S | T | C | T | V | A | Cit | L | A | D | Q | I | T | Q | F | S |
| 248 | K | C | N | T | A | T | C | T | V | Q | Cit | L | A | H | Q | I | Y | Q | F | T |
| 249 | K | C | N | T | A | T | C | T | V | Q | Cit | L | A | H | Q | I | Y | Q | F | A |
| 250 | K | C | N | T | A | T | C | T | V | Q | Cit | L | A | H | Q | I | Y | Q | F | T |
| 251 | K | C | N | T | A | T | C | T | V | Q | Cit | L | A | H | Q | I | Y | Q | F | T |
| 252 | K | C | N | T | A | T | C | T | V | Q | Cit | L | A | H | Q | I | Y | Q | F | T |
| 253 | K | C | N | T | A | T | C | T | V | Q | Cit | L | A | H | Q | I | Y | Q | F | — |
| 254 | K | C | N | T | A | T | C | T | V | Q | Cit | L | A | H | Q | I | Y | Q | F | T |
| 255 | K | C | N | T | A | T | C | T | V | Q | Cit | L | A | H | Q | I | Y | Q | F | T |
| 256 | R | C | Q | T | S | T | C | T | V | A | Cit | L | A | E | Q | I | A | Q | Y | T |
| 257 | K | C | N | T | A | T | C | T | V | Q | Cit | L | A | H | Q | I | Y | Q | F | T |
| 258 | K | C | N | T | A | T | C | T | V | Q | Cit | L | A | H | Q | I | Y | Q | F | T |
| 259 | G | C | R | F | G | T | C | T | V | Q | Cit | L | A | H | Q | I | Y | Q | F | T |
| 260 | G | C | R | F | G | T | C | T | V | Q | Cit | L | A | H | Q | I | Y | Q | F | T |
| 261 | G | C | R | T | G | T | C | T | V | Q | Cit | L | A | H | Q | I | Y | Q | F | T |
| 262 | K | C | N | T | A | T | C | T | V | O | Cit | L | A | H | Q | I | Y | Q | F | T |
| 263 | G | C | R | F | G | T | C | T | V | Q | Cit | L | A | H | F | L | Y | Q | F | T |
| 264 | G | C | R | F | G | T | C | T | V | Q | Cit | L | A | H | F | L | Y | Q | F | T |
| 265 | K | C | N | T | A | T | C | T | V | Q | Cit | L | A | H | Q | I | Y | Q | F | — |
| 266 | K | C | N | T | A | T | C | T | V | Q | Cit | L | A | E | Q | I | Y | Q | F | T |
| 267 | G | C | R | F | G | T | C | T | V | Q | Cit | L | A | H | Q | I | Y | Q | F | T |
| 268 | K | C | N | T | A | T | C | T | V | Q | Cit | L | A | H | Q | I | Y | Q | F | T |
| 269 | G | C | R | T | G | T | C | T | V | Q | Cit | L | A | H | Q | I | Y | Q | F | T |
| 270 | G | C | R | F | G | T | C | T | V | Q | Cit | L | A | H | Q | I | Y | Q | F | T |
| 271 | G | C | R | F | G | T | C | T | V | Q | Cit | L | A | H | Q | I | Y | Q | F | T |
| 272 | K | C | N | T | A | T | C | T | V | Q | Cit | L | A | H | Q | I | Y | Q | F | T |
| 273 | G | C | R | T | G | T | C | T | V | Q | Cit | L | A | H | Q | I | Y | Q | F | T |
| 274 | G | C | R | F | G | T | C | T | V | Q | Cit | L | A | H | Q | I | Y | Q | F | T |
| 275 | G | C | R | F | G | T | C | T | V | Q | Cit | L | A | H | Q | I | Y | Q | F | T |
| 276 | K | C | N | T | A | T | C | T | V | Q | Cit | L | A | H | Q | I | Y | Q | A | T |
| 277 | G | C | R | T | G | T | C | T | V | Q | Cit | L | A | H | Q | I | Y | Q | F | T |
| 278 | G | C | R | F | G | T | C | T | V | Q | Cit | L | A | H | Q | I | Y | Q | F | T |
| 279 | G | C | R | F | G | T | C | T | V | Q | Cit | L | A | H | F | L | Y | Q | F | T |
| 280 | G | C | R | T | G | T | C | T | V | Q | Cit | L | A | H | Q | I | Y | Q | F | T |
| 281 | G | C | R | T | G | T | C | T | V | Q | Cit | L | A | H | Q | I | Y | Q | F | T |
| 282 | G | C | R | F | G | T | C | T | V | Q | Cit | L | A | H | F | L | Y | Q | F | T |
| 283 | G | C | R | F | G | T | C | T | V | Q | Cit | L | A | H | Q | I | Y | Q | F | T |
| 284 | G | C | R | F | G | T | C | T | V | Q | Cit | L | A | H | Q | I | Y | Q | F | T |
| 285 | G | C | R | T | G | T | C | T | V | Q | Cit | L | A | H | Q | I | Y | Q | F | T |
| 286 | G | C | R | F | G | T | C | T | V | Q | Cit | L | A | H | Q | I | Y | Q | F | T |
| 287 | G | C | R | F | G | T | C | T | V | Q | Cit | L | A | H | Q | I | Y | Q | F | T |
| 288 | G | C | R | F | G | T | C | T | V | Q | Cit | L | A | H | Q | I | Y | Q | F | T |
| 289 | G | C | R | F | G | T | C | T | V | Q | Cit | L | A | H | Q | I | Y | Q | F | T |
| 290 | G | C | R | T | G | T | C | T | V | Q | Cit | L | A | H | Q | I | Y | Q | F | T |
| 291 | G | C | R | F | G | T | C | T | V | Q | Cit | L | A | H | Q | I | Y | Q | F | T |
| 292 | G | C | R | T | G | T | C | T | V | Q | Cit | L | A | H | Q | I | Y | Q | F | T |
| 293 | G | C | R | F | G | T | C | T | V | Q | Cit | L | A | H | Q | I | Y | Q | F | T |
| 294 | G | C | R | F | G | T | C | T | V | Q | Cit | L | A | H | Q | I | Y | Q | F | T |
| 295 | G | C | R | I | G | T | C | T | V | Q | Cit | L | A | H | Q | I | Y | Q | F | T |
| 296 | G | C | R | W | G | T | C | T | V | Q | Cit | L | A | H | Q | I | Y | Q | F | T |
| 297 | G | C | R | T | G | T | C | T | V | Q | Cit | L | A | H | Q | I | Y | Q | F | T |
| 298 | G | C | R | T | G | T | C | T | V | Q | Cit | L | A | H | Q | I | Y | Q | F | T |
| 299 | G | C | R | T | G | T | C | T | V | Q | Cit | L | A | H | Q | I | Y | Q | F | T |
| 300 | G | C | R | F | G | T | C | T | V | Q | Cit | L | A | H | Q | I | Y | Q | F | T |
| 301 | G | C | R | T | G | T | C | T | V | Q | Cit | L | A | H | Q | I | Y | Q | F | T |
| 302 | G | C | R | T | G | T | C | T | V | Q | Cit | L | A | H | Q | I | Y | Q | F | T |
| 303 | G | C | R | T | G | T | C | T | V | Q | Cit | L | A | H | Q | I | Y | Q | F | T |
| 304 | G | C | R | T | G | T | C | T | V | Q | Cit | L | A | H | Q | I | Y | Q | F | T |
| 305 | G | C | R | F | G | T | C | T | V | Q | Cit | L | A | H | Q | I | Y | Q | F | T |
| 306 | K | C | N | T | S | T | C | T | V | A | W | L | A | D | Q | I | T | Q | F | S |
| 307 | R | C | N | A | S | T | C | T | V | N | W | L | A | D | Q | I | T | Q | F | S |
| 308 | K | C | N | T | A | T | C | T | V | Q | W | L | A | H | A | I | Y | Q | F | T |
| 309 | K | C | N | T | A | T | C | T | V | Q | W | L | A | H | Q | I | A | Q | F | T |
| 310 | K | C | N | T | S | T | C | T | V | A | W | L | A | D | Q | I | T | Q | F | S |
| 311 | G | C | R | F | G | T | C | T | V | Q | W | L | A | N | F | L | Y | Q | F | T |
| 312 | K | C | N | T | A | T | C | T | V | Q | W | L | A | H | Q | I | Y | Q | F | T |
| 313 | K | C | N | T | A | T | C | T | V | Q | W | L | A | H | Q | I | Y | Q | F | A |
| 314 | K | C | N | T | A | T | C | T | V | Q | W | L | A | H | Q | I | Y | Q | F | T |
| 315 | K | C | N | T | A | T | C | T | V | Q | W | L | A | H | Q | I | Y | Q | F | T |
| 316 | K | C | N | T | A | T | C | T | V | Q | W | L | A | H | Q | I | Y | Q | F | T |
| 317 | K | C | N | T | A | T | C | T | V | Q | W | L | A | H | Q | I | Y | Q | F | — |
| 318 | K | C | N | T | A | T | C | T | V | Q | W | L | A | H | Q | I | Y | Q | F | T |
| 319 | K | C | N | T | A | T | C | T | V | Q | W | L | A | H | Q | I | Y | Q | F | T |
| 320 | K | C | N | T | A | T | C | T | V | Q | W | L | A | H | Q | I | Y | Q | F | T |
| 321 | K | C | N | T | A | T | C | T | V | Q | W | L | A | E | Q | I | Y | Q | F | T |
| 322 | G | C | R | F | G | T | C | T | V | Q | W | L | A | H | Q | I | Y | Q | F | T |
| 323 | G | C | R | T | G | T | C | T | V | Q | W | L | A | H | Q | I | Y | Q | F | T |
| 324 | K | C | N | T | A | T | C | T | V | Q | W | L | A | H | Q | I | Y | Q | F | T |
| 325 | K | C | N | T | A | T | C | T | V | Q | W | L | A | H | Q | I | Y | Q | F | T |
| 326 | G | C | R | F | G | T | C | T | V | Q | W | L | A | H | F | L | Y | Q | F | T |
| 327 | G | C | R | F | G | T | C | T | V | Q | W | L | A | H | F | L | Y | Q | F | T |
| 328 | K | C | N | T | A | T | C | T | V | Q | W | L | A | H | Q | I | Y | Q | F | T |
| 329 | K | C | N | T | A | T | C | T | V | Q | W | L | A | H | Q | I | Y | Q | F | — |
| 330 | K | C | N | T | A | T | C | T | V | Q | W | L | A | H | Q | I | Y | Q | F | T |
| 331 | G | C | R | F | G | T | C | T | V | Q | W | L | A | H | Q | I | Y | Q | F | T |
| 332 | G | C | R | T | G | T | C | T | V | Q | W | L | A | H | Q | I | Y | Q | F | T |
| 333 | K | C | N | A | A | T | C | T | V | Q | W | L | A | E | Q | I | Y | Q | F | T |
| 334 | K | C | N | T | A | T | C | T | V | Q | W | L | A | H | Q | I | Y | Q | F | T |
| 335 | G | C | R | T | G | T | C | T | V | Q | W | L | A | H | Q | I | Y | Q | F | T |
| 336 | G | C | R | F | G | T | C | T | V | Q | W | L | A | H | Q | I | Y | Q | F | T |
| 337 | G | C | R | F | G | T | C | T | V | Q | W | L | A | H | Q | I | Y | Q | F | T |
| 338 | K | C | N | T | A | T | C | T | V | Q | W | L | A | H | Q | I | Y | Q | A | T |
| 339 | G | C | R | T | G | T | C | T | V | Q | W | L | A | H | Q | I | Y | Q | F | T |
| 340 | G | C | R | F | G | T | C | T | V | Q | W | L | A | H | Q | I | Y | Q | F | T |
| 341 | G | C | R | T | G | T | C | T | V | Q | W | L | A | H | Q | I | Y | Q | F | T |
| 342 | G | C | R | T | G | T | C | T | V | Q | W | L | A | H | Q | I | Y | Q | F | T |
| 343 | G | C | R | F | G | T | C | T | V | Q | W | L | A | H | Q | I | Y | Q | F | T |
| 344 | G | C | R | T | G | T | C | T | V | Q | W | L | A | H | Q | I | Y | Q | F | T |
| 345 | G | C | R | F | G | T | C | T | V | Q | W | L | A | H | Q | I | Y | Q | F | T |
| 346 | G | C | R | T | G | T | C | T | V | Q | W | L | A | H | Q | I | Y | Q | F | T |
| 347 | G | C | R | T | G | T | C | T | V | Q | W | L | A | H | Q | I | Y | Q | F | T |
| 348 | G | C | R | T | G | T | C | T | V | Q | W | L | A | H | Q | I | Y | Q | F | T |
| 349 | G | C | R | T | G | T | C | T | V | Q | W | L | A | H | Q | I | Y | Q | F | T |
| 350 | G | C | R | F | G | T | C | T | V | Q | W | L | A | H | Q | I | Y | Q | F | T |
| 351 | G | C | R | I | G | T | C | T | V | Q | W | L | A | H | Q | I | Y | Q | F | T |
| 352 | G | C | R | T | G | T | C | T | V | Q | W | L | A | H | Q | I | Y | Q | F | T |
| 353 | G | C | R | F | G | T | C | T | V | Q | W | L | A | H | Q | I | Y | Q | F | T |
| 354 | G | C | R | T | G | T | C | T | V | Q | W | L | A | H | Q | I | Y | Q | F | T |
| 355 | G | C | R | T | G | T | C | T | V | Q | W | L | A | H | Q | I | Y | Q | F | T |
| 356 | G | C | R | T | G | T | C | T | V | Q | W | L | A | H | Q | I | Y | Q | F | T |
| 357 | G | C | R | T | G | T | C | T | V | Q | W | L | A | H | Q | I | Y | Q | F | T |
| 358 | G | C | R | T | G | T | C | T | V | Q | W | L | A | H | Q | I | Y | Q | F | T |
| 359 | G | C | R | T | G | T | C | T | V | Q | W | L | A | H | Q | I | Y | Q | F | T |
| 360 | G | C | R | T | G | T | C | T | V | Q | W | L | A | H | Q | I | Y | Q | F | T |
| 361 | K | C | N | F | A | T | C | T | V | Q | R | L | A | H | Q | I | Y | Q | F | T |
| 362 | G | C | R | F | G | T | C | T | V | Q | R | L | A | H | Q | I | Y | Q | F | T |
| 363 | K | C | N | F | A | T | C | A | T | Q | R | L | A | N | F | L | V | Q | F | T |
| 364 | G | C | R | F | G | T | C | T | V | Q | R | L | A | H | F | L | Y | Q | F | T |
| 365 | G | C | R | F | G | T | C | T | V | Q | R | L | A | H | F | L | Y | Q | F | T |
| 366 | G | C | R | F | G | T | C | T | V | Q | R | L | A | H | Q | I | Y | Q | F | T |
| 367 | G | C | R | F | G | T | C | T | V | Q | R | L | A | H | Q | I | Y | Q | F | T |
| 368 | G | C | R | F | G | T | C | T | V | Q | R | L | A | H | Q | I | Y | Q | F | T |
| 369 | G | C | R | F | G | T | C | T | V | Q | R | L | A | H | F | L | Y | Q | F | T |
| 370 | K | C | N | F | A | T | C | T | V | Q | R | L | A | H | Q | I | Y | Q | F | T |
| 371 | G | C | R | F | G | T | C | T | V | Q | R | L | A | H | Q | I | Y | Q | F | T |
| 372 | G | C | R | F | G | T | C | T | V | Q | R | L | A | N | Q | I | Y | Q | F | T |
| 373 | G | C | R | F | G | T | C | T | V | Q | R | L | A | H | Q | I | Y | Q | F | T |
| 374 | G | C | R | F | G | T | C | T | V | Q | R | L | A | H | Q | I | Y | Q | F | T |
| 375 | G | C | R | F | G | T | C | T | V | Q | R | L | A | H | Q | I | Y | Q | F | T |
| 376 | G | C | R | F | G | T | C | T | V | Q | R | L | A | H | F | L | Y | Q | F | T |
| 377 | G | C | R | F | A | T | C | T | V | Q | R | L | A | H | F | L | Y | Q | F | T |
| 378 | K | C | N | F | A | T | C | T | V | Q | R | L | A | H | Q | I | Y | Q | F | T |
| 379 | G | C | R | F | G | T | C | T | V | Q | R | L | A | H | Q | I | Y | Q | F | T |
| 380 | G | C | R | F | A | T | C | T | V | Q | R | L | A | H | Q | I | Y | Q | F | T |
| 381 | G | C | R | F | G | T | C | T | V | Q | R | L | A | H | Q | I | Y | Q | F | T |
| 382 | G | C | R | F | G | T | C | T | V | Q | R | L | A | H | Q | I | Y | Q | F | T |
| 383 | G | C | R | F | G | T | C | T | V | Q | R | L | A | H | Q | I | Y | Q | F | T |
| 384 | G | C | R | F | G | T | C | T | V | Q | R | L | A | H | Q | I | Y | Q | F | T |
| 385 | G | C | R | F | G | T | C | T | V | Q | R | L | A | H | Q | I | Y | Q | F | T |
| 386 | G | C | R | F | G | T | C | T | V | Q | R | L | A | N | F | L | Y | Q | F | T |
| 387 | G | C | R | F | G | T | C | T | V | Q | R | L | A | H | Q | I | Y | Q | F | T |
| 388 | G | C | R | F | G | T | C | T | V | Q | R | L | A | H | F | L | Y | Q | F | T |
| 389 | K | C | N | F | A | T | C | A | T | Q | R | L | A | N | F | L | V | Q | F | T |
| 390 | G | C | R | F | G | T | C | T | V | Q | R | L | A | H | F | L | Y | Q | F | T |
| 391 | G | C | R | F | G | T | C | T | V | Q | R | L | A | H | Q | I | Y | Q | F | T |
| 392 | G | C | R | F | G | T | C | T | V | Q | R | L | A | H | F | L | Y | Q | F | T |

| ID | 21 | 22 | 23 | 24 | 25 | 26 | 27 | 28 | 29 | 30 | 31 | 32 | 33 | 34 | 35 | 36 | 37 | 38 |
|---|---|---|---|---|---|---|---|---|---|---|---|---|---|---|---|---|---|---|
| 1 | D | K | D | K | D | N | V | A | P | R | S | K | I | S | P | Q | G | Y |
| 2 | N | N | F | — | G | A | I | L | S | S | T | N | V | G | S | N | T | Y |
| 3 | D | K | D | K | D | N | V | A | P | R | T | K | V | G | S | N | G | Hyp |
| 4 | D | K | D | K | D | N | V | A | P | P | T | N | V | G | S | N | G | Hyp |
| 5 | D | K | D | K | D | N | V | A | P | P | T | N | V | G | S | N | G | Hyp |
| 6 | D | K | D | K | D | N | V | A | P | P | T | N | V | G | S | K | G | Hyp |
| 7 | D | K | D | K | D | N | V | A | P | P | T | N | V | G | S | N | G | Hyp |
| 8 | N | K | D | K | A | Q | V | S | P | P | T | E | V | G | P | N | S | Hyp |
| 9 | N | K | D | K | A | Q | V | S | P | P | T | E | V | G | P | N | S | Hyp |
| 10 | D | K | D | K | D | N | V | A | P | P | T | E | V | G | S | N | G | Hyp |
| 11 | D | K | D | K | D | N | V | A | P | P | T | N | V | G | S | N | G | Hyp |
| 12 | D | K | D | K | D | N | K | A | P | P | T | N | V | G | S | N | G | Hyp |
| 13 | D | K | D | K | D | N | V | A | P | P | T | N | V | G | S | N | G | Hyp |
| 14 | D | K | D | K | D | N | V | A | P | P | T | N | V | G | S | N | G | Hyp |
| 15 | D | K | D | K | A | N | V | S | P | P | T | E | V | G | P | N | S | Hyp |
| 16 | D | K | D | K | D | N | V | A | P | R | T | N | V | G | P | Q | G | Y |
| 17 | D | K | D | K | D | N | V | A | P | R | T | E | V | G | S | N | G | Hyp |
| 18 | D | K | D | K | D | N | V | A | P | P | T | E | V | G | S | N | G | P |
| 19 | N | N | F | G | A | — | I | L | P | R | T | K | V | G | S | N | G | Y |
| 20 | D | K | D | K | D | N | V | A | P | R | T | K | V | G | S | N | G | Hyp |
| 21 | D | K | D | K | D | N | V | A | P | P | T | N | V | G | S | N | G | Hyp |
| 22 | D | K | D | K | D | N | V | A | P | P | T | N | V | G | S | N | G | Hyp |
| 23 | D | K | D | K | D | N | V | A | P | P | T | N | V | G | S | N | G | Hyp |
| 24 | A | K | D | K | D | N | V | A | P | P | T | N | V | G | S | N | G | Hyp |
| 25 | D | K | D | A | D | N | V | A | P | P | T | N | V | G | S | N | G | Hyp |
| 26 | D | K | D | K | A | N | V | A | P | P | T | N | V | G | S | N | G | Hyp |
| 27 | D | K | D | K | D | N | A | A | P | P | T | N | V | G | S | N | G | Hyp |
| 28 | D | K | D | K | D | N | V | A | P | A | T | N | V | G | S | N | G | Hyp |
| 29 | T | K | D | K | D | N | V | A | P | P | T | N | V | G | S | N | G | Hyp |
| 30 | D | K | D | K | D | N | V | — | P | P | T | N | V | G | S | N | G | Hyp |
| 31 | D | K | D | K | D | N | V | — | A | P | T | N | V | G | S | N | G | Hyp |
| 32 | D | K | D | K | D | Q | V | A | P | P | T | N | V | G | S | N | S | Hyp |
| 33 | D | K | D | K | D | Q | V | A | P | P | T | E | V | G | P | N | S | Hyp |
| 34 | D | K | D | K | D | N | V | A | P | R | T | K | V | G | S | N | G | Y |
| 35 | D | K | D | K | D | N | V | A | P | P | T | N | V | G | S | N | G | Hyp |
| 36 | D | K | D | K | D | N | V | A | P | P | T | N | V | G | S | N | G | Hyp |
| 37 | D | K | D | K | D | N | V | A | P | P | T | N | V | G | S | N | G | Hyp |
| 38 | D | K | D | K | D | N | V | A | P | P | T | N | V | G | S | N | G | Hyp |
| 39 | D | K | D | K | D | N | V | A | P | P | T | N | V | G | S | N | G | Hyp |
| 40 | D | K | D | K | K | N | V | A | P | P | T | N | V | G | S | N | G | Hyp |
| 41 | D | K | D | K | D | K | V | A | P | P | T | N | V | G | S | N | G | Hyp |
| 42 | D | K | D | K | D | N | V | A | P | P | T | N | V | G | S | N | G | Hyp |
| 43 | D | K | A | K | D | N | V | A | P | P | T | N | V | G | S | N | G | Hyp |
| 44 | D | K | D | K | D | N | V | A | P | P | Y | N | E | G | S | N | G | Hyp |
| 45 | D | K | D | K | D | N | V | A | P | R | T | K | V | G | P | Q | G | Hyp |
| 46 | D | K | D | K | D | N | V | A | P | R | T | N | I | G | P | Q | G | Hyp |
| 47 | D | K | D | K | D | N | V | A | P | P | T | N | V | G | S | N | G | Hyp |
| 48 | D | K | D | K | D | N | V | A | P | P | T | N | V | G | S | N | G | Hyp |
| 49 | D | K | D | K | D | N | V | A | P | R | T | N | V | G | P | Q | G | Hyp |
| 50 | D | K | D | K | D | N | V | A | P | P | T | N | V | G | S | N | G | Hyp |
| 51 | D | K | D | K | D | N | V | A | P | P | T | N | V | G | S | N | G | Hyp |
| 52 | D | K | D | K | D | A | V | A | P | P | T | N | V | G | S | N | G | Hyp |
| 53 | D | K | D | K | D | N | V | A | A | P | T | N | V | G | S | N | G | Hyp |
| 54 | D | K | D | K | D | N | V | A | P | R | T | N | V | G | P | Q | G | Y |
| 55 | D | K | D | K | D | N | V | A | P | P | T | N | V | G | S | N | G | Hyp |
| 56 | D | K | D | K | D | N | V | A | P | R | T | N | V | G | S | N | T | Y |
| 57 | D | K | D | K | D | N | V | A | P | P | T | N | V | G | S | N | G | Hyp |
| 58 | N | N | F | G | A | — | I | L | S | S | T | K | V | G | S | N | T | Y |
| 59 | N | N | F | G | A | — | I | L | P | S | T | K | V | G | S | N | G | Y |
| 60 | D | K | D | K | D | N | V | A | P | P | T | K | V | G | S | N | G | Hyp |
| 61 | D | K | D | K | D | N | V | A | P | P | T | N | V | G | S | N | G | Hyp |
| 62 | D | K | D | K | D | N | V | A | P | P | T | N | V | G | S | N | G | Hyp |
| 63 | D | K | D | K | D | N | V | A | P | P | T | A | V | G | S | N | G | Hyp |
| 64 | D | A | D | K | D | N | V | A | P | P | T | N | V | G | S | N | G | Hyp |
| 65 | D | K | D | K | D | N | V | A | P | P | T | N | V | G | S | N | G | Hyp |
| 66 | D | K | D | K | D | N | V | S | P | P | T | N | V | G | S | N | G | Hyp |
| 67 | D | K | D | K | D | N | V | A | P | R | T | E | V | G | S | N | G | Hyp |
| 68 | D | K | D | K | D | N | V | A | P | P | T | E | V | G | S | N | G | Hyp |
| 69 | D | K | D | K | D | N | V | A | P | P | T | N | V | G | S | N | S | Hyp |
| 70 | D | K | D | K | D | N | V | A | P | R | T | N | V | G | P | Q | G | Hyp |
| 71 | D | K | D | K | D | N | V | A | P | P | T | N | V | G | S | N | G | Hyp |
| 72 | D | K | D | K | D | N | V | A | P | P | T | N | V | G | S | N | G | Hyp |
| 73 | D | K | D | K | D | N | V | A | P | P | T | Q | V | G | S | N | G | Hyp |
| 74 | D | K | D | K | D | N | V | A | P | P | T | N | V | G | S | N | G | Hyp |
| 75 | D | K | D | K | D | N | V | A | P | P | T | N | V | G | S | N | G | Hyp |
| 76 | — | S | F | (NMe)G | A | — | (NMe)I | L | S | S | T | E | V | G | S | N | T | Hyp |
| 77 | D | K | D | K | D | N | V | A | P | P | T | N | V | G | S | N | G | Hyp |
| 78 | D | K | D | K | D | N | V | A | P | P | T | N | V | G | S | N | G | Hyp |
| 79 | D | K | D | K | D | N | V | A | P | P | T | N | V | G | S | N | G | Hyp |
| 80 | — | S | F | (NMe)G | A | — | (NMe)I | L | S | S | T | E | V | G | S | N | T | Hyp |
| 81 | D | K | D | K | D | N | V | A | P | R | T | N | I | G | P | N | G | Hyp |
| 82 | N | K | D | K | D | N | V | A | P | P | T | N | V | G | S | N | G | Hyp |
| 83 | D | K | D | K | D | N | V | A | P | P | T | N | V | G | S | N | G | Hyp |
| 84 | D | K | D | K | D | N | V | A | P | R | T | N | V | G | P | N | G | Y |
| 85 | D | K | D | K | D | N | V | A | P | P | T | N | V | G | S | N | G | Hyp |
| 86 | D | K | D | K | D | N | V | A | P | P | T | N | V | G | S | N | G | Hyp |
| 87 | D | K | N | K | D | N | V | A | P | P | T | N | V | G | S | N | G | Hyp |
| 88 | D | K | D | K | D | P | V | A | P | P | T | N | V | G | S | N | G | Hyp |
| 89 | D | K | D | K | D | N | V | A | P | P | T | N | V | G | S | N | G | Hyp |
| 90 | D | K | D | K | D | N | V | A | P | R | T | N | V | G | P | N | G | Hyp |
| 91 | D | K | N | K | D | N | V | A | P | P | T | N | V | G | S | N | G | Hyp |
| 92 | D | K | D | K | D | P | V | A | P | P | T | N | V | G | S | N | G | Hyp |
| 93 | N | N | F | G | A | — | I | A | P | S | T | K | V | G | S | N | G | Y |
| 94 | D | K | D | K | D | N | V | A | P | P | T | N | V | G | S | N | G | Hyp |
| 95 | N | K | D | K | D | N | V | A | P | P | T | N | V | G | S | N | G | Hyp |
| 96 | D | K | D | K | D | N | V | A | P | P | T | N | V | G | S | N | T | Hyp |
| 97 | D | K | D | K | D | N | V | A | P | P | T | N | V | G | S | N | G | Hyp |
| 98 | D | K | D | K | D | N | V | A | P | R | T | N | V | S | P | N | G | Y |
| 99 | D | K | D | K | D | N | V | A | P | R | T | K | V | G | S | N | G | Hyp |
| 100 | D | K | D | K | D | Q | V | A | P | P | T | N | V | G | S | N | G | Hyp |
| 101 | D | K | D | K | D | N | V | A | P | P | T | N | V | G | S | N | G | Hyp |
| 102 | D | K | D | K | D | N | V | A | P | R | T | E | V | G | S | N | G | Y |
| 103 | D | K | D | K | D | N | V | A | P | P | T | N | V | G | S | N | G | Hyp |
| 104 | D | K | D | K | D | N | V | A | P | P | T | N | V | G | S | N | G | Hyp |
| 105 | D | K | D | K | D | N | V | A | P | P | T | N | V | G | S | N | G | Hyp |
| 106 | A | K | D | K | D | N | V | A | P | P | T | N | V | G | S | N | G | Hyp |
| 107 | D | K | D | K | D | N | V | A | P | P | T | N | V | G | S | N | G | Hyp |
| 108 | D | K | D | K | A | N | V | A | P | P | T | N | V | G | S | N | G | Hyp |
| 109 | D | K | D | K | D | N | V | A | P | R | T | N | V | G | P | Q | G | Y |
| 110 | D | K | D | K | D | N | V | A | P | P | T | N | V | G | S | N | G | Hyp |
| 111 | D | K | D | K | D | N | V | A | P | R | T | K | I | G | P | Q | G | Hyp |
| 112 | D | K | D | K | D | N | V | A | P | P | T | N | V | G | S | N | G | Hyp |
| 113 | D | K | D | K | D | N | V | A | P | R | T | K | I | S | P | N | G | Hyp |
| 114 | D | K | D | K | D | N | V | A | P | P | T | N | V | G | S | N | G | Hyp |
| 115 | D | K | D | K | D | N | V | A | P | R | T | K | I | G | P | N | G | Hyp |
| 116 | D | K | D | K | D | N | V | A | P | R | T | N | V | S | P | Q | G | Y |
| 117 | D | K | D | K | D | N | V | A | P | R | T | K | V | G | S | N | G | Y |
| 118 | D | K | D | K | D | N | V | A | P | R | T | N | I | G | P | Q | G | Hyp |
| 119 | D | K | D | K | D | N | V | A | P | P | T | N | V | G | S | N | G | Hyp |
| 120 | D | K | D | K | D | P | V | A | P | P | T | N | V | G | S | N | S | Hyp |
| 121 | D | K | D | K | D | N | V | A | P | R | S | N | V | G | P | Q | G | Hyp |
| 122 | D | K | A | K | D | N | V | A | P | P | T | N | V | G | S | N | G | Hyp |
| 123 | D | K | D | G | A | — | I | A | P | R | T | K | I | G | S | Q | G | Y |
| 124 | D | K | D | K | D | N | V | A | P | R | T | K | I | G | P | N | G | Hyp |
| 125 | D | K | D | K | D | N | V | A | P | R | T | N | V | S | P | Q | G | Hyp |
| 126 | D | K | D | K | D | N | V | A | P | P | T | N | V | G | S | N | G | Hyp |
| 127 | D | K | D | K | D | N | V | A | P | R | S | K | I | G | P | O | G | Hyp |
| 128 | N | N | F | G | A | — | I | A | P | S | T | K | I | G | P | N | G | Y |
| 129 | D | K | D | K | D | N | V | A | P | R | S | K | V | G | P | Q | G | Y |
| 130 | D | K | D | K | D | P | V | A | P | P | T | N | V | G | S | N | G | Hyp |
| 131 | D | K | D | K | D | N | V | A | P | R | S | N | V | S | P | Q | G | Y |
| 132 | D | K | D | K | D | N | V | A | P | R | T | K | V | S | P | Q | G | Hyp |
| 133 | D | K | D | K | D | P | V | A | P | P | T | N | V | G | S | N | G | Hyp |
| 134 | D | K | D | K | D | N | V | A | P | R | T | K | V | S | P | N | G | Hyp |
| 135 | D | K | D | K | D | N | V | A | P | R | S | N | V | G | P | Q | G | Hyp |
| 136 | D | K | D | K | D | N | V | A | P | P | T | N | V | G | S | N | G | Hyp |
| 137 | D | K | D | K | D | P | V | A | P | P | T | N | V | G | S | N | G | Hyp |
| 138 | D | K | D | K | D | N | V | A | P | R | S | N | V | G | P | Q | G | Y |
| 139 | D | K | D | K | D | N | V | A | P | R | S | K | V | G | P | N | G | Hyp |
| 140 | D | K | D | K | D | N | V | A | P | R | T | N | I | S | P | Q | G | Y |
| 141 | D | K | D | K | D | N | V | A | P | R | T | N | I | S | P | Q | G | Hyp |
| 142 | D | K | D | K | D | N | V | A | P | R | T | N | I | G | P | N | G | Y |
| 143 | D | K | D | K | D | N | V | A | P | R | T | K | V | G | P | Q | G | Hyp |
| 144 | D | K | D | K | D | P | V | A | P | P | T | N | V | G | S | N | G | Hyp |
| 145 | D | K | D | K | D | N | V | A | P | R | S | N | V | S | P | Q | G | Hyp |
| 146 | D | K | D | K | D | N | V | A | P | R | T | K | I | S | P | Q | G | Hyp |
| 147 | D | K | F | K | D | N | V | A | P | R | S | K | I | S | P | Q | T | Hyp |
| 148 | N | N | F | G | A | — | I | L | S | S | T | K | I | G | S | N | G | Y |
| 149 | N | N | F | G | A | — | I | L | P | S | T | K | V | G | P | N | G | Y |
| 150 | D | K | D | K | D | N | V | A | P | R | S | K | I | S | P | Q | T | Hyp |
| 151 | D | K | D | K | D | N | V | A | P | R | S | N | I | G | P | N | G | Hyp |
| 152 | D | K | D | K | D | N | V | A | P | R | S | K | V | G | P | Q | G | Hyp |
| 153 | D | K | D | K | D | N | V | A | P | P | T | N | V | G | S | N | G | Hyp |
| 154 | D | K | D | K | D | N | V | A | P | R | S | K | I | S | P | Q | G | Y |
| 155 | D | K | D | K | D | N | V | A | P | R | T | K | V | S | P | Q | G | Y |
| 156 | D | K | D | K | D | N | V | A | P | R | T | K | V | G | P | Q | G | Y |
| 157 | D | K | D | K | D | N | V | A | P | R | S | K | I | G | P | N | G | Hyp |
| 158 | D | K | D | K | D | N | V | A | P | R | S | N | I | S | P | Q | G | Hyp |
| 159 | D | K | D | K | D | N | V | A | P | P | T | N | V | G | S | N | G | Hyp |
| 160 | D | K | D | K | D | N | V | A | P | R | T | N | I | G | P | Q | G | Y |
| 161 | D | K | D | K | D | N | V | A | P | R | T | K | I | G | P | Q | G | Hyp |
| 162 | N | N | F | G | A | — | I | L | P | S | T | K | I | G | P | N | G | Y |
| 163 | D | K | D | K | D | N | V | A | P | R | T | N | V | S | P | N | G | Hyp |
| 164 | D | K | D | K | D | N | V | A | P | R | T | K | I | S | P | Q | G | Y |
| 165 | D | K | D | K | D | N | V | A | P | R | S | N | I | G | P | Q | G | Hyp |
| 166 | D | K | D | K | D | N | V | A | P | R | S | N | V | S | P | N | G | Hyp |
| 167 | D | K | D | K | D | N | V | A | P | R | S | K | V | S | P | Q | G | Hyp |
| 168 | D | K | D | K | D | N | V | A | P | R | S | K | I | S | P | Q | T | Hyp |
| 169 | D | K | D | K | D | N | V | A | P | R | S | K | I | S | P | Q | G | Hyp |
| 170 | D | K | D | K | D | N | V | A | P | R | T | N | I | S | P | N | G | Hyp |
| 171 | D | K | F | K | D | N | V | A | P | R | S | K | I | S | P | Q | T | Hyp |
| 172 | D | K | D | K | D | N | V | A | P | R | S | N | V | S | P | N | G | Y |
| 173 | D | K | F | K | D | N | V | A | P | R | S | K | I | S | P | Q | T | Hyp |
| 174 | D | K | D | K | D | N | V | A | P | R | S | K | V | S | P | Q | G | Y |
| 175 | D | K | D | K | D | N | V | A | P | R | S | K | V | S | P | N | G | Hyp |
| 176 | D | K | D | K | D | N | V | A | P | R | T | N | V | S | P | Q | G | Y |
| 177 | D | K | D | K | D | N | V | A | P | R | S | N | V | G | P | N | G | Y |
| 178 | D | K | D | K | D | N | V | A | P | R | T | N | V | S | P | Q | G | Hyp |
| 179 | D | K | D | K | D | N | V | A | P | R | S | K | I | G | P | Q | G | Hyp |
| 180 | N | N | F | G | A | — | I | L | P | S | T | K | I | G | S | N | T | Y |
| 181 | D | K | D | K | D | N | V | A | P | P | T | N | V | G | S | N | G | Hyp |
| 182 | D | K | D | K | D | N | V | A | P | R | S | N | I | S | P | Q | G | Y |
| 183 | D | K | D | K | D | N | V | A | P | R | S | N | V | S | P | Q | G | Y |
| 184 | D | K | F | K | D | N | V | A | P | R | S | K | I | S | P | Q | T | W |
| 185 | D | K | F | K | D | N | V | A | P | R | S | K | I | S | P | Q | W | Hyp |
| 186 | D | K | F | K | D | N | V | A | P | R | S | K | I | S | P | Q | Y | HYP |
| 187 | D | K | D | K | D | N | V | A | P | R | S | K | V | G | P | Q | G | Hyp |
| 188 | D | K | D | K | D | N | V | A | P | R | T | N | I | S | P | N | G | Y |
| 189 | D | K | D | K | D | N | V | A | P | R | T | K | I | S | P | Q | G | Hyp |
| 190 | D | K | D | K | D | N | V | A | P | R | T | K | I | S | P | N | G | Y |
| 191 | D | K | D | K | D | N | V | A | P | R | S | N | I | G | P | Q | G | Y |
| 192 | N | K | D | K | D | N | V | A | P | R | S | K | I | S | P | Q | T | Hyp |
| 193 | D | K | F | K | D | N | V | A | P | R | S | K | I | S | P | Q | T | Hyp |
| 194 | D | K | D | K | D | N | V | A | P | R | S | K | I | S | P | Q | T | Hyp |
| 195 | D | K | D | K | D | N | V | A | P | P | T | N | V | G | S | N | G | Hyp |
| 196 | D | K | F | K | D | N | V | A | P | R | S | K | I | S | P | Q | T | Hyp |
| 197 | D | K | D | K | D | N | V | A | P | R | T | K | I | S | P | N | G | Hyp |
| 198 | N | K | D | K | D | N | V | A | P | R | S | K | I | S | P | Q | T | Hyp |
| 199 | D | K | D | K | D | N | V | A | P | R | T | N | I | S | P | Q | G | Y |
| 200 | D | K | D | K | D | N | V | A | P | R | T | N | V | G | P | Q | G | Y |
| 201 | D | K | F | K | D | N | V | A | P | R | S | K | I | S | P | Q | H | Hyp |
| 202 | D | K | D | K | D | N | V | A | P | S | S | K | I | S | P | Q | T | Hyp |
| 203 | D | K | F | K | D | N | V | A | P | R | S | K | I | S | P | Q | P | Hyp |
| 204 | D | K | D | K | D | N | V | A | P | R | S | K | V | S | P | N | G | Hyp |
| 205 | D | K | D | K | D | N | V | A | P | R | S | K | I | S | P | Q | T | Hyp |
| 206 | D | K | F | K | D | N | V | A | P | R | S | K | I | S | P | Q | K | Hyp |
| 207 | N | N | F | G | A | — | I | L | P | S | T | K | I | G | S | N | G | Y |
| 208 | D | K | D | K | D | N | V | A | P | R | T | N | V | G | S | N | T | Y |
| 209 | D | K | D | K | D | N | V | A | P | R | T | K | V | S | P | Q | G | Y |
| 210 | D | K | D | K | D | N | V | A | P | P | T | N | V | G | S | N | G | Hyp |
| 211 | D | K | F | K | D | N | V | A | P | R | S | K | I | S | P | Q | F | Hyp |
| 212 | D | K | F | K | D | N | V | A | P | R | S | K | I | S | P | Q | T | Hyp |
| 213 | D | K | F | K | D | N | V | A | P | R | S | K | I | S | P | Q | T | Hyp |
| 214 | D | K | D | K | D | N | V | A | P | R | T | N | I | S | P | Q | G | Hyp |
| 215 | D | K | F | K | D | N | V | A | P | R | S | K | I | S | P | Q | T | M |
| 216 | D | K | D | K | D | N | V | A | P | R | T | K | V | S | P | Q | G | Hyp |
| 217 | D | K | D | K | D | N | V | A | P | R | S | K | I | S | P | Q | T | Hyp |
| 218 | D | K | F | K | D | N | V | A | P | R | S | K | I | S | P | Q | A | Hyp |
| 219 | N | K | D | K | D | N | V | A | P | R | S | K | I | S | P | Q | H | Hyp |
| 220 | N | K | D | K | D | N | V | A | P | R | S | K | I | S | P | Q | W | Hyp |
| 221 | D | K | D | K | D | N | V | A | P | P | T | N | V | G | S | N | G | Hyp |
| 222 | D | K | D | K | D | N | V | A | P | S | S | K | I | S | P | Q | T | Hyp |
| 223 | D | K | D | K | D | N | V | A | P | R | T | K | V | G | S | N | G | Y |
| 224 | D | K | D | K | D | N | V | A | P | R | S | N | V | S | P | Q | G | Hyp |
| 225 | D | K | D | K | D | N | V | A | P | P | T | N | V | G | S | N | G | Hyp |
| 226 | N | K | D | K | D | N | V | A | P | R | S | K | I | S | P | Q | T | W |
| 227 | N | K | D | K | D | N | V | A | P | R | S | K | I | S | P | Q | T | Hyp |
| 228 | D | K | D | K | D | N | V | A | P | R | S | K | I | S | P | Q | T | Hyp |
| 229 | D | K | D | K | D | N | V | A | P | R | S | N | I | G | P | Q | G | Hyp |
| 230 | D | K | F | K | D | N | V | A | P | R | S | K | I | S | P | Q | T | Y |
| 231 | D | K | D | K | D | N | V | A | P | P | T | N | V | G | S | N | G | Hyp |
| 232 | N | K | D | K | D | N | V | A | P | R | S | K | I | S | P | Q | Q | Hyp |
| 233 | D | K | D | K | D | N | V | A | P | P | T | N | V | G | S | N | G | Hyp |
| 234 | D | K | D | K | D | N | V | A | P | R | S | K | I | S | P | Q | G | Y |
| 235 | D | K | F | K | D | N | V | A | P | R | S | K | I | S | P | Q | T | P |
| 236 | D | K | F | K | D | N | V | A | P | R | S | K | I | S | P | Q | M | Hyp |
| 237 | N | K | D | K | D | N | V | A | P | R | S | K | I | S | P | Q | T | M |
| 238 | N | K | D | K | D | N | V | A | P | R | S | K | I | S | P | Q | F | Hyp |
| 239 | D | K | F | K | D | N | V | A | P | R | S | K | I | S | P | Q | T | F |
| 240 | D | K | D | K | D | N | V | A | P | S | S | K | I | S | P | Q | T | Hyp |
| 241 | D | K | F | K | D | N | V | A | P | R | S | K | I | S | P | Q | T | A |
| 242 | N | K | D | K | D | N | V | A | P | R | S | K | I | S | P | Q | T | R |
| 243 | D | K | D | K | D | N | V | A | P | P | T | N | V | G | S | N | G | Hyp |
| 244 | N | K | D | K | A | Q | V | S | P | P | T | E | V | G | P | N | S | Hyp |
| 245 | N | K | D | K | A | Q | V | S | P | P | T | E | V | G | P | N | S | Hyp |
| 246 | D | K | D | K | D | N | V | A | P | P | T | N | V | G | S | N | G | Hyp |
| 247 | D | K | D | K | A | N | V | S | P | P | T | E | V | G | P | N | S | Hyp |
| 248 | D | K | D | K | D | N | V | A | P | R | T | K | V | G | S | N | G | Hyp |
| 249 | D | K | D | K | D | N | V | A | P | P | T | N | V | G | S | N | G | Hyp |
| 250 | D | K | D | A | D | N | V | A | P | P | T | N | V | G | S | N | G | Hyp |
| 251 | D | K | D | K | D | N | A | A | P | P | T | N | V | G | S | N | G | Hyp |
| 252 | D | K | D | K | D | N | V | A | P | A | T | N | V | G | S | N | G | Hyp |
| 253 | T | K | D | K | D | N | V | A | P | P | T | N | V | G | S | N | G | Hyp |
| 254 | D | K | D | K | D | N | V | P | P | T | — | N | V | G | S | N | G | Hyp |
| 255 | D | K | D | K | D | N | V | A | P | T | — | N | V | G | S | N | G | Hyp |
| 256 | D | K | D | K | D | Q | V | A | P | P | T | N | V | G | S | N | S | Hyp |
| 257 | D | K | D | K | D | N | V | A | P | R | T | K | V | G | S | N | G | Y |
| 258 | D | K | A | K | D | N | V | A | P | P | T | N | V | G | S | N | G | Hyp |
| 259 | D | K | D | K | D | N | V | A | P | R | T | K | V | G | P | Q | G | Hyp |
| 260 | D | K | D | K | D | N | V | A | P | R | T | N | I | G | P | Q | G | Hyp |
| 261 | D | K | D | K | D | N | V | A | P | R | T | N | V | G | P | Q | G | Hyp |
| 262 | D | K | D | K | D | N | V | A | A | P | T | N | V | G | S | N | G | Hyp |
| 263 | D | K | D | K | D | N | V | A | P | R | T | N | V | G | P | Q | G | Y |
| 264 | D | K | D | K | D | N | V | A | P | R | T | N | V | G | S | N | T | Y |
| 265 | D | K | D | K | D | N | V | A | P | P | T | N | V | G | S | N | G | Hyp |
| 266 | D | K | D | K | D | N | V | S | P | P | T | N | V | G | S | N | G | Hyp |
| 267 | D | K | D | K | D | N | V | A | P | R | T | N | V | G | P | Q | G | Hyp |
| 268 | D | K | D | K | D | N | V | A | P | P | T | N | V | G | S | N | G | Hyp |
| 269 | D | K | D | K | D | N | V | A | P | R | T | N | V | G | P | N | G | Y |
| 270 | D | K | D | K | D | N | V | A | P | R | T | N | V | G | P | N | G | Hyp |
| 271 | D | K | D | K | D | N | V | A | P | R | T | N | V | S | P | N | G | Y |
| 272 | D | K | D | K | D | N | V | A | P | R | T | E | V | G | S | N | G | Y |
| 273 | D | K | D | K | D | N | V | A | P | R | T | N | V | G | P | Q | G | Y |
| 274 | D | K | D | K | D | N | V | A | P | R | T | K | I | G | P | Q | G | Hyp |
| 275 | D | K | D | K | D | N | V | A | P | R | T | K | I | S | P | N | G | Hyp |
| 276 | D | K | D | K | D | N | V | A | P | P | T | N | V | G | S | N | G | Hyp |
| 277 | D | K | D | K | D | N | V | A | P | R | T | K | I | G | P | N | G | Hyp |
| 278 | D | K | D | K | D | N | V | A | P | R | T | N | V | S | P | Q | G | Y |
| 279 | D | K | D | K | D | N | V | A | P | R | T | K | V | G | S | Z | G | Y |
| 280 | D | K | D | K | D | N | V | A | P | R | T | N | I | G | P | Q | G | Hyp |
| 281 | D | K | D | K | D | N | V | A | P | R | S | N | V | G | P | Q | G | Hyp |
| 282 | D | K | D | G | — | A | I | A | P | R | T | K | I | G | S | O | G | Y |
| 283 | D | K | D | K | D | N | V | A | P | R | T | N | V | S | P | Q | G | Hyp |
| 284 | D | K | D | K | D | N | V | A | P | R | S | K | I | G | P | Q | G | Hyp |
| 285 | D | K | D | K | D | N | V | A | P | R | S | K | V | G | P | Q | G | Y |
| 286 | D | K | D | K | D | N | V | A | P | R | S | N | V | S | P | Q | G | Y |
| 287 | D | K | D | K | D | N | V | A | P | R | T | K | V | S | P | Q | G | Hyp |
| 288 | D | K | D | K | D | N | V | A | P | R | T | K | V | S | P | N | G | Hyp |
| 289 | D | K | D | K | D | N | V | A | P | R | S | N | V | G | P | Q | G | Hyp |
| 290 | D | K | D | K | D | N | V | A | P | R | S | K | V | G | P | N | G | Hyp |
| 291 | D | K | D | K | D | N | V | A | P | R | T | N | I | S | P | Q | G | Hyp |
| 292 | D | K | D | K | D | N | V | A | P | R | T | K | V | G | P | Q | G | Hyp |
| 293 | D | K | D | K | D | N | V | A | P | R | S | N | V | S | P | Q | G | Hyp |
| 294 | D | K | D | K | D | N | V | A | P | R | T | K | I | S | P | Q | G | Hyp |
| 295 | D | K | F | K | D | N | V | A | P | R | S | K | I | S | P | Q | T | Hyp |
| 296 | D | K | D | K | D | N | V | A | P | R | S | K | I | S | P | Q | T | Hyp |
| 297 | D | K | D | K | D | N | V | A | P | R | S | N | I | G | P | N | G | Hyp |
| 298 | D | K | D | K | D | N | V | A | P | R | T | K | V | G | P | Q | G | Y |
| 299 | D | K | D | K | D | N | V | A | P | R | S | K | I | G | P | N | G | Hyp |
| 300 | D | K | D | K | D | N | V | A | P | R | S | N | I | S | P | Q | G | Hyp |
| 301 | D | K | D | K | D | N | V | A | P | R | T | K | I | G | P | Q | G | Hyp |
| 302 | D | K | D | K | D | N | V | A | P | R | T | N | V | S | P | N | G | Hyp |
| 303 | D | K | D | K | D | N | V | A | P | R | S | N | I | G | P | Q | G | Hyp |
| 304 | D | K | D | K | D | N | V | A | P | R | S | N | V | S | P | N | G | Hyp |
| 305 | D | K | D | K | D | N | V | A | P | R | S | K | V | S | P | Q | G | Hyp |
| 306 | N | K | D | K | A | Q | V | S | P | P | T | E | V | G | P | N | S | Hyp |
| 307 | N | K | D | K | A | 0 | V | S | P | P | T | E | V | G | P | N | S | Hyp |
| 308 | D | K | D | K | D | N | V | A | P | P | T | N | V | G | S | N | G | Hyp |
| 309 | D | K | D | K | D | N | V | A | P | P | T | N | V | G | S | N | G | Hyp |
| 310 | D | K | D | K | A | N | V | S | P | P | T | E | V | G | P | N | S | Hyp |
| 311 | D | K | D | K | D | N | V | A | P | R | T | N | V | G | P | Q | G | Y |
| 312 | D | K | D | K | D | N | V | A | P | R | T | K | V | G | S | N | G | Hyp |
| 313 | D | K | D | K | D | N | V | A | P | P | T | N | V | G | S | N | G | Hyp |
| 314 | D | K | D | K | A | N | V | A | P | P | T | N | V | G | S | N | G | Hyp |
| 315 | D | K | D | K | D | N | A | A | P | P | T | N | V | G | S | N | G | Hyp |
| 316 | D | K | D | K | D | N | V | A | P | A | T | N | V | G | S | N | G | Hyp |
| 317 | T | K | D | K | D | N | V | A | P | P | T | N | V | G | S | N | G | Hyp |
| 318 | D | K | D | K | D | N | V | P | P | T | — | N | V | G | S | N | G | Hyp |
| 319 | D | K | D | K | D | N | V | A | P | T | — | N | V | G | S | N | G | Hyp |
| 320 | D | K | A | K | D | N | V | A | P | P | T | N | V | G | S | N | G | Hyp |
| 321 | D | K | D | K | D | N | V | A | P | P | Y | N | E | G | S | N | G | Hyp |
| 322 | D | K | D | K | D | N | V | A | P | R | T | K | V | G | P | Q | G | Hyp |
| 323 | D | K | D | K | D | N | V | A | P | R | T | N | V | G | P | Q | G | Hyp |
| 324 | D | K | D | K | D | A | V | A | P | P | T | N | V | G | S | N | G | Hyp |
| 325 | D | K | D | K | D | N | V | A | A | P | T | N | V | G | S | N | G | Hyp |
| 326 | D | K | D | K | D | N | V | A | P | R | T | N | V | G | P | Q | G | Y |
| 327 | D | K | D | K | D | N | V | A | P | R | T | N | V | G | S | N | T | Y |
| 328 | D | A | D | K | D | N | V | A | P | P | T | N | V | G | S | N | G | Hyp |
| 329 | D | K | D | K | D | N | V | A | P | P | T | N | V | G | S | N | G | Hyp |
| 330 | D | K | D | K | D | N | V | A | P | P | T | N | V | G | S | N | G | Hyp |
| 331 | D | K | D | K | D | N | V | A | P | R | T | N | I | G | P | N | G | Hyp |
| 332 | D | K | D | K | D | N | V | A | P | R | T | N | V | G | P | N | G | Y |
| 333 | D | K | D | K | D | N | V | A | P | P | T | N | V | G | S | N | G | Hyp |
| 334 | D | K | D | K | D | N | V | A | P | R | T | E | V | G | S | N | G | Y |
| 335 | D | K | D | K | D | N | V | A | P | R | T | N | V | G | P | Q | G | Y |
| 336 | D | K | D | K | D | N | V | A | P | R | T | K | I | G | P | Q | G | Hyp |
| 337 | D | K | D | K | D | N | V | A | P | R | T | K | I | S | P | N | G | Hyp |
| 338 | D | K | D | K | D | N | V | A | P | P | T | N | V | G | S | N | G | Hyp |
| 339 | D | K | D | K | D | N | V | A | P | R | T | K | I | G | P | N | G | Hyp |
| 340 | D | K | D | K | D | N | V | A | P | R | T | N | V | S | P | Q | G | Y |
| 341 | D | K | D | K | D | N | V | A | P | R | T | N | I | G | P | Q | G | Hyp |
| 342 | D | K | D | K | D | N | V | A | P | R | S | N | V | G | P | Q | G | Hyp |
| 343 | D | K | D | K | D | N | V | A | P | R | T | K | I | G | P | N | G | Hyp |
| 344 | D | K | D | K | D | N | V | A | P | R | S | K | V | G | P | Q | G | Y |
| 345 | D | K | D | K | D | N | V | A | P | R | S | N | V | S | P | Q | G | Y |
| 346 | D | K | D | K | D | N | V | A | P | R | S | N | V | G | P | Q | G | Y |
| 347 | D | K | D | K | D | N | V | A | P | R | S | K | V | G | P | N | G | Hyp |
| 348 | D | K | D | K | D | N | V | A | P | R | T | N | I | G | P | N | G | Y |
| 349 | D | K | D | K | D | N | V | A | P | R | T | K | V | G | P | Q | G | Hyp |
| 350 | D | K | D | K | D | N | V | A | P | R | T | K | I | S | P | Q | G | Hyp |
| 351 | D | K | F | K | D | N | V | A | P | R | S | K | I | S | P | Q | T | Hyp |
| 352 | D | K | D | K | D | N | V | A | P | R | S | N | I | G | P | N | G | Hyp |
| 353 | D | K | D | K | D | N | V | A | P | R | S | K | V | G | P | Q | G | Hyp |
| 354 | D | K | D | K | D | N | V | A | P | R | T | K | V | G | P | Q | G | Y |
| 355 | D | K | D | K | D | N | V | A | P | R | S | K | I | G | P | N | G | Hyp |
| 356 | D | K | D | K | D | N | V | A | P | R | T | N | I | G | P | Q | G | Y |
| 357 | D | K | D | K | D | N | V | A | P | R | T | K | I | G | P | Q | G | Hyp |
| 358 | D | K | D | K | D | N | V | A | P | R | T | N | V | S | P | N | G | Hyp |
| 359 | D | K | D | K | D | N | V | A | P | R | S | N | I | G | P | Q | G | Hyp |
| 360 | D | K | D | K | D | N | V | A | P | R | S | N | V | S | P | N | G | Hyp |
| 361 | D | K | D | K | D | N | V | A | P | R | T | K | V | G | S | N | G | Y |
| 362 | D | K | D | K | D | N | V | A | P | R | T | N | V | G | P | Q | G | Y |
| 363 | D | K | D | K | D | N | V | A | P | R | S | K | I | G | P | Q | G | Y |
| 364 | D | K | D | K | D | N | V | A | P | R | T | N | I | G | P | Q | G | Y |
| 365 | D | K | D | K | D | N | V | A | P | R | T | K | I | G | P | Q | G | Y |
| 366 | D | K | D | K | D | N | V | A | P | R | T | N | I | S | P | N | G | Y |
| 367 | D | K | D | K | D | N | V | A | P | R | T | K | I | G | P | N | G | Y |
| 368 | D | K | D | K | D | N | V | A | P | R | T | K | V | G | P | Q | G | Y |
| 369 | D | K | D | K | D | N | V | A | P | R | T | K | V | G | P | Q | G | Y |
| 370 | D | K | D | K | D | N | V | A | P | R | T | E | V | G | S | N | G | Y |
| 371 | D | K | D | K | D | N | V | A | P | R | T | K | I | S | P | N | G | Y |
| 372 | D | K | D | K | D | N | V | A | P | R | T | N | V | G | P | Q | G | Y |
| 373 | D | K | D | K | D | N | V | A | P | R | S | N | V | G | P | N | G | Y |
| 374 | D | K | D | K | D | N | V | A | P | R | S | N | V | G | P | Q | G | Y |
| 375 | D | K | D | K | D | N | V | A | P | R | S | K | V | G | P | Q | G | Y |
| 376 | D | K | D | K | D | N | V | A | P | R | T | K | I | G | S | N | G | Y |
| 377 | D | K | D | K | D | N | V | A | P | R | T | K | V | G | P | Q | G | Y |
| 278 | D | K | D | K | D | N | V | A | P | R | T | K | V | G | S | N | G | Y |
| 379 | D | K | D | K | D | N | V | A | P | R | T | N | I | G | P | Q | G | Y |
| 380 | D | K | D | K | D | N | V | A | P | R | T | K | V | G | P | Q | G | Y |
| 381 | D | K | D | K | D | N | V | A | P | R | T | N | I | G | P | N | G | Y |
| 382 | D | K | D | K | D | N | V | A | P | R | S | N | I | G | P | Q | G | Y |
| 383 | D | K | D | K | D | N | V | A | P | R | S | N | I | S | P | N | G | Y |
| 384 | D | K | D | K | D | N | V | A | P | R | S | K | V | S | P | N | G | Y |
| 385 | D | K | D | K | D | N | V | A | P | R | T | K | V | S | P | N | G | Y |
| 386 | D | K | D | K | D | N | V | A | P | R | T | N | V | G | P | Q | G | Y |
| 387 | D | K | D | K | D | N | V | A | P | R | S | K | I | G | P | Q | G | Y |
| 388 | D | K | D | K | D | N | V | A | P | R | T | N | I | G | P | Q | G | Y |
| 389 | D | K | D | K | D | N | V | A | P | R | S | K | I | G | P | Q | G | Y |
| 390 | D | K | D | K | D | N | V | A | P | R | T | K | I | G | P | Q | G | Y |
| 391 | D | K | D | K | D | N | V | A | P | R | T | K | I | G | P | Q | G | Y |
| 392 | D | K | D | K | D | N | V | A | P | R | T | K | V | G | P | Q | G | Y |

1-14. (canceled)

15. An hAM₁₅₋₅₂ analogue or a pharmaceutically acceptable salt thereof comprising 38 amino acids (X₁-X₃₈) with an hAMY3R-EC₅₀≤250 pM and an hAM1R-EC₅₀≥25 nM, wherein the amino acid in position X₄ is selected as F, Y, W, T, M, I, A, or C; X₃₇ is selected as G, Y, S, W, T, Q, P, M, I, H, F, E, A, R, C, or K; X₃₈ is selected as Hyp, Y, W, T, Q, P, M, I, H, F, E, A, R, or K; X₁₁ is R, W, or Cit and wherein at least one of the positions X₄, X₃₇ or X₃₈ is not the amino acid present in hAM₁₅₋₅₂ (SEQ ID NO: 1) in said position and further wherein the hAM₁₅₋₅₂ analogue has at least 50% identity to hAM₁₅₋₅₂ (SEQ ID NO: 1).

16. An hAM₁₅₋₅₂ analogue or a pharmaceutically acceptable salt thereof according to claim 15, wherein the hAM₁₅₋₅₂ analogue has at least 60% identity to hAM₁₅₋₅₂, such as at least 70% identity to hAM₁₅₋₅₂, preferably at least 80% identity to hAM₁₅₋₅₂ (SEQ ID NO: 1).

17. An hAM₁₅₋₅₂ analogue or a pharmaceutically acceptable salt thereof according to claim 15, comprising the amino acid sequence of formula (I): 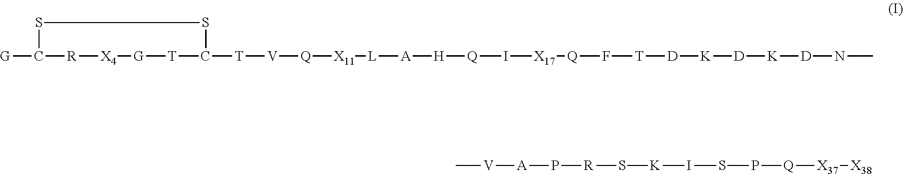
wherein
X₁₁ is selected from R, W, or Cit;
X₁₇ is selected as Y, W, T, Q, M, I, H, F, A, R, or K;
X₄ is selected from F, Y, W, T, M, I, A, or C;
X₃₇ is selected from G, Y, S, W, T, Q, P, M, I, H, F, E, A, R, C, or K;
X₃₈ is selected from Hyp, Y, W, T, Q, P, M, I, H, F, E, A, R, or K;
and further wherein at least one of the positions X₄, X₃₇ or X₃₈ is not the amino acid present in hAM₁₅₋₅₂ (SEQ ID NO: 1) in said position;
or a derivative thereof with at least 50% identity to hAM₁₅₋₅₂, such as at least 55% identity to hAM₁₅₋₅₂, such as at least 60% identity to hAM₁₅₋₅₂, such as at least 65% identity to hAM₁₅₋₅₂, preferably at least 70% identity to hAM₁₅₋₅₂, such as at least 75% identity to hAM₁₅₋₅₂, more preferably at least 80% identity to hAM₁₅₋₅₂, such as at least 90% identity to hAM₁₅₋₅₂, preferably at least 95% identity to hAM₁₅₋₅₂, wherein, in the derivative, X₁₁ is selected from R, W, or Cit; X₁₇ is selected as Y, W, T, Q, M, I, H, F, A, R, or K; X₄ is selected from F, Y, W, T, M, I, A or C; X₃₇ is selected from G, Y, S, W, T, Q, P, M, I, H, F, E, A, R, C, or K; X₃₈ is selected from Hyp, Y, W, T, Q, P, M, I, H, F, E, A, R, or K; and further wherein at least one of the positions X₄, X₃₇ or X₃₈ is not the amino acid present in hAM₁₅₋₅₂ (SEQ ID NO: 1) in said position.

18. (canceled)

19. An hAM₁₅₋₅₂ analogue or a pharmaceutically acceptable salt thereof according to claim 15 with the sequence SEQ ID NOs: 3-57, 60-75, 77-79, 81-92, 94-127, 129-147, 150-161, 163-179, 181-206, 208-243, 246-305, or 308-360 or a derivative thereof with at least 90% identity to any one of SEQ ID NO: 3-57, 60-75, 77-79, 81-92, 94-127, 129-147, 150-161, 163-179, 181-206, 208-243, 246-305, or 308-360, preferably at least 95% identity to any one of SEQ ID NO: 3-57, 60-75, 77-79, 81-92, 94-127, 129-147, 150-161, 163-179, 181-206, 208-243, 246-305, or 308-360, most preferably at least 97% identity to anyone of SEQ ID NOs: 3-57, 60-75, 77-79, 81-92, 94-127, 129-147, 150-161, 163-179, 181-206, 208-243, 246-305, or 308-360, with the proviso that in the derivative X₁₁ is selected from R, W, or Cit; X₁₇ is selected as Y, W, T, Q, M, I, H, F, A, R or K; X₄ is selected from F, Y, W, T, M, I, A, or C; X₃₇ is selected from G, Y, S, W, T, Q, P, M, I, H, F, E, A, R, C, or K; X₃₈ is selected from Hyp, Y, W, T, Q, P, M, I, H, F, E, A, R, or K; and further wherein at least one of the positions X₄, X₃₇ or X₃₈ is not the amino acid present in hAM₁₅₋₅₂ (SEQ ID NO: 1) in said position.

20. An hAM₁₅₋₅₂ analogue or a derivative thereof according to claim 15, wherein at least two of the positions X₄, X₃₇, and X₃₈ is not the amino acid present in hAM₁₅₋₅₂ (SEQ ID NO: 1) in said position.

21. An hAM₁₅₋₅₂ analogue or a derivative thereof according to claim 15, wherein all of the positions X₄, X₃₇ and X₃₈ are selected as follows:
X₄ is selected from Y, W, T, M, I, A, or C;
X₃₇ is selected from Y, S, W, T, Q, P, M, I, H, F, E, A, R, C, or K;
X₃₈ is selected from Hyp, W, T, Q, P, M, I, H, F, E, A, R, or K;

22. An hAM₁₅₋₅₂ analogue or a derivative thereof according to claim 15, wherein X₄ is selected as F, W, M, I, or C.

23. An hAM₁₅₋₅₂ analogue or a derivative thereof according to claim 15, wherein X₃₇ is selected as G, Y, W, P, H, or F.

24. An hAM₁₅₋₅₂ analogue or a derivative thereof according to claim 15, wherein X₃₈ is selected as Hyp, Y, W, M, or F.

25. An hAM₁₅₋₅₂ analogue or a derivative thereof according to claim 15, wherein X₁₇ is selected as W, or H.

26. An hAM₁₅₋₅₂ analogue or a derivative thereof according to claim 15, wherein X₁₁ is R.

27. An hAM₁₅₋₅₂ analogue or a derivative thereof according to claim 15 for use as a medicament.

28. A pharmaceutical composition comprising an hAM₁₅₋₅₂ analogue or a derivative thereof according to claim 15, a pharmaceutically acceptable carrier and optionally one or more excipient(s).


SEQUENCE LISTING (392 sequences)

SEQ ID NO: 1 - protein, 38 aa, Artificial Sequence
  Designed polypeptide
GCRFGTCTVQKLAHQIYQFTDKDKDNVAPRSKISPQGY

SEQ ID NO: 2 - protein, 37 aa, Artificial Sequence [sequence omitted]

SEQ ID NO: 3 - protein, 38 aa, Artificial Sequence
  Designed polypeptide
  misc_feature (38)..(38): X = Hyp (L-hydroxyproline)
KCNFATCTVQRLAHQIYQFTDKDKDNVAPRTKVGSNGX

SEQ ID NO: 4 - protein, 38 aa, Artificial Sequence
  Designed polypeptide
  misc_feature (38)..(38): X = Hyp (L-hydroxyproline)
KCNTATCTVDRLAHQIYQFTDKDKDNVAPPTNVGSNGX

SEQ ID NO: 5 - protein, 38 aa, Artificial Sequence
  Designed polypeptide
  misc_feature (38)..(38): X = Hyp (L-hydroxyproline)
KCNTATCTVQRLADQIYQFTDKDKDNVAPPTNVGSNGX

SEQ ID NO: 6 - protein, 38 aa, Artificial Sequence
  Designed polypeptide
  misc_feature (38)..(38): X = Hyp (L-hydroxyproline)
KCNTATCTVQRLAEQIYQFTDKDKDNVAPPTNVGSKGX

SEQ ID NO: 7 - protein, 38 aa, Artificial Sequence
  Designed polypeptide
  misc_feature (38)..(38): X = Hyp (L-hydroxyproline)
KCNTATCTVQRLAAQIYQFTDKDKDNVAPPTNVGSNGX

SEQ ID NO: 8 - protein, 38 aa, Artificial Sequence
  Designed polypeptide
  misc_feature (38)..(38): X = Hyp (L-hydroxyproline)
KCNTSTCTVARLADQITQFSNKDKAQVSPPTEVGPNSX

SEQ ID NO: 9 - protein, 38 aa, Artificial Sequence
  Designed polypeptide
  misc_feature (38)..(38): X = Hyp (L-hydroxyproline)
RCNASTCTVNRLADQITQFSNKDKAQVSPPTEVGPNSX

SEQ ID NO: 10 - protein, 38 aa, Artificial Sequence
  Designed polypeptide
  misc_feature (38)..(38): X = Hyp (L-hydroxyproline)
KCNFATCTVQRLAHQIYQFTDKDKDNVAPPTEVGSNGX

SEQ ID NO: 11 - protein, 38 aa, Artificial Sequence
  Designed polypeptide
  misc_feature (38)..(38): X = Hyp (L-hydroxyproline)
KCNTATCTVQRLAHQINQFTDKDKDNVAPPTNVGSNGX

SEQ ID NO: 12 - protein, 38 aa, Artificial Sequence
  Designed polypeptide
  misc_feature (38)..(38): X = Hyp (L-hydroxyproline)
KCNTATCTVQRLAEQIYQFTDKDKDNKAPPTNVGSNGX

SEQ ID NO: 13 - protein, 38 aa, Artificial Sequence
  Designed polypeptide
  misc_feature (38)..(38): X = Hyp (L-hydroxyproline)
KCNTATCTVQRLAHAIYQFTDKDKDNVAPPTNVGSNGX

SEQ ID NO: 14 - protein, 38 aa, Artificial Sequence
  Designed polypeptide
  misc_feature (38)..(38): X = Hyp (L-hydroxyproline)
KCNTATCTVQRLAHQIAQFTDKDKDNVAPPTNVGSNGX

SEQ ID NO: 15 - protein, 38 aa, Artificial Sequence
  Designed polypeptide
  misc_feature (38)..(38): X = Hyp (L-hydroxyproline)
KCNTSTCTVARLADQITQFSDKDKANVSPPTEVGPNSX

SEQ ID NO: 16 - protein, 38 aa, Artificial Sequence
  Designed polypeptide
GCRFGTCTVQRLANFLYQFTDKDKDNVAPRTNVGPQGY

SEQ ID NO: 17 - protein, 38 aa, Artificial Sequence
  Designed polypeptide
  misc_feature (38)..(38): X = Hyp (L-hydroxyproline)
KCNFATCTVQRLAHQIYQFTDKDKDNVAPRTEVGSNGX

SEQ ID NO: 18 - protein, 38 aa, Artificial Sequence
  Designed polypeptide
KCNFATCTVQRLAHQIYQFTDKDKDNVAPPTEVGSNGP

SEQ ID NO: 19 - protein, 37 aa, Artificial Sequence
  Designed polypeptide
KCNTATCATQRLANFLVHSSNNFGAILPRTKVGSNGY

SEQ ID NO: 20 - protein, 38 aa, Artificial Sequence
  Designed polypeptide
  misc_feature (38)..(38): X = Hyp (L-hydroxyproline)
KCNTATCTVQRLAHQIYQFTDKDKDNVAPRTKVGSNGX

SEQ ID NO: 21 - protein, 38 aa, Artificial Sequence
  Designed polypeptide
  misc_feature (38)..(38): X = Hyp (L-hydroxyproline)
KCNTATCTVQRLANQIYQFTDKDKDNVAPPTNVGSNGX

SEQ ID NO: 22 - protein, 38 aa, Artificial Sequence
  Designed polypeptide
  misc_feature (38)..(38): X = Hyp (L-hydroxyproline)
KCNTATCTVQRLAHQITQFTDKDKDNVAPPTNVGSNGX

SEQ ID NO: 23 - protein, 38 aa, Artificial Sequence
  Designed polypeptide
  misc_feature (38)..(38): X = Hyp (L-hydroxyproline)
KCNTATCTVQRLAHQIYQFADKDKDNVAPPTNVGSNGX

SEQ ID NO: 24 - protein, 38 aa, Artificial Sequence
  Designed polypeptide
  misc_feature (38)..(38): X = Hyp (L-hydroxyproline)
KCNTATCTVQRLAHQIYQFTAKDKDNVAPPTNVGSNGX

SEQ ID NO: 25 - protein, 38 aa, Artificial Sequence
  Designed polypeptide
  misc_feature (38)..(38): X = Hyp (L-hydroxyproline)
KCNTATCTVQRLAHQIYQFTDKDADNVAPPTNVGSNGX

SEQ ID NO: 26 - protein, 38 aa, Artificial Sequence
  Designed polypeptide
  misc_feature (38)..(38): X = Hyp (L-hydroxyproline)
KCNTATCTVQRLAHQIYQFTDKDKANVAPPTNVGSNGX

SEQ ID NO: 27 - protein, 38 aa, Artificial Sequence
  Designed polypeptide
  misc_feature (38)..(38): X = Hyp (L-hydroxyproline)
KCNTATCTVQRLAHQIYQFTDKDKDNAAPPTNVGSNGX

SEQ ID NO: 28 - protein, 38 aa, Artificial Sequence
  Designed polypeptide
  misc_feature (38)..(38): X = Hyp (L-hydroxyproline)
KCNTATCTVQRLAHQIYQFTDKDKDNVAPATNVGSNGX

SEQ ID NO: 29 - protein, 37 aa, Artificial Sequence
  Designed polypeptide
  misc_feature (37)..(37): X = Hyp (L-hydroxyproline)
KCNTATCTVQRLAHQIYQFTKDKDNVAPPTNVGSNGX

SEQ ID NO: 30 - protein, 37 aa, Artificial Sequence
  Designed polypeptide
  misc_feature (37)..(37): X = Hyp (L-hydroxyproline)
KCNTATCTVQRLAHQIYQFTDKDKDNVPPTNVGSNGX

SEQ ID NO: 31 - protein, 37 aa, Artificial Sequence
  Designed polypeptide
  misc_feature (37)..(37): X = Hyp (L-hydroxyproline)
KCNTATCTVQRLAHQIYQFTDKDKDNVAPTNVGSNGX

SEQ ID NO: 32 - protein, 38 aa, Artificial Sequence
  Designed polypeptide
  misc_feature (38)..(38): X = Hyp (L-hydroxyproline)
RCQTSTCTVARLAEQIAQYTDKDKDQVAPPTNVGSNSX

SEQ ID NO: 33 - protein, 38 aa, Artificial Sequence
  Designed polypeptide
  misc_feature (11)..(11): X = Cit (L-citrulline)
  misc_feature (38)..(38): X = Hyp (L-hydroxyproline)
RCQASTCTVAXLAEQIAQYTDKDKDQVAPPTEVGPNSX

SEQ ID NO: 34 - protein, 38 aa, Artificial Sequence
  Designed polypeptide
KCNTATCTVQRLAHQIYQFTDKDKDNVAPRTKVGSNGY

SEQ ID NO: 35 - protein, 38 aa, Artificial Sequence
  Designed polypeptide
  misc_feature (38)..(38): X = Hyp (L-hydroxyproline)
KCNTATCTVQRLAHQIQQFTDKDKDNVAPPTNVGSNGX

SEQ ID NO: 36 - protein, 38 aa, Artificial Sequence
  Designed polypeptide
  misc_feature (38)..(38): X = Hyp (L-hydroxyproline)
KCKTATCTVQRLAEQIYQFTDKDKDNVAPPTNVGSNGX

SEQ ID NO: 37 - protein, 38 aa, Artificial Sequence
  Designed polypeptide
  misc_feature (38)..(38): X = Hyp (L-hydroxyproline)
KCNTATCTVARLAHQIYQFTDKDKDNVAPPTNVGSNGX

SEQ ID NO: 38 - protein, 38 aa, Artificial Sequence
  Designed polypeptide
  misc_feature (38)..(38): X = Hyp (L-hydroxyproline)
KCNTATCTVNRLAHQIYQFTDKDKDNVAPPTNVGSNGX

SEQ ID NO: 39 - protein, 38 aa, Artificial Sequence
  Designed polypeptide
  misc_feature (38)..(38): X = Hyp (L-hydroxyproline)
RCNTATCTVQRLAHQIYQFTDKDKDNVAPPTNVGSNGX

SEQ ID NO: 40 - protein, 38 aa, Artificial Sequence
  Designed polypeptide
  misc_feature (38)..(38): X = Hyp (L-hydroxyproline)
KCNTATCTVQRLAEQIYQFTDKDKKNVAPPTNVGSNGX

SEQ ID NO: 41 - protein, 38 aa, Artificial Sequence
  Designed polypeptide
  misc_feature (38)..(38): X = Hyp (L-hydroxyproline)
KCNTATCTVQRLAEQIYQFTDKDKDKVAPPTNVGSNGX

SEQ ID NO: 42 - protein, 38 aa, Artificial Sequence
  Designed polypeptide
  misc_feature (38)..(38): X = Hyp (L-hydroxyproline)
KCNTATCTVQRLAHQIYAFTDKDKDNVAPPTNVGSNGX

SEQ ID NO: 43 - protein, 38 aa, Artificial Sequence
  Designed polypeptide
  misc_feature (38)..(38): X = Hyp (L-hydroxyproline)
KCNTATCTVQRLAHQIYQFTDKAKDNVAPPTNVGSNGX

SEQ ID NO: 44 - protein, 38 aa, Artificial Sequence
  Designed polypeptide
  misc_feature (38)..(38): X = Hyp (L-hydroxyproline)
KCNTATCTVQRLAEQIYQFTDKDKDNVAPPYNEGSNGX

SEQ ID NO: 45 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRTKVGPQGX

SEQ ID NO: 46 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRTNIGPQGX

SEQ ID NO: 47 - protein, 38 aa, Artificial Sequence
  Designed polypeptide
  misc_feature (38)..(38): X = Hyp (L-hydroxyproline)
KCNTATCTVQRLAYQIYQFTDKDKDNVAPPTNVGSNGX

SEQ ID NO: 48 - protein, 38 aa, Artificial Sequence
  Designed polypeptide
  misc_feature (38)..(38): X = Hyp (L-hydroxyproline)
KCNTATCTVQRLAHQIYQYTDKDKDNVAPPTNVGSNGX

SEQ ID NO: 49 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRTNVGPQGX

SEQ ID NO: 50 - protein, 38 aa, Artificial Sequence
  Designed polypeptide
  misc_feature (38)..(38): X = Hyp (L-hydroxyproline)
KCNTSTCTVQRLAHQIYQFTDKDKDNVAPPTNVGSNGX

SEQ ID NO: 51 - protein, 38 aa, Artificial Sequence
  Designed polypeptide
  misc_feature (38)..(38): X = Hyp (L-hydroxyproline)
KCNTATCTVQRLAEQIYQFTDKDKDNVAPPTNVGSNGX

SEQ ID NO: 52 - protein, 38 aa, Artificial Sequence
  Designed polypeptide
  misc_feature (38)..(38): X = Hyp (L-hydroxyproline)
KCNTATCTVQRLAHQIYQFTDKDKDAVAPPTNVGSNGX

SEQ ID NO: 53 - protein, 38 aa, Artificial Sequence
  Designed polypeptide
  misc_feature (38)..(38): X = Hyp (L-hydroxyproline)
KCNTATCTVQRLAHQIYQFTDKDKDNVAAPTNVGSNGX

SEQ ID NO: 54 - protein, 38 aa, Artificial Sequence
  Designed polypeptide
GCRFGTCTVQRLAHFLYQFTDKDKDNVAPRTNVGPQGY

SEQ ID NO: 55 - protein, 38 aa, Artificial Sequence
  Designed polypeptide
  misc_feature (38)..(38): X = Hyp (L-hydroxyproline)
KCNTATCTVERLAHQIYQFTDKDKDNVAPPTNVGSNGX

SEQ ID NO: 56 - protein, 38 aa, Artificial Sequence
  Designed polypeptide
GCRFGTCTVQRLAHFLYQFTDKDKDNVAPRTNVGSNTY

SEQ ID NO: 57 - protein, 38 aa, Artificial Sequence
  Designed polypeptide
  misc_feature (38)..(38): X = Hyp (L-hydroxyproline)
KCNTATCTVQRLAHQIYQFSDKDKDNVAPPTNVGSNGX

SEQ ID NO: 58 - protein, 37 aa, Artificial Sequence
  Designed polypeptide
KCNTATCATQRLANFLVHSSNNFGAILSSTKVGSNTY

SEQ ID NO: 59 - protein, 37 aa, Artificial Sequence
  Designed polypeptide
KCNTATCATQRLANFLVHSSNNFGAILPSTKVGSNGY

SEQ ID NO: 60 - protein, 38 aa, Artificial Sequence
  Designed polypeptide
  misc_feature (38)..(38): X = Hyp (L-hydroxyproline)
KCNTATCTVQRLAHQIYQFTDKDKDNVAPPTKVGSNGX

SEQ ID NO: 61 - protein, 38 aa, Artificial Sequence; identical to SEQ ID NO: 14
  Designed polypeptide
  misc_feature (38)..(38): X = Hyp (L-hydroxyproline)

SEQ ID NO: 62 - protein, 38 aa, Artificial Sequence
  Designed polypeptide
  misc_feature (38)..(38): X = Hyp (L-hydroxyproline)
KCNTSTCTVQRLAHQIAQFTDKDKDNVAPPTNVGSNGX

SEQ ID NO: 63 - protein, 38 aa, Artificial Sequence
  Designed polypeptide
  misc_feature (38)..(38): X = Hyp (L-hydroxyproline)
KCNTATCTVQRLAEQIYQFTDKDKDNVAPPTAVGSNGX

SEQ ID NO: 64 - protein, 38 aa, Artificial Sequence
  Designed polypeptide
  misc_feature (38)..(38): X = Hyp (L-hydroxyproline)
KCNTATCTVQRLAHQIYQFTDADKDNVAPPTNVGSNGX

SEQ ID NO: 65 - protein, 37 aa, Artificial Sequence
  Designed polypeptide
  misc_feature (37)..(37): X = Hyp (L-hydroxyproline)
KCNTATCTVQRLAHQIYQFDKDKDNVAPPTNVGSNGX

SEQ ID NO: 66 - protein, 38 aa, Artificial Sequence
  Designed polypeptide
  misc_feature (38)..(38): X = Hyp (L-hydroxyproline)
KCNTATCTVQRLAEQIYQFTDKDKDNVSPPTNVGSNGX

SEQ ID NO: 67 - protein, 38 aa, Artificial Sequence; identical to SEQ ID NO: 17
  Designed polypeptide
  misc_feature (38)..(38): X = Hyp (L-hydroxyproline)

SEQ ID NO: 68 - protein, 38 aa, Artificial Sequence; identical to SEQ ID NO: 10
  Designed polypeptide
  misc_feature (38)..(38): X = Hyp (L-hydroxyproline)

SEQ ID NO: 69 - protein, 38 aa, Artificial Sequence
  Designed polypeptide
  misc_feature (38)..(38): X = Hyp (L-hydroxyproline)
KCNTATCTVQRLAHQIAQFTDKDKDNVAPPTNVGSNSX

SEQ ID NO: 70 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRTNVGPQGX

SEQ ID NO: 71 - protein, 38 aa, Artificial Sequence
  Designed polypeptide
  misc_feature (38)..(38): X = Hyp (L-hydroxyproline)
KCNTATCTVQRLAHQIAQYTDKDKDNVAPPTNVGSNGX

SEQ ID NO: 72 - protein, 38 aa, Artificial Sequence
  Designed polypeptide
  misc_feature (38)..(38): X = Hyp (L-hydroxyproline)
KCQTATCTVQRLAEQIYQFTDKDKDNVAPPTNVGSNGX

SEQ ID NO: 73 - protein, 38 aa, Artificial Sequence
  Designed polypeptide
  misc_feature (38)..(38): X = Hyp (L-hydroxyproline)
KCNTATCTVQRLAEQIYQFTDKDKDNVAPPTQVGSNGX

SEQ ID NO: 74 - protein, 38 aa, Artificial Sequence
  Designed polypeptide
  misc_feature (38)..(38): X = Hyp (L-hydroxyproline)
KCNTATCTVQRLAKQIYQFTDKDKDNVAPPTNVGSNGX

SEQ ID NO: 75 - protein, 38 aa, Artificial Sequence
  Designed polypeptide
  misc_feature (38)..(38): X = Hyp (L-hydroxyproline)
KCNTATCTVQRLAHQIYQFTDKDKDNVAPPTNVGSNGX

SEQ ID NO: 76 - protein, 35 aa, Artificial Sequence
  Designed polypeptide
  misc_feature (14)..(14): X = Aad (L-homoglutamic acid)
  misc_feature (22)..(22): X = Sar (Sarcosine)
  misc_feature (24)..(24): X = (NMe)I (N-methyl-L-isoleucine)
  misc_feature (35)..(35): X = Hyp (L-hydroxyproline)
RCGTATCATERLAXFLQRSSFXAXLSSTEVGSNTX

SEQ ID NO: 77 - protein, 38 aa, Artificial Sequence; identical to SEQ ID NO: 75
  Designed polypeptide
  misc_feature (38)..(38): X = Hyp (L-hydroxyproline)

SEQ ID NO: 78 - protein, 38 aa, Artificial Sequence
  Designed polypeptide
  misc_feature (38)..(38): X = Hyp (L-hydroxyproline)
KCNTATCTVQRLAEQIAQFTDKDKDNVAPPTNVGSNGX

SEQ ID NO: 79 - protein, 38 aa, Artificial Sequence
  Designed polypeptide
  misc_feature (38)..(38): X = Hyp (L-hydroxyproline)
KCNTATCTVQRLAHQIAQFSDKDKDNVAPPTNVGSNGX

SEQ ID NO: 80 - protein, 35 aa, Artificial Sequence
  Designed polypeptide
  misc_feature (22)..(22): X = Sar (Sarcosine)
  misc_feature (24)..(24): X = (NMe)I (N-methyl-L-isoleucine)
  misc_feature (35)..(35): X = Hyp (L-hydroxyproline)
RCQTATCATERLAHFLQRSSFXAXLSSTEVGSNTX

SEQ ID NO: 81 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRTNIGPNGX

SEQ ID NO: 82 - protein, 38 aa, Artificial Sequence
  Designed polypeptide
  misc_feature (38)..(38): X = Hyp (L-hydroxyproline)
KCNTATCTVQRLAHQIYQFTNKDKDNVAPPTNVGSNGX

SEQ ID NO: 83 - protein, 38 aa, Artificial Sequence; identical to SEQ ID NO: 23
  Designed polypeptide
  misc_feature (38)..(38): X = Hyp (L-hydroxyproline)

SEQ ID NO: 84 - protein, 38 aa, Artificial Sequence
  Designed polypeptide
GCRTGTCTVQRLAHQIYQFTDKDKDNVAPRTNVGPNGY

SEQ ID NO: 85 - protein, 38 aa, Artificial Sequence
  Designed polypeptide
  misc_feature (38)..(38): X = Hyp (L-hydroxyproline)
KCNTATCTVQRLAHQIVQFTDKDKDNVAPPTNVGSNGX

SEQ ID NO: 86 - protein, 38 aa, Artificial Sequence
  Designed polypeptide
  misc_feature (38)..(38): X = Hyp (L-hydroxyproline)
KCNTATCTVARLAHQINQFTDKDKDNVAPPTNVGSNGX

SEQ ID NO: 87 - protein, 38 aa, Artificial Sequence
  Designed polypeptide
  misc_feature (38)..(38): X = Hyp (L-hydroxyproline)
KCNTATCTVQRLAHQIAQFTDKNKDNVAPPTNVGSNGX

SEQ ID NO: 88 - protein, 38 aa, Artificial Sequence
  Designed polypeptide
  misc_feature (38)..(38): X = Hyp (L-hydroxyproline)
KCNTSTCTVQRLAHQIAQFTDKDKDPVAPPTNVGSNGX

SEQ ID NO: 89 - protein, 38 aa, Artificial Sequence
  Designed polypeptide
  misc_feature (38)..(38): X = Hyp (L-hydroxyproline)
KCNTATCTVQRLAEQIYQFKDKDKDNVAPPTNVGSNGX

SEQ ID NO: 90 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRTNVGPNGX

SEQ ID NO: 91 - protein, 38 aa, Artificial Sequence
  Designed polypeptide
  misc_feature (38)..(38): X = Hyp (L-hydroxyproline)
KCNTATCTVQRLAHQIYQFTDKNKDNVAPPTNVGSNGX

SEQ ID NO: 92 - protein, 38 aa, Artificial Sequence
  Designed polypeptide
  misc_feature (38)..(38): X = Hyp (L-hydroxyproline)
KCNTATCTVQRLAHQIAQFTDKDKDPVAPPTNVGSNGX

SEQ ID NO: 93 - protein, 37 aa, Artificial Sequence
  Designed polypeptide
KCNTATCATQRLANFLVHSSNNFGAIAPSTKVGSNGY

SEQ ID NO: 94 - protein, 38 aa, Artificial Sequence
  Designed polypeptide
  misc_feature (38)..(38): X = Hyp (L-hydroxyproline)
KCNTATCTVQRLAHQIAQFYDKDKDNVAPPTNVGSNGX

SEQ ID NO: 95 - protein, 38 aa, Artificial Sequence
  Designed polypeptide
  misc_feature (38)..(38): X = Hyp (L-hydroxyproline)
KCNTATCTVQRLAHQIAQFTNKDKDNVAPPTNVGSNGX

SEQ ID NO: 96 - protein, 38 aa, Artificial Sequence
  Designed polypeptide
  misc_feature (38)..(38): X = Hyp (L-hydroxyproline)
KCNTATCTVQRLAEQIYQFTDKDKDNVAPPTNVGSNTX

SEQ ID NO: 97 - protein, 38 aa, Artificial Sequence
  Designed polypeptide
  misc_feature (38)..(38): X = Hyp (L-hydroxyproline)
KCATATCTVQRLAEQIYQFTDKDKDNVAPPTNVGSNGX

SEQ ID NO: 98 - protein, 38 aa, Artificial Sequence
  Designed polypeptide
GCRFGTCTVQRLAHQIYQFTDKDKDNVAPRTNVSPNGY

SEQ ID NO: 99 - protein, 38 aa, Artificial Sequence; identical to SEQ ID NO: 20
  Designed polypeptide
  misc_feature (38)..(38): X = Hyp (L-hydroxyproline)

SEQ ID NO: 100 - protein, 38 aa, Artificial Sequence
  Designed polypeptide
  misc_feature (38)..(38): X = Hyp (L-hydroxyproline)
KCNTATCTVQRLAEQIYQFTDKDKDQVAPPTNVGSNGX

SEQ ID NO: 101 - protein, 38 aa, Artificial Sequence
  Designed polypeptide
  misc_feature (38)..(38): X = Hyp (L-hydroxyproline)
KCNAATCTVQRLAEQIYQFTDKDKDNVAPPTNVGSNGX

SEQ ID NO: 102 - protein, 38 aa, Artificial Sequence
  Designed polypeptide
KCNTATCTVQRLAHQIYQFTDKDKDNVAPRTEVGSNGY

SEQ ID NO: 103 - protein, 38 aa, Artificial Sequence
  Designed polypeptide
  misc_feature (38)..(38): X = Hyp (L-hydroxyproline)
KCNTATCTVVRLAHQIYQFTDKDKDNVAPPTNVGSNGX

SEQ ID NO: 104 - protein, 38 aa, Artificial Sequence
  Designed polypeptide
  misc_feature (38)..(38): X = Hyp (L-hydroxyproline)
KCNTATCTVQRLAHQIYQFYDKDKDNVAPPTNVGSNGX

SEQ ID NO: 105 - protein, 38 aa, Artificial Sequence
  Designed polypeptide
  misc_feature (38)..(38): X = Hyp (L-hydroxyproline)
KCNTATCTVQRLAHQIAQFADKDKDNVAPPTNVGSNGX

SEQ ID NO: 106 - protein, 38 aa, Artificial Sequence
  Designed polypeptide
  misc_feature (38)..(38): X = Hyp (L-hydroxyproline)
KCNTATCTVQRLAEQIYQFTAKDKDNVAPPTNVGSNGX

SEQ ID NO: 107 - protein, 38 aa, Artificial Sequence
  Designed polypeptide
  misc_feature (38)..(38): X = Hyp (L-hydroxyproline)
KCNTATCTVARLAHQIAQFTDKDKDNVAPPTNVGSNGX

SEQ ID NO: 108 - protein, 38 aa, Artificial Sequence
  Designed polypeptide
  misc_feature (38)..(38): X = Hyp (L-hydroxyproline)
KCNTATCTVQRLAEQIYQFTDKDKANVAPPTNVGSNGX

SEQ ID NO: 109 - protein, 38 aa, Artificial Sequence
  Designed polypeptide
GCRTGTCTVQRLAHQIYQFTDKDKDNVAPRTNVGPQGY

SEQ ID NO: 110 - protein, 38 aa, Artificial Sequence
  Designed polypeptide
  misc_feature (38)..(38): X = Hyp (L-hydroxyproline)
KCNTATCTVQRLAHQIEQFTDKDKDNVAPPTNVGSNGX

SEQ ID NO: 111 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRTKIGPQGX

SEQ ID NO: 112 - protein, 38 aa, Artificial Sequence
  Designed polypeptide
  misc_feature (38)..(38): X = Hyp (L-hydroxyproline)
KCNTATCTVDRLAHQIAQFTDKDKDNVAPPTNVGSNGX

SEQ ID NO: 113 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRTKISPNGX

SEQ ID NO: 114 - protein, 38 aa, Artificial Sequence
  Designed polypeptide
  misc_feature (38)..(38): X = Hyp (L-hydroxyproline)
KCNTATCTVQRLAHQIYQATDKDKDNVAPPTNVGSNGX

SEQ ID NO: 115 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRTKIGPNGX

SEQ ID NO: 116 - protein, 38 aa, Artificial Sequence
  Designed polypeptide
GCRFGTCTVQRLAHQIYQFTDKDKDNVAPRTNVSPQGY

SEQ ID NO: 117 - protein, 38 aa, Artificial Sequence
  Designed polypeptide
GCRFGTCTVQRLAHFLYQFTDKDKDNVAPRTKVGSNGY

SEQ ID NO: 118 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRTNIGPQGX

SEQ ID NO: 119 - protein, 38 aa, Artificial Sequence
  Designed polypeptide
  misc_feature (5)..(5): X = AIB (L-2-aminoisobutyric acid)
  misc_feature (38)..(38): X = Hyp (L-hydroxyproline)
KCNTXTCTVQRLAHQIYQFTDKDKDNVAPPTNVGSNGX

SEQ ID NO: 120 - protein, 38 aa, Artificial Sequence
  Designed polypeptide
  misc_feature (38)..(38): X = Hyp (L-hydroxyproline)
KCNTATCTVQRLAHQIAQFTDKDKDPVAPPTNVGSNSX

SEQ ID NO: 121 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRSNVGPQGX

SEQ ID NO: 122 - protein, 38 aa, Artificial Sequence
  Designed polypeptide
  misc_feature (38)..(38): X = Hyp (L-hydroxyproline)
KCNTATCTVQRLAEQIYQFTDKAKDNVAPPTNVGSNGX

SEQ ID NO: 123 - protein, 37 aa, Artificial Sequence
  Designed polypeptide
GCRFGTCTVQRLAHFLYQFTDKDGAIAPRTKIGSQGY

SEQ ID NO: 124 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRTKIGPNGX

SEQ ID NO: 125 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRTNVSPQGX

SEQ ID NO: 126 - protein, 38 aa, Artificial Sequence; identical to SEQ ID NO: 51
  Designed polypeptide
  misc_feature (38)..(38): X = Hyp (L-hydroxyproline)

SEQ ID NO: 127 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRSKIGPQGX

SEQ ID NO: 128 - protein, 37 aa, Artificial Sequence
  Designed polypeptide
KCNTATCATQRLANFLVHSSNNFGAIAPSTKIGPNGY

SEQ ID NO: 129 - protein, 38 aa, Artificial Sequence
  Designed polypeptide
GCRTGTCTVQRLAHQIYQFTDKDKDNVAPRSKVGPQGY

SEQ ID NO: 130 - protein, 38 aa, Artificial Sequence
  Designed polypeptide
  misc_feature (38)..(38): X = Hyp (L-hydroxyproline)
KCNTATCTVDRLAHQIAQFTDKDKDPVAPPTNVGSNGX

SEQ ID NO: 131 - protein, 38 aa, Artificial Sequence
  Designed polypeptide
GCRFGTCTVQRLAHQIYQFTDKDKDNVAPRSNVSPQGY

SEQ ID NO: 132 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRTKVSPQGX

SEQ ID NO: 133 - protein, 38 aa, Artificial Sequence
  Designed polypeptide
  misc_feature (38)..(38): X = Hyp (L-hydroxyproline)
KCNTATCTVARLAEQIYQFTDKDKDPVAPPTNVGSNGX

SEQ ID NO: 134 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRTKVSPNGX

SEQ ID NO: 135 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRSNVGPQGX

SEQ ID NO: 136 - protein, 38 aa, Artificial Sequence
  Designed polypeptide
  misc_feature (38)..(38): X = Hyp (L-hydroxyproline)
KCNTATCTVQRLAEQIKQFTDKDKDNVAPPTNVGSNGX

SEQ ID NO: 137 - protein, 38 aa, Artificial Sequence
  Designed polypeptide
  misc_feature (38)..(38): X = Hyp (L-hydroxyproline)
KCNTATCTVARLAHQINQFTDKDKDPVAPPTNVGSNGX

SEQ ID NO: 138 - protein, 38 aa, Artificial Sequence
  Designed polypeptide
GCRTGTCTVQRLAHQIYQFTDKDKDNVAPRSNVGPQGY

SEQ ID NO: 139 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRSKVGPNGX

SEQ ID NO: 140 - protein, 38 aa, Artificial Sequence
  Designed polypeptide
GCRFGTCTVQRLAHQIYQFTDKDKDNVAPRTNISPQGY

SEQ ID NO: 141 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRTNISPQGX

SEQ ID NO: 142 - protein, 38 aa, Artificial Sequence
  Designed polypeptide
GCRTGTCTVQRLAHQIYQFTDKDKDNVAPRTNIGPNGY

SEQ ID NO: 143 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRTKVGPQGX

SEQ ID NO: 144 - protein, 38 aa, Artificial Sequence
  Designed polypeptide
  misc_feature (38)..(38): X = Hyp (L-hydroxyproline)
KCNTATCTVQRLAEQIAQFTDKDKDPVAPPTNVGSNGX

SEQ ID NO: 145 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRSNVSPQGX

SEQ ID NO: 146 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRTKISPQGX

SEQ ID NO: 147 - protein, 38 aa, Artificial Sequence
  Designed polypeptide
  misc_feature (38)..(38): X = Hyp (L-hydroxyproline)
GCRIGTCTVQRLAHQIYQFTDKFKDNVAPRSKISPQTX

SEQ ID NO: 148 - protein, 37 aa, Artificial Sequence
  Designed polypeptide
KCNTATCATQRLANFLVHSSNNFGAILSSTKIGSNGY

SEQ ID NO: 149 - protein, 37 aa, Artificial Sequence
  Designed polypeptide
KCNTATCATQRLANFLVHSSNNFGAILPSTKVGPNGY

SEQ ID NO: 150 - protein, 38 aa, Artificial Sequence
  Designed polypeptide
  misc_feature (38)..(38): X = Hyp (L-hydroxyproline)
GCRWGTCTVQRLAHQIYQFTDKDKDNVAPRSKISPQTX

SEQ ID NO: 151 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRSNIGPNGX

SEQ ID NO: 152 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRSKVGPQGX

SEQ ID NO: 153 - protein, 38 aa, Artificial Sequence
  Designed polypeptide
  misc_feature (11)..(11): X = Cit (L-citrulline)
  misc_feature (38)..(38): X = Hyp (L-hydroxyproline)
KCNTATCTVQXLAEQIAQFTDKDKDNVAPPTNVGSNGX

SEQ ID NO: 154 - protein, 38 aa, Artificial Sequence
  Designed polypeptide
GCRFGTCTVQRLAHQIYQFTDKDKDNVAPRSKISPQGY

SEQ ID NO: 155 - protein, 38 aa, Artificial Sequence
  Designed polypeptide
GCRFGTCTVQRLAHQIYQFTDKDKDNVAPRTKVSPQGY

SEQ ID NO: 156 - protein, 38 aa, Artificial Sequence
  Designed polypeptide
GCRTGTCTVQRLAHQIYQFTDKDKDNVAPRTKVGPQGY

SEQ ID NO: 157 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRSKIGPNGX

SEQ ID NO: 158 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRSNISPQGX

SEQ ID NO: 159 - protein, 38 aa, Artificial Sequence
  Designed polypeptide
  misc_feature (38)..(38): X = Hyp (L-hydroxyproline)
KCNTATCTVIRLAHQIYQFTDKDKDNVAPPTNVGSNGX

SEQ ID NO: 160 - protein, 38 aa, Artificial Sequence
  Designed polypeptide
GCRTGTCTVQRLAHQIYQFTDKDKDNVAPRTNIGPQGY

SEQ ID NO: 161 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRTKIGPQGX

SEQ ID NO: 162 - protein, 37 aa, Artificial Sequence
  Designed polypeptide
KCNTATCATQRLANFLVHSSNNFGAILPSTKIGPNGY

SEQ ID NO: 163 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRTNVSPNGX

SEQ ID NO: 164 - protein, 38 aa, Artificial Sequence
  Designed polypeptide
GCRFGTCTVQRLAHQIYQFTDKDKDNVAPRTKISPQGY

SEQ ID NO: 165 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRSNIGPQGX

SEQ ID NO: 166 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRSNVSPNGX

SEQ ID NO: 167 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRSKVSPQGX

SEQ ID NO: 168 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRSKISPQTX

SEQ ID NO: 169 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRSKISPQGX

SEQ ID NO: 170 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRTNISPNGX

SEQ ID NO: 171 - protein, 38 aa, Artificial Sequence
  Designed polypeptide
  misc_feature (38)..(38): X = Hyp (L-hydroxyproline)
GCRWGTCTVQRLAHQIYQFTDKFKDNVAPRSKISPQTX

SEQ ID NO: 172 - protein, 38 aa, Artificial Sequence
  Designed polypeptide
GCRTGTCTVQRLAHQIYQFTDKDKDNVAPRSNVSPNGY

SEQ ID NO: 173 - protein, 38 aa, Artificial Sequence
  Designed polypeptide
  misc_feature (38)..(38): X = Hyp (L-hydroxyproline)
GCRMGTCTVQRLAHQIYQFTDKFKDNVAPRSKISPQTX

SEQ ID NO: 174 - protein, 38 aa, Artificial Sequence
  Designed polypeptide
GCRFGTCTVQRLAHQIYQFTDKDKDNVAPRSKVSPQGY

SEQ ID NO: 175 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRSKVSPNGX

SEQ ID NO: 176 - protein, 38 aa, Artificial Sequence
  Designed polypeptide
GCRTGTCTVQRLAHQIYQFTDKDKDNVAPRTNVSPQGY

SEQ ID NO: 177 - protein, 38 aa, Artificial Sequence
  Designed polypeptide
GCRTGTCTVQRLAHQIYQFTDKDKDNVAPRSNVGPNGY

SEQ ID NO: 178 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRTNVSPQGX

SEQ ID NO: 179 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRSKIGPQGX

SEQ ID NO: 180 - protein, 37 aa, Artificial Sequence
  Designed polypeptide
KCNTATCATQRLANFLVHSSNNFGAILPSTKIGSNTY

SEQ ID NO: 181 - protein, 37 aa, Artificial Sequence
  Designed polypeptide
  misc_feature (37)..(37): X = Hyp (L-hydroxyproline)
KCNTATCTVQRLAHQIYFTDKDKDNVAPPTNVGSNGX

SEQ ID NO: 182 - protein, 38 aa, Artificial Sequence
  Designed polypeptide
GCRFGTCTVQRLAHQIYQFTDKDKDNVAPRSNISPQGY

SEQ ID NO: 183 - protein, 38 aa, Artificial Sequence
  Designed polypeptide
GCRTGTCTVQRLAHQIYQFTDKDKDNVAPRSNVSPQGY

SEQ ID NO: 184 - protein, 38 aa, Artificial Sequence
  Designed polypeptide
GCRTGTCTVQRLAHQIYQFTDKFKDNVAPRSKISPQTW

SEQ ID NO: 185 - protein, 38 aa, Artificial Sequence
  Designed polypeptide
  misc_feature (38)..(38): X = Hyp (L-hydroxyproline)
GCRTGTCTVQRLAHQIYQFTDKFKDNVAPRSKISPQWX

SEQ ID NO: 186 - protein, 38 aa, Artificial Sequence
  Designed polypeptide
  misc_feature (38)..(38): X = Hyp (L-hydroxyproline)
GCRTGTCTVQRLAHQIYQFTDKFKDNVAPRSKISPQYX

SEQ ID NO: 187 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRSKVGPQGX

SEQ ID NO: 188 - protein, 38 aa, Artificial Sequence
  Designed polypeptide
GCRTGTCTVQRLAHQIYQFTDKDKDNVAPRTNISPNGY

SEQ ID NO: 189 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRTKISPQGX

SEQ ID NO: 190 - protein, 38 aa, Artificial Sequence
  Designed polypeptide
GCRTGTCTVQRLAHQIYQFTDKDKDNVAPRTKISPNGY

SEQ ID NO: 191 - protein, 38 aa, Artificial Sequence
  Designed polypeptide
GCRTGTCTVQRLAHQIYQFTDKDKDNVAPRSNIGPQGY

SEQ ID NO: 192 - protein, 38 aa, Artificial Sequence
  Designed polypeptide
  misc_feature (38)..(38): X = Hyp (L-hydroxyproline)
GCRMGTCTVQRLAHQIYQFTNKDKDNVAPRSKISPQTX

SEQ ID NO: 193 - protein, 38 aa, Artificial Sequence
  Designed polypeptide
  misc_feature (38)..(38): X = Hyp (L-hydroxyproline)
GCRTGTCTVQRLAHQIHQFTDKFKDNVAPRSKISPQTX

SEQ ID NO: 194 - protein, 38 aa, Artificial Sequence
  Designed polypeptide
  misc_feature (38)..(38): X = Hyp (L-hydroxyproline)
KCRMGTCTVQRLAHQIYQFTDKDKDNVAPRSKISPQTX

SEQ ID NO: 195 - protein, 37 aa, Artificial Sequence
  Designed polypeptide
  misc_feature (37)..(37): X = Hyp (L-hydroxyproline)
KCNTATCTVQRLAHQIYQTDKDKDNVAPPTNVGSNGX

SEQ ID NO: 196 - protein, 38 aa, Artificial Sequence
  Designed polypeptide
  misc_feature (38)..(38): X = Hyp (L-hydroxyproline)
GCRTGTCTVQRLAHQIAQFTDKFKDNVAPRSKISPQTX

SEQ ID NO: 197 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRTKISPNGX

SEQ ID NO: 198 - protein, 38 aa, Artificial Sequence
  Designed polypeptide
  misc_feature (38)..(38): X = Hyp (L-hydroxyproline)
GCRIGTCTVQRLAHQIYQFTNKDKDNVAPRSKISPQTX

SEQ ID NO: 199 - protein, 38 aa, Artificial Sequence
  Designed polypeptide
GCRTGTCTVQRLAHQIYQFTDKDKDNVAPRTNISPQGY

SEQ ID NO: 200 - protein, 38 aa, Artificial Sequence; identical to SEQ ID NO: 54
  Designed polypeptide

SEQ ID NO: 201 - protein, 38 aa, Artificial Sequence
  Designed polypeptide
  misc_feature (38)..(38): X = Hyp (L-hydroxyproline)
GCRTGTCTVQRLAHQIYQFTDKFKDNVAPRSKISPQHX

SEQ ID NO: 202 - protein, 38 aa, Artificial Sequence
  Designed polypeptide
  misc_feature (38)..(38): X = Hyp (L-hydroxyproline)
GCRWGTCTVQRLAHQIYQFTDKDKDNVAPSSKISPQTX

SEQ ID NO: 203 - protein, 38 aa, Artificial Sequence
  Designed polypeptide
  misc_feature (38)..(38): X = Hyp (L-hydroxyproline)
GCRTGTCTVQRLAHQIYQFTDKFKDNVAPRSKISPQPX

SEQ ID NO: 204 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRSKVSPNGX

SEQ ID NO: 205 - protein, 38 aa, Artificial Sequence
  Designed polypeptide
  misc_feature (38)..(38): X = Hyp (L-hydroxyproline)
KCRFGTCTVQRLAHQIYQFTDKDKDNVAPRSKISPQTX

SEQ ID NO: 206 - protein, 38 aa, Artificial Sequence
  Designed polypeptide
  misc_feature (38)..(38): X = Hyp (L-hydroxyproline)
GCRTGTCTVQRLAHQIYQFTDKFKDNVAPRSKISPQKX

SEQ ID NO: 207 - protein, 37 aa, Artificial Sequence
  Designed polypeptide
KCNTATCATQRLANFLVHSSNNFGAILPSTKIGSNGY

SEQ ID NO: 208 - protein, 38 aa, Artificial Sequence; identical to SEQ ID NO: 56
  Designed polypeptide

SEQ ID NO: 209 - protein, 38 aa, Artificial Sequence
  Designed polypeptide
GCRTGTCTVQRLAHQIYQFTDKDKDNVAPRTKVSPQGY

SEQ ID NO: 210 - protein, 38 aa, Artificial Sequence
  Designed polypeptide
  misc_feature (38)..(38): X = Hyp (L-hydroxyproline)
KCNTATCTVQRLAHQAYQFTDKDKDNVAPPTNVGSNGX

SEQ ID NO: 211 - protein, 38 aa, Artificial Sequence
  Designed polypeptide
  misc_feature (38)..(38): X = Hyp (L-hydroxyproline)
GCRTGTCTVQRLAHQIYQFTDKFKDNVAPRSKISPQFX

SEQ ID NO: 212 - protein, 38 aa, Artificial Sequence
  Designed polypeptide
  misc_feature (38)..(38): X = Hyp (L-hydroxyproline)
GCRFGTCTVQRLAHQIYQFTDKFKDNVAPRSKISPQTX

SEQ ID NO: 213 - protein, 38 aa, Artificial Sequence
  Designed polypeptide
  misc_feature (38)..(38): X = Hyp (L-hydroxyproline)
GCRTGTCTVQRLAHQIWQFTDKFKDNVAPRSKISPQTX

SEQ ID NO: 214 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRTNISPQGX

SEQ ID NO: 215 - protein, 38 aa, Artificial Sequence
  Designed polypeptide
GCRTGTCTVQRLAHQIYQFTDKFKDNVAPRSKISPQTM

SEQ ID NO: 216 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRTKVSPQGX

SEQ ID NO: 217 - protein, 38 aa, Artificial Sequence
  Designed polypeptide
  misc_feature (38)..(38): X = Hyp (L-hydroxyproline)
GCRFGTCTVQRLAHQLYQFTDKDKDNVAPRSKISPQTX

SEQ ID NO: 218 - protein, 38 aa, Artificial Sequence
  Designed polypeptide
  misc_feature (38)..(38): X = Hyp (L-hydroxyproline)
GCRTGTCTVQRLAHQIYQFTDKFKDNVAPRSKISPQAX

SEQ ID NO: 219 - protein, 38 aa, Artificial Sequence
  Designed polypeptide
  misc_feature (38)..(38): X = Hyp (L-hydroxyproline)
GCRTGTCTVQRLAHQIYQFTNKDKDNVAPRSKISPQHX

SEQ ID NO: 220 - protein, 38 aa, Artificial Sequence
  Designed polypeptide
  misc_feature (38)..(38): X = Hyp (L-hydroxyproline)
GCRTGTCTVQRLAHQIYQFTNKDKDNVAPRSKISPQWX

SEQ ID NO: 221 - protein, 38 aa, Artificial Sequence; identical to SEQ ID NO: 51
  Designed polypeptide
  misc_feature (38)..(38): X = Hyp (L-hydroxyproline)

SEQ ID NO: 222 - protein, 38 aa, Artificial Sequence
  Designed polypeptide
  misc_feature (38)..(38): X = Hyp (L-hydroxyproline)
GCRMGTCTVQRLAHQIYQFTDKDKDNVAPSSKISPQTX

SEQ ID NO: 223 - protein, 38 aa, Artificial Sequence; identical to SEQ ID NO: 117
  Designed polypeptide

SEQ ID NO: 224 - protein, 38 aa, Artificial Sequence
  Designed polypeptide
  misc_feature (38)..(38): X = Hyp (L-hydroxyproline)
GCRTGTCTVQRLAHQIYQFTDKDKDNVAPRSNVSPQGX

SEQ ID NO: 225 - protein, 38 aa, Artificial Sequence
  Designed polypeptide
  misc_feature (38)..(38): X = Hyp (L-hydroxyproline)
KCNTLTCTVQRLAHQIYQFTDKDKDNVAPPTNVGSNGX

SEQ ID NO: 226 - protein, 38 aa, Artificial Sequence
  Designed polypeptide
GCRTGTCTVQRLAHQIYQFTNKDKDNVAPRSKISPQTW

SEQ ID NO: 227 - protein, 38 aa, Artificial Sequence
  Designed polypeptide
  misc_feature (38)..(38): X = Hyp (L-hydroxyproline)
GCRTGTCTVQRLAHQIHQFTNKDKDNVAPRSKISPQTX

SEQ ID NO: 228 - protein, 38 aa, Artificial Sequence
  Designed polypeptide
  misc_feature (38)..(38): X = Hyp (L-hydroxyproline)
GCRTGTCTVQRLAHQIHQFTDKDKDNVAPRSKISPQTX

SEQ ID NO: 229 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPRSNIGPQGX

SEQ ID NO: 230 - protein, 38 aa, Artificial Sequence
  Designed polypeptide
GCRTGTCTVQRLAHQIYQFTDKFKDNVAPRSKISPQTY

SEQ ID NO: 231 - protein, 38 aa, Artificial Sequence
  Designed polypeptide
  misc_feature (38)..(38): X = Hyp (L-hydroxyproline)
KCNTYTCTVQRLAHQIYQFTDKDKDNVAPPTNVGSNGX

SEQ ID NO: 232 - protein, 38 aa, Artificial Sequence
  Designed polypeptide
  misc_feature (38)..(38): X = Hyp (L-hydroxyproline)
GCRTGTCTVQRLAHQIYQFTNKDKDNVAPRSKISPQQX

SEQ ID NO: 233 - protein, 38 aa, Artificial Sequence; identical to SEQ ID NO: 51
  Designed polypeptide
  misc_feature (38)..(38): X = Hyp (L-hydroxyproline)

SEQ ID NO: 234 - protein, 38 aa, Artificial Sequence
  Designed polypeptide
KCNTATCATQRLANFLVQFTDKDKDNVAPRSKISPQGY

SEQ ID NO: 235 - protein, 38 aa, Artificial Sequence
  Designed polypeptide
GCRTGTCTVQRLAHQIYQFTDKFKDNVAPRSKISPQTP

SEQ ID NO: 236 - protein, 38 aa, Artificial Sequence
  Designed polypeptide
  misc_feature (38)..(38): X = Hyp (L-hydroxyproline)
GCRTGTCTVQRLAHQIYQFTDKFKDNVAPRSKISPQMX

SEQ ID NO: 237 - protein, 38 aa, Artificial Sequence
  Designed polypeptide
GCRTGTCTVQRLAHQIYQFTNKDKDNVAPRSKISPQTM

SEQ ID NO: 238 - protein, 38 aa, Artificial Sequence
  Designed polypeptide
  misc_feature (38)..(38): X = Hyp (L-hydroxyproline)
GCRTGTCTVQRLAHQIYQFTNKDKDNVAPRSKISPQFX

SEQ ID NO: 239 - protein, 38 aa, Artificial Sequence
  Designed polypeptide
GCRTGTCTVQRLAHQIYQFTDKFKDNVAPRSKISPQTF

SEQ ID NO: 240 - protein, 38 aa, Artificial Sequence
  Designed polypeptide
  misc_feature (38)..(38): X = Hyp (L-hydroxyproline)
GCRFGTCTVQRLAHQIYQFTDKDKDNVAPSSKISPQTX

SEQ ID NO: 241 - protein, 38 aa, Artificial Sequence
  Designed polypeptide
GCRTGTCTVQRLAHQIYQFTDKFKDNVAPRSKISPQTA

SEQ ID NO: 242 - protein, 38 aa, Artificial Sequence
  Designed polypeptide
GCRTGTCTVQRLAHQIYQFTNKDKDNVAPRSKISPQTR

SEQ ID NO: 243 - protein, 38 aa, Artificial Sequence
  Designed polypeptide
  misc_feature (11)..(11): X = Cit (L-citrulline)
  misc_feature (38)..(38): X = Hyp (L-hydroxyproline)
KCNTATCTVQXLAAQIYQFTDKDKDNVAPPTNVGSNGX

SEQ ID NO: 244 - protein, 38 aa, Artificial Sequence
  Designed polypeptide
  misc_feature (11)..(11): X = Cit (L-citrulline)
  misc_feature (38)..(38): X = Hyp (L-hydroxyproline)
KCNTSTCTVAXLADQITQFSNKDKAQVSPPTEVGPNSX

SEQ ID NO: 245 - protein, 38 aa, Artificial Sequence
  Designed polypeptide
  misc_feature (11)..(11): X = Cit (L-citrulline)
  misc_feature (38)..(38): X = Hyp (L-hydroxyproline)
RCNASTCTVNXLADQITQFSNKDKAQVSPPTEVGPNSX

SEQ ID NO: 246 - protein, 38 aa, Artificial Sequence
  Designed polypeptide
  misc_feature (11)..(11): X = Cit (L-citrulline)
  misc_feature (38)..(38): X = Hyp (L-hydroxyproline)
KCNTATCTVQXLAHQIAQFTDKDKDNVAPPTNVGSNGX

SEQ ID NO: 247 - protein, 38 aa, Artificial Sequence
  Designed polypeptide
  misc_feature (11)..(11): X = Cit (L-citrulline)
  misc_feature (38)..(38): X = Hyp (L-hydroxyproline)
KCNTSTCTVAXLADQITQFSDKDKANVSPPTEVGPNSX

SEQ ID NO: 248 - protein, 38 aa, Artificial Sequence
  Designed polypeptide
  misc_feature (11)..(11): X = Cit (L-citrulline)
  misc_feature (38)..(38): X = Hyp (L-hydroxyproline)
KCNTATCTVQXLAHQIYQFTDKDKDNVAPRTKVGSNGX

SEQ ID NO: 249 - protein, 38 aa, Artificial Sequence
  Designed polypeptide
  misc_feature (11)..(11): X = Cit (L-citrulline)
  misc_feature (38)..(38): X = Hyp (L-hydroxyproline)
KCNTATCTVQXLAHQIYQFADKDKDNVAPPTNVGSNGX

SEQ ID NO: 250 - protein, 38 aa, Artificial Sequence
  Designed polypeptide
  misc_feature (11)..(11): X = Cit (L-citrulline)
  misc_feature (38)..(38): X = Hyp (L-hydroxyproline)
KCNTATCTVQXLAHQIYQFTDKDADNVAPPTNVGSNGX

SEQ ID NO: 251 - protein, 38 aa, Artificial Sequence
  Designed polypeptide
  misc_feature (11)..(11): X = Cit (L-citrulline)
  misc_feature (38)..(38): X = Hyp (L-hydroxyproline)
KCNTATCTVQXLAHQIYQFTDKDKDNAAPPTNVGSNGX

SEQ ID NO: 252 - protein, 38 aa, Artificial Sequence
  Designed polypeptide
  misc_feature (11)..(11): X = Cit (L-citrulline)
  misc_feature (38)..(38): X = Hyp (L-hydroxyproline)
KCNTATCTVQXLAHQIYQFTDKDKDNVAPATNVGSNGX

SEQ ID NO: 253 - protein, 37 aa, Artificial Sequence
  Designed polypeptide
  misc_feature (11)..(11): X = Cit (L-citrulline)
  misc_feature (37)..(37): X = Hyp (L-hydroxyproline)
KCNTATCTVQXLAHQIYQFTKDKDNVAPPTNVGSNGX

SEQ ID NO: 254 - protein, 37 aa, Artificial Sequence
  Designed polypeptide
  misc_feature (11)..(11): X = Cit (L-citrulline)
  misc_feature (37)..(37): X = Hyp (L-hydroxyproline)
KCNTATCTVQXLAHQIYQFTDKDKDNVPPTNVGSNGX

SEQ ID NO: 255 - protein, 37 aa, Artificial Sequence
  Designed polypeptide
  misc_feature (11)..(11): X = Cit (L-citrulline)
  misc_feature (37)..(37): X = Hyp (L-hydroxyproline)
KCNTATCTVQXLAHQIYQFTDKDKDNVAPTNVGSNGX

SEQ ID NO: 256 - protein, 38 aa, Artificial Sequence
  Designed polypeptide
  misc_feature (11)..(11): X = Cit (L-citrulline)
  misc_feature (38)..(38): X = Hyp (L-hydroxyproline)
RCQTSTCTVAXLAEQIAQYTDKDKDQVAPPTNVGSNSX

SEQ ID NO: 257 - protein, 38 aa, Artificial Sequence
  Designed polypeptide
  misc_feature (11)..(11): X = Cit (L-citrulline)
KCNTATCTVQXLAHQIYQFTDKDKDNVAPRTKVGSNGY

SEQ ID NO: 258 - protein, 38 aa, Artificial Sequence
  Designed polypeptide
  misc_feature (11)..(11): X = Cit (L-citrulline)
  misc_feature (38)..(38): X = Hyp (L-hydroxyproline)
KCNTATCTVQXLAHQIYQFTDKAKDNVAPPTNVGSNGX

SEQ ID NO: 259 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRFGTCTVQXLAHQIYQFTDKDKDNVAPRTKVGPQGX

SEQ ID NO: 260 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRFGTCTVQXLAHQIYQFTDKDKDNVAPRTNIGPQGX

SEQ ID NO: 261 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRTGTCTVQXLAHQIYQFTDKDKDNVAPRTNVGPQGX

SEQ ID NO: 262 - protein, 38 aa, Artificial Sequence
  Designed polypeptide
  misc_feature (11)..(11): X = Cit (L-citrulline)
  misc_feature (38)..(38): X = Hyp (L-hydroxyproline)
KCNTATCTVQXLAHQIYQFTDKDKDNVAAPTNVGSNGX

SEQ ID NO: 263 - protein, 38 aa, Artificial Sequence
  Designed polypeptide
  misc_feature (11)..(11): X = Cit (L-citrulline)
GCRFGTCTVQXLAHFLYQFTDKDKDNVAPRTNVGPQGY

SEQ ID NO: 264 - protein, 38 aa, Artificial Sequence
  Designed polypeptide
  misc_feature (11)..(11): X = Cit (L-citrulline)
GCRFGTCTVQXLAHFLYQFTDKDKDNVAPRTNVGSNTY

SEQ ID NO: 265 - protein, 37 aa, Artificial Sequence
  Designed polypeptide
  misc_feature (11)..(11): X = Cit (L-citrulline)
  misc_feature (37)..(37): X = Hyp (L-hydroxyproline)
KCNTATCTVQXLAHQIYQFDKDKDNVAPPTNVGSNGX

SEQ ID NO: 266 - protein, 38 aa, Artificial Sequence
  Designed polypeptide
  misc_feature (11)..(11): X = Cit (L-citrulline)
  misc_feature (38)..(38): X = Hyp (L-hydroxyproline)
KCNTATCTVQXLAEQIYQFTDKDKDNVSPPTNVGSNGX

SEQ ID NO: 267 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRFGTCTVQXLAHQIYQFTDKDKDNVAPRTNVGPQGX

SEQ ID NO: 268 - protein, 38 aa, Artificial Sequence
  Designed polypeptide
  misc_feature (11)..(11): X = Cit (L-citrulline)
  misc_feature (38)..(38): X = Hyp (L-hydroxyproline)
KCNTATCTVQXLAHQIYQFTDKDKDNVAPPTNVGSNGX

SEQ ID NO: 269 - protein, 38 aa, Artificial Sequence
  Designed polypeptide
  misc_feature (11)..(11): X = Cit (L-citrulline)
GCRTGTCTVQXLAHQIYQFTDKDKDNVAPRTNVGPNGY

SEQ ID NO: 270 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRFGTCTVQXLAHQIYQFTDKDKDNVAPRTNVGPNGX

SEQ ID NO: 271 - protein, 38 aa, Artificial Sequence
  Designed polypeptide
  misc_feature (11)..(11): X = Cit (L-citrulline)
GCRFGTCTVQXLAHQIYQFTDKDKDNVAPRTNVSPNGY

SEQ ID NO: 272 - protein, 38 aa, Artificial Sequence
  Designed polypeptide
  misc_feature (11)..(11): X = Cit (L-citrulline)
KCNTATCTVQXLAHQIYQFTDKDKDNVAPRTEVGSNGY

SEQ ID NO: 273 - protein, 38 aa, Artificial Sequence
  Designed polypeptide
  misc_feature (11)..(11): X = Cit (L-citrulline)
GCRTGTCTVQXLAHQIYQFTDKDKDNVAPRTNVGPQGY

SEQ ID NO: 274 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRFGTCTVQXLAHQIYQFTDKDKDNVAPRTKIGPQGX

SEQ ID NO: 275 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRFGTCTVQXLAHQIYQFTDKDKDNVAPRTKISPNGX

SEQ ID NO: 276 - protein, 38 aa, Artificial Sequence
  Designed polypeptide
  misc_feature (11)..(11): X = Cit (L-citrulline)
  misc_feature (38)..(38): X = Hyp (L-hydroxyproline)
KCNTATCTVQXLAHQIYQATDKDKDNVAPPTNVGSNGX

SEQ ID NO: 277 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRTGTCTVQXLAHQIYQFTDKDKDNVAPRTKIGPNGX

SEQ ID NO: 278 - protein, 38 aa, Artificial Sequence
  Designed polypeptide
  misc_feature (11)..(11): X = Cit (L-citrulline)
GCRFGTCTVQXLAHQIYQFTDKDKDNVAPRTNVSPQGY

SEQ ID NO: 279 - protein, 38 aa, Artificial Sequence
  Designed polypeptide
  misc_feature (11)..(11): X = Cit (L-citrulline)
GCRFGTCTVQXLAHFLYQFTDKDKDNVAPRTKVGSNGY

SEQ ID NO: 280 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRTGTCTVQXLAHQIYQFTDKDKDNVAPRTNIGPQGX

SEQ ID NO: 281 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRTGTCTVQXLAHQIYQFTDKDKDNVAPRSNVGPQGX

SEQ ID NO: 282 - protein, 37 aa, Artificial Sequence
  Designed polypeptide
  misc_feature (11)..(11): X = Cit (L-citrulline)
GCRFGTCTVQXLAHFLYQFTDKDGAIAPRTKIGSQGY

SEQ ID NO: 283 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRFGTCTVQXLAHQIYQFTDKDKDNVAPRTNVSPQGX

SEQ ID NO: 284 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRFGTCTVQXLAHQIYQFTDKDKDNVAPRSKIGPQGX

SEQ ID NO: 285 - protein, 38 aa, Artificial Sequence
  Designed polypeptide
  misc_feature (11)..(11): X = Cit (L-citrulline)
GCRTGTCTVQXLAHQIYQFTDKDKDNVAPRSKVGPQGY

SEQ ID NO: 286 - protein, 38 aa, Artificial Sequence
  Designed polypeptide
  misc_feature (11)..(11): X = Cit (L-citrulline)
GCRFGTCTVQXLAHQIYQFTDKDKDNVAPRSNVSPQGY

SEQ ID NO: 287 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRFGTCTVQXLAHQIYQFTDKDKDNVAPRTKVSPQGX

SEQ ID NO: 288 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRFGTCTVQXLAHQIYQFTDKDKDNVAPRTKVSPNGX

SEQ ID NO: 289 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRFGTCTVQXLAHQIYQFTDKDKDNVAPRSNVGPQGX

SEQ ID NO: 290 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRTGTCTVQXLAHQIYQFTDKDKDNVAPRSKVGPNGX

SEQ ID NO: 291 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRFGTCTVQXLAHQIYQFTDKDKDNVAPRTNISPQGX

SEQ ID NO: 292 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRTGTCTVQXLAHQIYQFTDKDKDNVAPRTKVGPQGX

SEQ ID NO: 293 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRFGTCTVQXLAHQIYQFTDKDKDNVAPRSNVSPQGX

SEQ ID NO: 294 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRFGTCTVQXLAHQIYQFTDKDKDNVAPRTKISPQGX

SEQ ID NO: 295 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRIGTCTVQXLAHQIYQFTDKFKDNVAPRSKISPQTX

SEQ ID NO: 296 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRWGTCTVQXLAHQIYQFTDKDKDNVAPRSKISPQTX

SEQ ID NO: 297 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRTGTCTVQXLAHQIYQFTDKDKDNVAPRSNIGPNGX

SEQ ID NO: 298 - protein, 38 aa, Artificial Sequence
  Designed polypeptide
  misc_feature (11)..(11): X = Cit (L-citrulline)
GCRTGTCTVQXLAHQIYQFTDKDKDNVAPRTKVGPQGY

SEQ ID NO: 299 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRTGTCTVQXLAHQIYQFTDKDKDNVAPRSKIGPNGX

SEQ ID NO: 300 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRFGTCTVQXLAHQIYQFTDKDKDNVAPRSNISPQGX

SEQ ID NO: 301 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRTGTCTVQXLAHQIYQFTDKDKDNVAPRTKIGPQGX

SEQ ID NO: 302 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRTGTCTVQXLAHQIYQFTDKDKDNVAPRTNVSPNGX

SEQ ID NO: 303 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRTGTCTVQXLAHQIYQFTDKDKDNVAPRSNIGPQGX

SEQ ID NO: 304 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRTGTCTVQXLAHQIYQFTDKDKDNVAPRSNVSPNGX

SEQ ID NO: 305 - protein, 38 aa, Artificial Sequence
  Designed polypeptide
  misc_feature (11)..(11): X = Cit (L-citrulline)
  misc_feature (38)..(38): X = Hyp (L-hydroxyproline)
GCRFGTCTVQXLAHQIYQFTDKDKDNVAPRSKVSPQGX

SEQ ID NO: 306 - protein, 38 aa, Artificial Sequence
  Designed polypeptide
  misc_feature (38)..(38): X = Hyp (L-hydroxyproline)
KCNTSTCTVAWLADQITQFSNKDKAQVSPPTEVGPNSX

SEQ ID NO: 307 - protein, 38 aa, Artificial Sequence
  Designed polypeptide
  misc_feature (38)..(38): X = Hyp (L-hydroxyproline)
RCNASTCTVNWLADQITQFSNKDKAQVSPPTEVGPNSX

SEQ ID NO: 308 - protein, 38 aa, Artificial Sequence
  Designed polypeptide
  misc_feature (38)..(38): X = Hyp (L-hydroxyproline)
KCNTATCTVQWLAHAIYQFTDKDKDNVAPPTNVGSNGX

SEQ ID NO: 309 - protein, 38 aa, Artificial Sequence
  Designed polypeptide
  misc_feature (38)..(38): X = Hyp (L-hydroxyproline)
KCNTATCTVQWLAHQIAQFTDKDKDNVAPPTNVGSNGX

SEQ ID NO: 310 - protein, 38 aa, Artificial Sequence
  Designed polypeptide
  misc_feature (38)..(38): X = Hyp (L-hydroxyproline)
KCNTSTCTVAWLADQITQFSDKDKANVSPPTEVGPNSX

SEQ ID NO: 311 - protein, 38 aa, Artificial Sequence
  Designed polypeptide
GCRFGTCTVQWLANFLYQFTDKDKDNVAPRTNVGPQGY

SEQ ID NO: 312 - protein, 38 aa, Artificial Sequence
  Designed polypeptide
  misc_feature (38)..(38): X = Hyp (L-hydroxyproline)
KCNTATCTVQWLAHQIYQFTDKDKDNVAPRTKVGSNGX

SEQ ID NO: 313 - protein, 38 aa, Artificial Sequence
  Designed polypeptide
  misc_feature (38)..(38): X = Hyp (L-hydroxyproline)
KCNTATCTVQWLAHQIYQFADKDKDNVAPPTNVGSNGX

SEQ ID NO: 314 - protein, 38 aa, Artificial Sequence
  Designed polypeptide
  misc_feature (38)..(38): X = Hyp (L-hydroxyproline)
KCNTATCTVQWLAHQIYQFTDKDKANVAPPTNVGSNGX

SEQ ID NO: 315 - protein, 38 aa, Artificial Sequence
  Designed polypeptide
  misc_feature (38)..(38): X = Hyp (L-hydroxyproline)
KCNTATCTVQWLAHQIYQFTDKDKDNAAPPTNVGSNGX

SEQ ID NO: 316 - protein, 38 aa, Artificial Sequence
  Designed polypeptide
  misc_feature (38)..(38): X = Hyp (L-hydroxyproline)
KCNTATCTVQWLAHQIYQFTDKDKDNVAPATNVGSNGX

SEQ ID NO: 317 - protein, 37 aa, Artificial Sequence
  Designed polypeptide
  misc_feature (37)..(37): X = Hyp (L-hydroxyproline)
KCNTATCTVQWLAHQIYQFTKDKDNVAPPTNVGSNGX

SEQ ID NO: 318 - protein, 37 aa, Artificial Sequence
  Designed polypeptide
  misc_feature (37)..(37): X = Hyp (L-hydroxyproline)
KCNTATCTVQWLAHQIYQFTDKDKDNVPPTNVGSNGX

SEQ ID NO: 319 - protein, 37 aa, Artificial Sequence
  Designed polypeptide
  misc_feature (37)..(37): X = Hyp (L-hydroxyproline)
KCNTATCTVQWLAHQIYQFTDKDKDNVAPTNVGSNGX

SEQ ID NO: 320 - protein, 38 aa, Artificial Sequence
  Designed polypeptide
  misc_feature (38)..(38): X = Hyp (L-hydroxyproline)
KCNTATCTVQWLAHQIYQFTDKAKDNVAPPTNVGSNGX

SEQ ID NO: 321 - protein, 38 aa, Artificial Sequence
  Designed polypeptide
  misc_feature (38)..(38): X = Hyp (L-hydroxyproline)
KCNTATCTVQWLAEQIYQFTDKDKDNVAPPYNEGSNGX

SEQ ID NO: 322 - protein, 38 aa, Artificial Sequence
  Designed polypeptide
  misc_feature (38)..(38): X = Hyp (L-hydroxyproline)
GCRFGTCTVQWLAHQIYQFTDKDKDNVAPRTKVGPQGX

SEQ ID NO: 323 - protein, 38 aa, Artificial Sequence
  Designed polypeptide
  misc_feature (38)..(38): X = Hyp (L-hydroxyproline)
GCRTGTCTVQWLAHQIYQFTDKDKDNVAPRTNVGPQGX

SEQ ID NO: 324 - protein, 38 aa, Artificial Sequence
  Designed polypeptide
  misc_feature (38)..(38): X = Hyp (L-hydroxyproline)
KCNTATCTVQWLAHQIYQFTDKDKDAVAPPTNVGSNGX

SEQ ID NO: 325 - protein, 38 aa, Artificial Sequence
  Designed polypeptide
  misc_feature (38)..(38): X = Hyp (L-hydroxyproline)
KCNTATCTVQWLAHQIYQFTDKDKDNVAAPTNVGSNGX

SEQ ID NO: 326 - protein, 38 aa, Artificial Sequence
  Designed polypeptide
GCRFGTCTVQWLAHFLYQFTDKDKDNVAPRTNVGPQGY

SEQ ID NO: 327 - protein, 38 aa, Artificial Sequence
  Designed polypeptide
GCRFGTCTVQWLAHFLYQFTDKDKDNVAPRTNVGSNTY

SEQ ID NO: 328 - protein, 38 aa, Artificial Sequence
  Designed polypeptide
  misc_feature (38)..(38): X = Hyp (L-hydroxyproline)
KCNTATCTVQWLAHQIYQFTDADKDNVAPPTNVGSNGX

SEQ ID NO: 329 - protein, 37 aa, Artificial Sequence
  Designed polypeptide
  misc_feature (37)..(37): X = Hyp (L-hydroxyproline)
KCNTATCTVQWLAHQIYQFDKDKDNVAPPTNVGSNGX

SEQ ID NO: 330 - protein, 38 aa, Artificial Sequence
  Designed polypeptide
  misc_feature (38)..(38): X = Hyp (L-hydroxyproline)
KCNTATCTVQWLAHQIYQFTDKDKDNVAPPTNVGSNGX

SEQ ID NO: 331 - protein, 38 aa, Artificial Sequence
  Designed polypeptide
  misc_feature (38)..(38): X = Hyp (L-hydroxyproline)
GCRFGTCTVQWLAHQIYQFTDKDKDNVAPRTNIGPNGX

SEQ ID NO: 332 - protein, 38 aa, Artificial Sequence
  Designed polypeptide
GCRTGTCTVQWLAHQIYQFTDKDKDNVAPRTNVGPNGY

SEQ ID NO: 333 - protein, 38 aa, Artificial Sequence
  Designed polypeptide
  misc_feature (38)..(38): X = Hyp (L-hydroxyproline)
KCNAATCTVQWLAEQIYQFTDKDKDNVAPPTNVGSNGX

SEQ ID NO: 334 - protein, 38 aa, Artificial Sequence
  Designed polypeptide
KCNTATCTVQWLAHQIYQFTDKDKDNVAPRTEVGSNGY

SEQ ID NO: 335 - protein, 38 aa, Artificial Sequence
  Designed polypeptide
GCRTGTCTVQWLAHQIYQFTDKDKDNVAPRTNVGPQGY

SEQ ID NO: 336 - protein, 38 aa, Artificial Sequence
  Designed polypeptide
  misc_feature (38)..(38): X = Hyp (L-hydroxyproline)
GCRFGTCTVQWLAHQIYQFTDKDKDNVAPRTKIGPQGX

SEQ ID NO: 337 - protein, 38 aa, Artificial Sequence
  Designed polypeptide
  misc_feature (38)..(38): X = Hyp (L-hydroxyproline)
GCRFGTCTVQWLAHQIYQFTDKDKDNVAPRTKISPNGX

SEQ ID NO: 338 - protein, 38 aa, Artificial Sequence
  Designed polypeptide
  misc_feature (38)..(38): X = Hyp (L-hydroxyproline)
KCNTATCTVQWLAHQIYQATDKDKDNVAPPTNVGSNGX

SEQ ID NO: 339 - protein, 38 aa, Artificial Sequence
  Designed polypeptide
  misc_feature (38)..(38): X = Hyp (L-hydroxyproline)
GCRTGTCTVQWLAHQIYQFTDKDKDNVAPRTKIGPNGX

SEQ ID NO: 340 - protein, 38 aa, Artificial Sequence
  Designed polypeptide
GCRFGTCTVQWLAHQIYQFTDKDKDNVAPRTNVSPQGY

SEQ ID NO: 341 - protein, 38 aa, Artificial Sequence
  Designed polypeptide
  misc_feature (38)..(38): X = Hyp (L-hydroxyproline)
GCRTGTCTVQWLAHQIYQFTDKDKDNVAPRTNIGPQGX

SEQ ID NO: 342 - protein, 38 aa, Artificial Sequence
  Designed polypeptide
  misc_feature (38)..(38): X = Hyp (L-hydroxyproline)
GCRTGTCTVQWLAHQIYQFTDKDKDNVAPRSNVGPQGX

SEQ ID NO: 343 - protein, 38 aa, Artificial Sequence
  Designed polypeptide
  misc_feature (38)..(38): X = Hyp (L-hydroxyproline)
GCRFGTCTVQWLAHQIYQFTDKDKDNVAPRTKIGPNGX

SEQ ID NO: 344 - protein, 38 aa, Artificial Sequence
  Designed polypeptide
GCRTGTCTVQWLAHQIYQFTDKDKDNVAPRSKVGPQGY

SEQ ID NO: 345 - protein, 38 aa, Artificial Sequence
  Designed polypeptide
GCRFGTCTVQWLAHQIYQFTDKDKDNVAPRSNVSPQGY

SEQ ID NO: 346 - protein, 38 aa, Artificial Sequence
  Designed polypeptide
GCRTGTCTVQWLAHQIYQFTDKDKDNVAPRSNVGPQGY

SEQ ID NO: 347 - protein, 38 aa, Artificial Sequence
  Designed polypeptide
  misc_feature (38)..(38): X = Hyp (L-hydroxyproline)
GCRTGTCTVQWLAHQIYQFTDKDKDNVAPRSKVGPNGX

SEQ ID NO: 348 - protein, 38 aa, Artificial Sequence
  Designed polypeptide
GCRTGTCTVQWLAHQIYQFTDKDKDNVAPRTNIGPNGY

SEQ ID NO: 349 - protein, 38 aa, Artificial Sequence
  Designed polypeptide
  misc_feature (38)..(38): X = Hyp (L-hydroxyproline)
GCRTGTCTVQWLAHQIYQFTDKDKDNVAPRTKVGPQGX

SEQ ID NO: 350 - protein, 38 aa, Artificial Sequence
  Designed polypeptide
  misc_feature (38)..(38): X = Hyp (L-hydroxyproline)
GCRFGTCTVQWLAHQIYQFTDKDKDNVAPRTKISPQGX

SEQ ID NO: 351 - protein, 38 aa, Artificial Sequence
  Designed polypeptide
  misc_feature (38)..(38): X = Hyp (L-hydroxyproline)
GCRIGTCTVQWLAHQIYQFTDKFKDNVAPRSKISPQTX

SEQ ID NO: 352 - protein, 38 aa, Artificial Sequence
  Designed polypeptide
  misc_feature (38)..(38): X = Hyp (L-hydroxyproline)
GCRTGTCTVQWLAHQIYQFTDKDKDNVAPRSNIGPNGX

SEQ ID NO: 353 - protein, 38 aa, Artificial Sequence
  Designed polypeptide
  misc_feature (38)..(38): X = Hyp (L-hydroxyproline)
GCRFGTCTVQWLAHQIYQFTDKDKDNVAPRSKVGPQGX

SEQ ID NO: 354 - protein, 38 aa, Artificial Sequence
  Designed polypeptide
GCRTGTCTVQWLAHQIYQFTDKDKDNVAPRTKVGPQGY

SEQ ID NO: 355 - protein, 38 aa, Artificial Sequence
  Designed polypeptide
  misc_feature (38)..(38): X = Hyp (L-hydroxyproline)
GCRTGTCTVQWLAHQIYQFTDKDKDNVAPRSKIGPNGX

SEQ ID NO: 356 - protein, 38 aa, Artificial Sequence
  Designed polypeptide
GCRTGTCTVQWLAHQIYQFTDKDKDNVAPRTNIGPQGY

SEQ ID NO: 357 - protein, 38 aa, Artificial Sequence
  Designed polypeptide
  misc_feature (38)..(38): X = Hyp (L-hydroxyproline)
GCRTGTCTVQWLAHQIYQFTDKDKDNVAPRTKIGPQGX

SEQ ID NO: 358 - protein, 38 aa, Artificial Sequence
  Designed polypeptide
  misc_feature (38)..(38): X = Hyp (L-hydroxyproline)
GCRTGTCTVQWLAHQIYQFTDKDKDNVAPRTNVSPNGX

SEQ ID NO: 359 - protein, 38 aa, Artificial Sequence
  Designed polypeptide
  misc_feature (38)..(38): X = Hyp (L-hydroxyproline)
GCRTGTCTVQWLAHQIYQFTDKDKDNVAPRSNIGPQGX

SEQ ID NO: 360 - protein, 38 aa, Artificial Sequence
  Designed polypeptide
  misc_feature (38)..(38): X = Hyp (L-hydroxyproline)
GCRTGTCTVQWLAHQIYQFTDKDKDNVAPRSNVSPNGX

SEQ ID NO: 361 - protein, 38 aa, Artificial Sequence
  Designed polypeptide
KCNFATCTVQRLAHQIYQFTDKDKDNVAPRTKVGSNGY

SEQ ID NO: 362 - protein, 38 aa, Artificial Sequence
  Designed polypeptide
GCRFGTCTVQRLAHQIYQFTDKDKDNVAPRTNVGPQGY

SEQ ID NO: 363 - protein, 38 aa, Artificial Sequence
  Designed polypeptide
KCNFATCATQRLANFLVQFTDKDKDNVAPRSKIGPQGY

SEQ ID NO: 364 - protein, 38 aa, Artificial Sequence
  Designed polypeptide
GCRFGTCTVQRLAHFLYQFTDKDKDNVAPRTNIGPQGY

SEQ ID NO: 365 - protein, 38 aa, Artificial Sequence
  Designed polypeptide
GCRFGTCTVQRLAHFLYQFTDKDKDNVAPRTKIGPQGY

SEQ ID NO: 366 - protein, 38 aa, Artificial Sequence
  Designed polypeptide
GCRFGTCTVQRLAHQIYQFTDKDKDNVAPRTNISPNGY

SEQ ID NO: 367 - protein, 38 aa, Artificial Sequence
  Designed polypeptide
GCRFGTCTVQRLAHQIYQFTDKDKDNVAPRTKIGPNGY

SEQ ID NO: 368 - protein, 38 aa, Artificial Sequence
  Designed polypeptide
GCRFGTCTVQRLAHQIYQFTDKDKDNVAPRTKVGPQGY

SEQ ID NO: 369 - protein, 38 aa, Artificial Sequence
  Designed polypeptide
GCRFGTCTVQRLAHFLYQFTDKDKDNVAPRTKVGPQGY

SEQ ID NO: 370 - protein, 38 aa, Artificial Sequence
  Designed polypeptide
KCNFATCTVQRLAHQIYQFTDKDKDNVAPRTEVGSNGY

SEQ ID NO: 371 - protein, 38 aa, Artificial Sequence
  Designed polypeptide
GCRFGTCTVQRLAHQIYQFTDKDKDNVAPRTKISPNGY

SEQ ID NO: 372 - protein, 38 aa, Artificial Sequence
  Designed polypeptide
GCRFGTCTVQRLANQIYQFTDKDKDNVAPRTNVGPQGY

SEQ ID NO: 373 - protein, 38 aa, Artificial Sequence
  Designed polypeptide
GCRFGTCTVQRLAHQIYQFTDKDKDNVAPRSNVGPNGY

SEQ ID NO: 374 - protein, 38 aa, Artificial Sequence
  Designed polypeptide
GCRFGTCTVQRLAHQIYQFTDKDKDNVAPRSNVGPQGY

SEQ ID NO: 375 - protein, 38 aa, Artificial Sequence
  Designed polypeptide
GCRFGTCTVQRLAHQIYQFTDKDKDNVAPRSKVGPQGY

SEQ ID NO: 376 - protein, 38 aa, Artificial Sequence
  Designed polypeptide
GCRFGTCTVQRLAHFLYQFTDKDKDNVAPRTKIGSNGY

SEQ ID NO: 377 - protein, 38 aa, Artificial Sequence
  Designed polypeptide
GCRFATCTVQRLAHFLYQFTDKDKDNVAPRTKVGPQGY

SEQ ID NO: 378 - protein, 38 aa, Artificial Sequence; identical to SEQ ID NO: 361
  Designed polypeptide

SEQ ID NO: 379 - protein, 38 aa, Artificial Sequence
  Designed polypeptide
GCRFGTCTVQRLAHQIYQFTDKDKDNVAPRTNIGPQGY

SEQ ID NO: 380 - protein, 38 aa, Artificial Sequence
  Designed polypeptide
GCRFATCTVQRLAHQIYQFTDKDKDNVAPRTKVGPQGY

SEQ ID NO: 381 - protein, 38 aa, Artificial Sequence
  Designed polypeptide
GCRFGTCTVQRLAHQIYQFTDKDKDNVAPRTNIGPNGY

SEQ ID NO: 382 - protein, 38 aa, Artificial Sequence
  Designed polypeptide
GCRFGTCTVQRLAHQIYQFTDKDKDNVAPRSNIGPQGY

SEQ ID NO: 383 - protein, 38 aa, Artificial Sequence
  Designed polypeptide
GCRFGTCTVQRLAHQIYQFTDKDKDNVAPRSNISPNGY

SEQ ID NO: 384 - protein, 38 aa, Artificial Sequence
  Designed polypeptide
GCRFGTCTVQRLAHQIYQFTDKDKDNVAPRSKVSPNGY

SEQ ID NO: 385 - protein, 38 aa, Artificial Sequence
  Designed polypeptide
GCRFGTCTVQRLAHQIYQFTDKDKDNVAPRTKVSPNGY

SEQ ID NO: 386 - protein, 38 aa, Artificial Sequence; identical to SEQ ID NO: 16
  Designed polypeptide

SEQ ID NO: 387 - protein, 38 aa, Artificial Sequence
  Designed polypeptide
GCRFGTCTVQRLAHQIYQFTDKDKDNVAPRSKIGPQGY

SEQ ID NO: 388 - protein, 38 aa, Artificial Sequence; identical to SEQ ID NO: 364
  Designed polypeptide

SEQ ID NO: 389 - protein, 38 aa, Artificial Sequence; identical to SEQ ID NO: 363
  Designed polypeptide

SEQ ID NO: 390 - protein, 38 aa, Artificial Sequence; identical to SEQ ID NO: 365
  Designed polypeptide

SEQ ID NO: 391 - protein, 38 aa, Artificial Sequence
  Designed polypeptide
GCRFGTCTVQRLAHQIYQFTDKDKDNVAPRTKIGPQGY

SEQ ID NO: 392 - protein, 38 aa, Artificial Sequence; identical to SEQ ID NO: 369
  Designed polypeptide